(19) United States
(12) Patent Application Publication
(10) Pub. No.: US 2008/0175822 A1
(43) Pub. Date: Jul. 24, 2008

(54) Non-human primate embryonic stem and germ cells: methods of use and methods of making same

(76) Inventors: Gerald P. Schatten, Pittsburgh, PA (US); Calvin R. Simerly, Cranberry, PA (US); Christopher S. Navara, Cranberry, PA (US)

Correspondence Address: DON J. PELTO;Sheppard, Mullin, Richter & Hampton LLP, 1300 I STREET, NW, 11TH FLOOR EAST, WASHINGTON, DC, 20005, US

(21) Appl. No.: 11/655,146

(22) Filed: Jan. 19, 2007

(51) Int. Cl. A61K 35/12; A01N 1/02; C12N 5/06; C12Q 1/02

(52) U.S. Cl. 424/93.7; 435/1.1; 435/29; 435/325

(57) ABSTRACT

The present invention provides a non-human primate (nhp) pluripotential embryonic stem (ES) cell, which can be used in several ways as described herein, including to generate chimeric primate embryos. The invention further provides methods to determine the differentiation status of an embryonic cell by comparing its transcriptional patterns with those of ES cells at particular stages of differentiation.

The invention further provides a non-human primate embryonic germ (EG) cell which can also be used in several ways, including administering the differentiated EG cell line to a patient to treat a number of diseases.

Also provided are methods of generating nhp ES cell+primate embryo chimeras, and methods of deriving EG cells.

### FIELD OF THE INVENTION

The present invention relates to the study and development of non-human primate stem cells.

### BACKGROUND OF THE INVENTION

Responsible realization of human embryonic stem cells' (hES cells) biomedical promises is an extremely important research goal. hES cells promise previously unimagined therapies for devastating disorders, and enable unique scientific opportunities to responsibly discover the fundamental mechanisms of healthy human development.

The eventual clinical progression of embryonic stem cell (ESCs) derived tissues for therapeutic applications will require extensive studies of safety and efficacy in both small and large animal system. Because of the extensive evolutionary distance between rodents and humans, and already evident differences between mouse and primate ESCs, I there are limits to the use of mouse ESCs to demonstrate safety and efficacy of stem cell-based transplants. Therefore, the present experiments rely heavily on non-human primate as the most relevant model for assessing the therapeutic potential of pluripotent stem cells. While a number of such species are germane options, several considerations point to Rhesus monkeys: their high evolutionary relatedness to humans; the scope and availability of existing breeding stocks; and the progression of the Macaque genome project, with imminent production of a Rhesus Genechip.

The goals of the research described herein are: (i) To understand the fundamental biology of pluripotent stem cells during development and differentiation by investigating the NIH-approved hES cell lines and as additional models, deriving and studying nonhuman primate ES cells (nonhuman primate embryonic and germ cell lines) and; (ii) To determine the transplantation potentials of ES cells by non-invasively imaging autografts, allografts, and xenografts into primates and mice, with special attention devoted to focus on the safety of differentiated stem cell transplantation of nhp-ESCs derived from NT embryos, as well as immune tolerance.

### SUMMARY OF THE INVENTION

The present invention provides a composition comprised of a chimeric primate embryo derived from non-human ES cells plus a fertilized primate embryo.

The present invention further provides a method of generating a chimeric primate embryo, which comprises reaggregating nhp ES cells with biopsied fertilized primate embryos.

The present invention provides a method of determining the differentiation status of an embryonic cell. In a specific embodiment, this method comprises the steps of determining the cell transcriptional pattern of the embyronic cell; comparing this embryonic cell transcriptional pattern to various prototype transcriptional patterns, where each of these prototype transcriptional patterns are derived from an embryonic cell of a specific embryonic cell differentiation status; determining which of these prototype transcriptional patterns most closely resembles the embryonic cell transcriptional pattern; and finally assigning the specific embryonic cell differentiation status corresponding to the prototype transcriptional pattern most closely matching the embryonic cell transcriptional pattern, to the embryonic cell.

In another specific embodiment of the present invention, the embryonic cell transcriptional pattern is determined by hybridizing labeled RNA, isolated from single colonies of cells, to microarray chips. In yet another specific embodiment of the present invention, these microarray chips display genomic DNA fragments originating from a species selected from the group consisting of mouse, human and Rhesus monkey. In another embodiment, these microarray chips are selected from the group consisting of the Affymetrix® hg-u133+2 chip, Affymetrix® GeneChip® Rhesus Macaque Genome Array and Affymetrix® mg-u74Av2 chip.

In another specific embodiment, the specific embryonic cell differentiation status determined by the method of the present invention can be the inner cell mast, the epiblast, the mesoderm, endoderm, ectoderm, lateral plate mesoderm, gut and neuroectoderm, extraembryonic mesoderm, amniotic ectoderm and visceral endoderm.

The present invention provides the composition of an isolated nhp pluripotential EG cell, methods for deriving this cell and methods for the use of this cell in the treatment and/or prevention of various diseases.

In a specific embodiment of the present invention, a method is provided for deriving nhp EG cells, wherein non-human primates are mated to establish pregnancy; the pregnancy is terminated at between 28 and 45 days and an nhp fetus obtained; gonads are isolated from this fetus and placed on plates of feeder cells; the plates are cultured, then fixed and stained for TNAP, a marker for PGCs; the PGCs are detected by TNAP staining; the PGCs placed into culture, supplemented with LIF, bFGF and forskolin; the culture is fed daily; EG cells are isolated by picking, then plated singly; and the EG colonies resulting are then tested for specific EG-expressed markers.

The present invention also provides for the composition that is a differentiated cell derived from an nhp EG cell. Further, the present invention provides for the composition that is the embryoid body derived from an nhp EG cell.

The present invention also provides for a method of administering a composition comprised of the differentiated cell derived from an nhp EG cell, in order to treat, prevent and/or alleviate the occurrence or negative effects of disease. In a specific embodiment of the present invention, this disease may be Alzheimer's, Parkinson's, muscular dystrophy, diabetes, stroke, and/or cardiovascular disease such as congestive heart failure, ischemic heart disease, cardiomyopathy, hypertension, coronary artery disease, high blood pressure, arrhythmia, and thrombogenicity.

### BRIEF DESCRIPTION OF THE FIGURES

FIG. 1. Multidimensional scaling plot of relationships between pluripotent cell types of mouse and human origin. Mouse: single inner cell mass (mICM) isolated from 3 E3.5 blastocysts; epiblast manually dissected from E6.5,7.5, each duplicates (mEPI); 3 single ESC colonies (WC). Human: 3 single inner cell masses (hICM); epiblast-like cell layer hEPI) manually dissected in duplicate from Nodal expressing EBs (Vallier et al., 2004); 3 single H9 ESC colonies (hESC). All transcriptional profiling was done by the method of Tietjen et al. (2003).

FIG. 2. Biallelic expression of H19 in an H9 sub-line increased as a function of passage number (p); (%) is the fraction of transcripts from “silent” allele; (±) indicates restriction digest.

FIG. 3. Bisulfite methylation analysis of the HI9 DMR in three hESC lines. Each line represents a unique strand of DNA. Circles represent positions and methylation of individual CpGs as follows: filled, methylated; open, unmethylated. The first and last CpG sites are numbered relative to the H19 transcriptional start site. PO1 Primate Core B has now isolated of epiblasts from day 15 nhp embryos. These isolated samples can be placed into culture of frozen in OCT for laser capture experiments, directly dissected and processed for RNA analysis, or even cultured for epiblast-derived embryonic stem cells.

FIG. 4. Sequence of Igf2 ApaI polymorphism (GGACCC (SEQ ID NO: 1) or GGGCCC (SEQ ID NO: 2) Each non-human primate sample was sequenced to determine if a polymorphism is present and the location in the genomic sequence. Samples shown have the sequence GGGCCC (SEQ ID NO: 3), not always in same position (see italicized sequence*, which usually surrounds polymorphism). *aagggcccca gaaatcacag gtgggcacgt cgcgtctacc gccatctccc ttctcacggg aattttcagg gtaaact (SEQ ID NO: 4) BOLD=the site of the polymorphism. IGF2 sample 073 DNA sequence is SEQ ID NO: 5. IGF2 sample 052 DNA sequence is SEQ ID NO: 6. IGF2 sample 022 DNA sequence is SEQ ID NO: 7.

### DETAILED DESCRIPTION

Responsible realization of human embryonic stem cells' (hES cells) biomedical promises is an extremely important research goal. hES cells promise previously unimagined therapies for devastating disorders, and enable unique scientific opportunities to responsibly discover the fundamental mechanisms of healthy human development.

The eventual clinical progression of embryonic stem cell (ESCs) derived tissues for therapeutic applications will require extensive studies of safety and efficacy in both small and large animal system. As such cell based transplantation therapies are likely to consist of allogeneic grafts and the most relevant endpoints for assessment will be comparable allogeneic grafts performed within a relevant species. Because of the extensive evolutionary distance between rodents and humans, and already evident differences between mouse and primate ESCs, there are limits to the use of mouse ESCs to demonstrate safety and efficacy of stem cell-based transplants. Nevertheless, mice (particularly immunodeficient strains) can have great utility in demonstrating the pluripotency of primate ESCs through the development of teratomas induced by transplanting ESCs to ectopic sites. However, it must be kept in mind that even such in vivo assays of pluripotency are surrogates for a more thorough assessment of differentiative capacity and long-term stability of the differentiated state of primate ESC derived cells, their pluripotency, epigenetic status and stability of their differentiated state. This can only be ultimately achieved through even more fundamental assessment of the developmental capacity of pluripotent stem cells as a formal assessment of chimeric studies in embryos, in which contribution and function of ESC derived cells in all primary germ layers and their differentiated derivatives can be definitively assessed.

Based on the foregoing, the present research relies heavily on a non-human primate model as the most relevant model for assessing the therapeutic potential of pluripotent stem cells. While a number of such species are germane options, several considerations point to Rhesus monkeys: their high evolutionary relatedness to humans; the scope and availability of existing breeding stocks (with SNPs valuable to the epigenetic assessments and donor vs. host identification in chimeric tissues); and progression of the Macaque genome project, with imminent production of a Rhesus Genechip. In studying a non-human primate model, a focus on a definitive assessment of pluripotency, capacity for functional differentiation, and epigenetic status in ESCs and EGCs will be undertaken.

As used herein, pluripotency includes the characteristic of immortality and the plasticity to develop into any of the body's cell lineages (perhaps excluding trophectoderm descendents like some extraembryonic and placental tissues). Immortality includes both cellular immortality in vitro and our potentially immortal reproductive cycle in vivo. Fertilized eggs, embryonic cells, inner cell mass cells, embryonic stem cells, embryonic germ cells, primordial germ cells, and embryonic carcinoma cells are endowed with pluripotency. That is, they have the potential to develop into any cell type in vitro and, under special situations, to contribute to any cell lineage (including, at times, the germ cells) when aggregated chimeric embryos are transferred to properly staged surrogates. Pluripotency is best described operationally (e.g., by ectopic transplantation or chimera formation), since only a few of the molecular and cellular constituents have yet been identified. These molecular markers of pluripotency are the subjects of intense international investigation, since regardless of their role in conferring pluripotency, these markers are presently the only keys for distinguishing pluripotent cells from those committed to specific lineages. Molecular markers for pluripotency include Oct314, Nanog, Rex, Sox-2 (although Sox-2 continues to be expressed in early neuroectoderm).

Presently, teratoma formation is used as an in vivo demonstration of pluripotency, since hES cells differentiate into all three germ layers in SCID mice. At early stages of development, the progenitors of-the entire organism-are encompassed-in just three-lineages˜the so called “primary germ layers,” endoderm, ectoderm, and mesoderm. Of these, mesoderm is important as an inducer of organotypic differentiation in the other two, and because it forms key tissues in its own right, for example the musculoskeletal system, which emerges from dorsal components of primary mesoderm, and the blood stem cells, which emerge from the ventral-most aspect of the primary mesoderm; endoderm is important as the epithelial progenitor of the entire system of gut organs, including lungs, pancreas and liver; and ectoderm, is important as the epithelial progenitor of the brain and skin, thus the source of neurons and the supportive cells of the nervous system as well as the multipotent neural crest population. Epigenesis comprises the special circumstances that enable both pluripotency and differentiation. Even with genetically identical twins or inbred mouse strains, the potential for alterations in genomic imprints, cytoplasmic organelle differences and variations in intracellular and extracellular environments are together capable of generating considerable phenotypic diversity.

The goals of the research described herein are: (i) To understand the fundamental biology of pluripotent stem cells during development and differentiation by investigating the NIH-approved hES cell lines and as additional models, deriving and studying nonhuman primate ES cells (nonhuman primate embryonic and germ cell lines) and; (ii) To determine the transplantation potentials of ES cells by non-invasively imaging autografts, allografts, and xenografts into primates and mice, with special attention devoted to focus on the safety of differentiated stem cell transplantation of nhp-ESCs derived from NT embryos, as well as immune tolerance.

### Part I:

### Imaging nhpES Cells In Vitro, in Chimerae, and After Transplantation

Embryonic stem cell pluripotency is most convincingly demonstrated in reaggregated embryos in which the resultant offspring have ES cell contributions to all germ layers and tissues, including the germ line. This has only been achieved using mouse embryonic stem (mES) cells. Overwhelming ethical concerns obviously preclude attempts with human embryonic stem (hES) cells. This project responsibly bridges gaps in the scientific knowledge between mES and hES cells through the generation of chimeric primates with nonhuman primate ES (nhpES) cells—including nhpES cells derived from blastocysts generated after nuclear transfer (NTnhpES cells). Through sophisticated non-invasive imaging, this project responsibly answers crucial questions about: primate ES cell pluripotency during development (Aim I); primate ES cell stability and utility after NT (Aim II); genomic imprinting status after ART and nuclear transfer (Aim III); and HESC and primate ES cell fate after transplantation, especially after SCNT (Aim IV). Nine specific questions are posed in these four Specific Aims: Aim I. To dynamically image nhpES cell contributions in developing primate chimerae: 1.1. Will nhpES cells contribute to chimeric blastocysts, fetuses and healthy offspring? 1.2. With tetraploid embryos (4N), will ES cells contribute primarily to the inner cell mass in chimeric blastocysts, fetus and offspring? Primate chimera are generated in four ways, and the fate of each chimera is followed in vitro during preimplantation development, determining the cellular contributions of the ES and tetraploids to the expanded blastocyst stage after differentially labeling ES or embryos with GFP-transgenes, as well as in utero during fetal development and in the offspring. Aim II. Are ES cells derived after nuclear transfer (NTnhpES cells) developmentally restricted? Two questions are posed: 2.1. Are NTnhpES cell lines stable? 2.2 Will NTnhpES cells contribute to chimeric fetuses and offspring? Aim III. Do Genomic Imprints in NHP Embryos, Fetuses, Placentae, Amniotic Cells and Offspring Differ between ART and NT-Chimerae versus Natural Matings? 3.1. What is the level of DNA methylation in embryos, fetuses, placentae, and offspring after ART, NT, or natural matings? 3.2. What are the parental expressions of selected imprinted genes during NHP preimplantation development? 3.3. What is the DNA methylation status of specific genes in rhesus preimplantation embryos? Aim IV. Dynamic imaging of ES cell fates after transplantation. 4.1. Are NT-derived nhp-ESCs tolerated when transplanted back into the female from which both the somatic cell and the oocyte were obtained? 4.2 Are allogenic ES cells rejected like allografts or do their pre-implantation origins confer immunological privilege? This aim will investigate the tolerance of nhpES in allografts and autografts. Taken together, this project will provide crucial information regarding ES cell pluripotency in nonhuman primates and evaluate the safety of stem cell transplantation in nonhuman primates.

### EXAMPLE I

### 1: To Dynamically Image nhpES Cell Contributions in Developing Primate Chimerae

As stated, question addressed under this aim include: 1.1. Will nhpES cells contribute to chimeric blastocysts, fetuses and healthy offspring? 1.2. With tetraploid embryos (4N), will ES cells contribute primarily to the inner cell mass in chimeric blastocysts, fetus and offspring? This question delves into nonhuman primate chimera generation by performing four sets of reaggregations: fertilized embryo←→fertilized embryo; fertilized embryo←→pluripotent nonhuman primate embryonic stem cells (nhpES cells); fertilized embryos←→tetraploid embryo ♀♂+4N; and tetraploid embryo←→nhpES cell (4N+ES). The fate of each chimera will be followed in vitro during preimplantation development, determining the cellular contributions of the ES cell and tetraploid to the expanded blastocyst stage after differentially labeling ES cells or embryos with GFP-transgenes. Fetal development will also be traced, as well as offspring potential of each chimera after embryo transfer to timed rhesus recipients using MRM noninvasive imaging. Finally, developmental normalcy of embryos, fetuses, placentae and the resultant offspring will be ascertained, along with contribution of nhpES to the offspring.

This first aim studying chimera aggregations in nonhuman primates answers problems regarding cellular lineage determination, the formation of pluripotent ICM cells in blastocyst, and offspring. It will be determined whether nhpES cells contribute to all three somatic cell lineages as well as to the germ-line in reconstructed chimeric embryos in utero and after birth, and in vitro investigating pluripotency markers.

NT involves multiple manipulations and developmental events culminating in the somatic nucleus within the activated and enucleated oocyte. These steps include: a) ‘enucleation’ of meiotic metaphase-II spindle and chromosome complex (SCC); b) somatic cell selection and preparation; c) nuclear transfer or intracytoplasmic nuclear injection (ICNI); d) wound healing and drug recovery from both spindle removal and nuclear introduction, as well as recovery; and e) oocyte activation. In addition, somatic cell preparation and selection has been investigated in several species (Wakayama et al., 1998, Nature 394:369-374; Wilmut, 2002b, Nature 419:583-587; Wilmut et al., 1997, Nature 385:810-813) and electrofusion (‘Dolly’) versus direct injection (‘Honolulu’ or ICNI) are being compared. Wound healing after microinjection, cell fusion and ‘enucleation’ has not been investigated, yet cell sealing in other systems involves new membrane vesicle recruitment by microtubules (Togo et al., 1999, J. Cell Sci 112(Pat 5):719-731). Finally, oocyte activation typically is initiated by the sperm and SCNT has succeeded with DMAP/ionomycin (Navara et al., 1994, Dev Biol 162:29-40), electrofusion (Wilmut et al., 1997, Nature 385:810-813), sperm factor (Perry, 2000, Dev Biol 217:386-393), ethanol (Ng et al., 2004, Development 131:2475-2484), and strontium chloride (Wakayama, 1997, Zygote 5:229-234). Recently discoveries on a novel sperm-specific phospholipase C (PLC-γ) (Cox, et al., 2002, Reproduction 124:611-623; Saunders et al., 2002, Development 129:3533-3544) and electric field induced oscillations (Ducibella, et al., 2002, Dev Biol 250:280) afford additional strategies for more naturally mimicking sperm-induced egg activation.

Transgenic reporters, coupled with pluripotent promoters GFP-Oct4 (Boiani, et al., 2002, Genes Dev 16:1209-1219), and imaged by a new spinning disk form of real-time confocal imaging promise to revolutionize the efficiency of NT. Prior to this work, it was impossible to distinguish a blastocyst generated by fertilization which has nearly every developmental potential, from a cloned blastocyst with perhaps only one-hundredth of the developmental potential. Now, based on our studies and for the first time, morphologically identical blastocysts are readily distinguished by high-resolution, low-light, real-time confocal imaging of GFP-Oct4. Only the ICM cells in the fertilized blastocyst display the GFP-Oct4 signal, whereas GFP-Oct4 is aberrantly expressed throughout the cloned embryo—including in the trophectoderm. These results can be readily extended to primates through the transgenic insertion of the murine or human GFP-Oct4 promoter.

The GFP-Oct4 imaging permits rapid and reliable selection of the most fully reprogrammed nuclei for later transfer. In Aim I, it is proposed to produce chimera by aggregating dynamically labeled nhpES cells with either fertilized or tetraploid embryos and explore ES cell lineage contributions to the resultant chimeric blastocysts. Afterward, nhpES cell-derived chimera will be transferred to recipient rhesus females for noninvasive imaging by MRI during fetal development and the production of healthy offspring. Chimeric mice have been derived after injecting GFP-transfected mouse ES cells into the blastocoel cavity of BALB/c mice. Following embryo transfer, 5/10 (50%) chimeric pups were successfully born as shown by coat color. The nhp embryos have been labeled with GFP insertion by lentivirus.

Primate fetal imaging by MRI permits dynamic assessments of fetal development and viability and ultimately tracking of transplanted undifferentiated and differentiated stem cells labeled with MRI (Aim IV). Selection of the viable chimeric embryos between fertilized or tetraploid blastomeres and nhp-ES cells (Aim I), NT-nhpES cells (Aim II) and nhp-EG cells (Project III) is now possible using the non-destructive real-time GFP-Oct4 imaging typically restricted to only the ICM cells, not the trophectoderm.

As stated, Aim I is designed to dynamically image nhpES cells' contributions in developing primate chimerae. The rationale is that this aim will determine the developmental potentials of nhpES cells in reconstructed chimeric embryos, and investigate nhpES cell behavior and fate after transplantation by non-invasive MRI imaging. Pluripotency in nhpES cells will be demonstrated by rhesus embryo reaggregations with nhpES cells, with the resultant embryos analyzed for ICM/TE distribution in vitro. Finally, rhesus chimera produced with nhpES cells will be transferred to surrogate rhesus females for establishing pregnancies and to generate offspring. Mouse chimera will only be employed for specific control experiments to define critical parameters (transgene reporters, vital dye labeling, optimal concentrations, testing Ultraview confocal imaging characteristics, etc) and quality control purposes.

Question 1.1: Will nhpES cells contribute to chimeric blastocysts, fetuses and healthy offspring? Strategy: to produce rhesus chimeric embryos first using aggregations of ♀♂+♀♂ embryos and then followed by combining nhpES cells with rhesus ♀♂ embryos. An investigation will be done into whether the various chimeric combinations can produce blastocyst in vitro with predicted contributions to the trophectoderm or inner cell mass (ICM). Finally, nhpES cells will be produced with rhesus ♀♂ embryos for embryo transfer to investigate fetal development in utero using MRI imaging and for offspring production.

1.1.A. Production of fertilized NHP embryos from stimulated females by IVF or ICSI is described in 1.1.G. Successful fertilization is confirmed between 5-6 hrs post-insemination by the presence of the second polar body and two pronuclei in the cytoplasm. Zygotes are cultured in fresh CO₂-equilibrated TALP medium until the 2-cell stage. After completion of the first cleavage division (24-28 h post injection), 2-cell embryos are co-cultured in CMRL+10% FCS (Hyclone Laboratories, Inc., Logan, Utah) on Buffalo rat liver cell monolayers (BRL 1442; ATCC, Rockville, Md.) seeded in 100 μl drops overlaid with oil until collection for chimera formation.

1.1.B. GFP expressing transgenic rhesus embryos for chimera production. Rhesus embryos that express GFP are prepared for use in chimera reaggregation experiments (Chan et al., 2001, Science 291:309-312; Chan et al., 2001, Trends Pharmacol Sci 22:214-215). Lentiviral vectors are being pursued because of their high transfection rates and ability to integrate into interphase nuclei. The lentivirus is pseudotyped with vesicular stomatitis virus (VSVG) glycoprotein (Pfeifer et al., 2002, Proc Natl Acad Sci USA 99:2140-2145) and is obtained in collaboration with Dr. Carlos Lois (MIT). The pseudotyped VSVG lentiviral vector is derived as follows: The viral vector backbone used in these experiments is based on a self-inactivating vector. The long terminal repeat (LTR) is required for integration into the cell genome and for transcription of the viral RNA and is derived from HIV-1 with modifications: a deletion of the U3 enhancer plus a 1.2 KB insertion between U3 and R sequences of the 3′ LTR and a deletion of U5 and insertion of human CMV enhancer/promoter in the 5′ LTR. This modification does not affect generation of the viral genome in the producer cell line, but results in self-inactivation of the lentivirus after transduction of the target cell. Once integrated into the transduced target cell, the lentiviral genome is no longer capable of producing packageable viral genome. The PSI sequence is required for packaging genomic RNA into the capsid. The expression of transgene (green fluorescent protein (GFP) gene) is under control of either the human ubiquitin-C promoter or Oct-4 promoter. To increase the level of transcription, the woodchuck hepatitis virus posttranscriptional regulatory element (WPRE) which shown to enhance expression of GFP in transgenic mice is inserted downstream of GFP. The packaging vector contains gag, pol, tat and rev genes that provide the helper functions as well as structural and replication proteins in trans required to produce the lentivirus. The envelope vector contains G glycoprotein gene from Vesicular Stomatitis Virus (VSV-G) as a pseudotyping envelope and allowing production of a high titer lentiviral vector with a significantly broadened host cell range. The three plasmids (1: transgene expression vector contains GFP cDNA, 2: packaging vector contains gag, pol, rev and tat genes and 3: envelope vector contains VSVg gene are co-transfected into 293FT cells (Invitrogen, CA). The 293FT cell is a cell line derived from human embryonic kidney cells that has been immortalized with the early region of adenovirus (EIA and E1B genes) and has been stably transfected with the early region of SV40 to increase transfectability. Genes encoding the structural and other components required for packaging the viral genome are separated onto three plasmids. All three plasmids have been engineered not to contain any regions of homology with each other to prevent undesirable recombination events that could lead to the generation of a replication-competent virus and decrease its potential pathogenicity.

To propagate the virus, the gag, pol, tat and rev genes are supplied in trans by cotransfection of helper plasmids. This helper plasmid contains deletions in LTR, psi sequence, rev responsive element and env, vif, vpu, vpr, and nef coding sequences. The expression of these coding sequences is driven by a CMV promoter, and the polyadenylation signal is derived from SV40 virus. To provide a coat for the virus, the VSVg (vesicular stomatitis virus glycoprotein) envelope gene is provided by cotransfection. The expression of VSV coding sequence is driven by a CMV promoter, and the polyadenylation signal is derived from SV40 virus. The three plasmids (1: transfer vector, 2: gag, pol, rev, tat encoding plasmid and 3: VSVg plasmid) are transfected into 293T cells. Due to the multiple deletions and mutations in the vector backbone and the packaging plasmids, it is highly unlikely to generate a replication competent virus by recombination (coding sequences for env, vpr, vpu, nef; rev responsive element sequence is absent; U3 enhancer from LTR is deleted). 293T is a cell line derived from human embryonic kidney cells that has been immortalized with the early region of adenovirus (E1A and E1B genes) and has been stably transfected with the early region of SV40 to increase transfectability. This cell line does not produce any known infectious agent and does not contain any of the genes that would complement the genetic defects of the viral vector system.

To maximize the success of this research, cell lines producing viruses will be grown exclusively in tissue culture incubators in the BL-2 approved facility at the Pittsburgh Development Center (PDC). Incubators will be appropriately labeled to indicate the type of recombinant virus being generated. To enable concentration of VSVg pseudotyped virus the viral supernatant is collected in a laminar flow hood and placed in ultracentrifugation tubes. The tubes are spun for 1.5 hours at 25000 rpm. In the laminar flow hood, ultracentrifugation tubes are removed from holders, and supernatant disposed of in an appropriate container. Viral pellets are resuspended in PBS and stored frozen at −80 C. All procedures involving plasmid growth, DNA transfection into packaging cells, ultracentrifugation, and viral resuspension will be performed in dedicated BL2 facilities at the PDC. For transecting fertilized rhesus zygotes, the modified Lentil-GFP virus will be used, containing either CMV, Elongation factor 1α, ubiquitin, chicken β-acting, or Oct-4 constitutively active promoters that can express as early as the 2- to 4-cell stages in NHP. The concentrated vector solution is back-loaded into a 6-7 μm sterile microinjection needle (Homage, Inc., Charlottesville, Va.) and a fixed amount deposited into the perivitelline space of pronuclear stage embryos (˜8 hrs post-insemination). After microinjection, the oocytes are returned to culture at 37° C. in 5% CO₂. Beginning at the 2-cell stage of development, confirmation of GFP-expressing embryos is accomplished using attenuated epifluorescent illumination with appropriate filters on an inverted Nikon TE-2000 microscope. 2-cell embryos not expressing are continued in CMRL co-cultured and examined every 24 hrs thereafter to confirm GFP expression.

1.1.C. Embryo Sexing: described, infra.

1.1.D. Derivations of NHP-ESCs. The ICM from ICSI-fertilized expanded blastocysts are isolated by immunosurgery following removal of the zona pellucida with 5 mg/ml Pronase for 2-3 min. Anti-monkey whole serum antibody and guinea pig complement are prepared in 20 μl drops in 35 mm petri dishes overlaid with mineral oil and incubated at 37° C. in 5% CO₂. Zona-free blastocysts are exposed to anti-monkey antiserum for 30 min at 37° C. and washed 3 times with a washing media [1:1 mixture of Dulbecco's modified Eagle's medium (DMEM) and Ham's nutrient mixture F-12 supplemented with 0.1 mM 2-mercaptoethanol, 1% nonessential amino acid, and 15% fetal bovine serum]. The blastocysts are transferred to guinea pig complement and incubated for 15 min at 37° C. in 5% CO₂. The blastocysts are washed three times in washing media and incubated for 15 min at 37° C. in 5% CO₂. The intact ICM is separated from lysed trophectoderm cells by pipetting, and plated on mitomycin C-treated mouse embryonic fibroblasts (MEF). MEF-ICM plates are incubated for 2 days to prevent detachment of the ICM in a 1:1 mixture of Dulbecco's modified Eagle's medium (DMEM) and Ham's nutrient mixture F-12 supplemented with 0.1 mM 2-mercaptoethanol, 1% nonessential amino acid, 1000 U/ml leukemia inhibitory factor and 15% fetal bovine serum. After 7 to 14 days, expanded ICM are dissociated with a fine glass capillary. Colony pieces are transferred to new MEF for expansion.

During the first two months, colonies with stem cell-like morphology are sampled to characterize appropriate known pluripotent markers, karyotypes, and teratoma formation in SCID mice. Pluripotency [Oct-4, SSEA-3, SSEA-4 Tra-1-60, Tra-1-81, Nanog, Rex] markers in NHP embryos are demonstrated in recent publications (Besser, 2004, J Biol Chem 279(43):45076-45084; Booth et al., 2004, Genomics 84:229-238; Mitalipov et al., 2003, Biol Reprod 69:1785-1792; Miyagi et al., 2004, Mol Cell Biol 24:4207-4220; Miyamoto et al., 2004, Stem Cells 22:433-440; Nakatsuji et al., 2002, Scientific World Journal 2:1762-1773; Parisi et al., 2003, J Cell Biol 163:303-314; Thomson et al., 1996, Biol Reprod 55:254-259; Vrana et al., 2003, Proc Natl Acad Sci USA 100 Suppl 1:11911-11916). It is anticipated that 20-25 distinct nhpESC cell lines should be derived, with 5 cell lines actively maintained for investigative work. For chimera reaggregations, the efficiency of early passage stage nhp-ES cells (<20) will be compared with later passage stages (>20) with regards to contribution to trophectoderm or ICM of resultant blastocysts.

1.1.E. Cytogenetic analysis of nhpESC. Karyotype analysis of nhpESCs will be performed by the Cytogenetics Department at Magee Womens Hospital. NHP-ES cells are trypsinized and gently pipetted to give a single cell suspension. A small droplet of the cell suspension is dropped onto precleaned glass slide and placed in an oven at 55-65° C. for 45 minutes. Slides are then incubated in 2×SSC (150 mM NaCl, 15 mM trisodium citrate) at 60-65° C. for 90 minutes followed by rinsing thoroughly in 0.9% w/v NaCl at room temperature. Slides are stained in Trypsin-Giemsa (Bio/medical Specialties, Santa Monica Calif.) solution for 4-6 minutes before transfer to fresh buffer (Ix SSC; twice rinsed) and dried by compressed air. Slides are then mounted and viewed under 100× oil immersion using a Nikon E1000 upright microscope equipped with high numerical aperture objectives. Chromosome spreads are captured digitally using an ORCA Cooled CCD camera (Hamamatsu, N.J.).

1.1.F. Teratoma Formation in SCID Mice: To assess the potential of the stem cells to contribute to all three-germ lineages, all NHP derived stem cells will be assessed for teratoma formation in SCID mice. Formation of teratomas with cell lineages derived from ectoderm, mesoderm, and endoderm will be considered proof of pluripotency, a prerequisite requirement prior to using a newly established nhpESC cell line for chimera reaggregation experiments.

1.1.G. NHP-ESC labeling. As for intact embryos, a stable colony of GFP-expressing nhp-ES cell lines will be established prior to their use in chimera reaggregation experiments. GFP-reporters for ES cell tracking are proposed as the best and longest lasting reliable marker. Two different approaches will be followed towards the generation of transgenic non-human primate embryonic stem cells using GFP plasmids or retroviral vectors containing GFP. The first involves GFP-lentiviral transfection of rhesus zygotes for the production of GFP-expressing embryos that are cultured to the expanded blastocyst stage prior to the derivation of NHP-ES cells, as described in 1.1.D. The second method involves either direct lentiviral transfection or Lipofectamine transfection of previously derived ES cells. Lipofectamine transfection of nhpESC is performed according to (Vallier et al., 2004, Stem Cells 22:2-11). Positive colonies are expanded over the next month to attain a population of stable transfected cells as described in 1.1.D.

1.1.H. Chimera formation: For ♀♂+♀♂ embryo chimera formation, an aggregation plate is prepared using a sterile Darning needle to create 6 depressions within 30 ul droplets of CMRL medium overlaid with mineral oil (Nagy et al., 2003, Manipulating the Mouse Embryo: A Laboratory Manual. Cold Spring Harbor Laboratory Press, Cold Spring Harbor, N.Y.). After 8-cell embryo biopsy is performed, the zona pellucida is removed with 0.5% pronase (5-8 min at 37° C.) and then rinsed twice in culture media. Two zona-free embryos, one distinctly labeled green after GFP-expression, are transferred into a single depression well. After all fertilized embryos reaggregations are preformed, the plate is gently swirled to bring the pairs into close contact before returning to culture at 37° C. in 5% CO₂. For combining nhpES cells with rhesus ♀♂ embryos, two methods will be investigated. First, biopsied embryos are microinjected with 8-10 GFP expressing nhpES cells into the space vacated by blastomere withdrawal and the chimera's returned to BRL co-culture for embryonic development. A second method employs reaggregation of fertilized embryos with small GFP-expressing colonies (10-15 cells). Rhesus 8-cell embryos are prepared and a small clump of transfected ES cells introduced into the depression wells. After all reaggregations are completed, the plate is gently rotated to bring the fertilized embryos in close contact with the ES cells before returning to culture. All plates are checked the following day for cellular reaggregation and subsequent embryonic development. Collected embryos are kept at 37° C. in a 5% CO₂ until tetraploid formation or chimera aggregation as described above.

1.1.I. Dynamic imaging of cell tracer-labeled chimeric embryos by a spinning microlens array confocal microscopy (UltraView; Perkin-Elmer, Boston, Mass.) is performed as described herein.

1.1.J. Embryo transfer of ♀♂+♀♂ and nhpES cells+♀♂ rhesus chimeras into timed recipients for pregnancy establishment is described elsewhere herein. Chimera reaggregations will be made with two distinct embryos of separate parental lineage typically with GFP-expressing cells for later analysis of tissue contribution as describe in 1.1.L.

1.1.K. Noninvasive in utero imaging by high definition ultrasound (U/S) and MRI, and Amniocentesis. Conventional ultrasound/Doppler imaging is performed at defined post-implantation intervals to document attainment of normal in utero parameters, i.e., 40-44 days gestation, a differentiated tissue mass showing body axis, limb-buds, the developing brain, and liver; at 60-65 days gestation, the developing brain and many internal organs are apparent (e.g., heart, liver, spleen, intestines, bladder, etc.). The placenta is also clearly detected. Specific measures of mean gestational sac size, yolk sac diameter, greatest length, and embryonic heart rates are collected to approximate the gestational age of the conceptus. These are compared to similar measurements made from IVF and natural pregnancies (Tarantal et al., 1988, Am J Primat 15:309-323). However, to obtain three-dimensional (3D) non-invasive views into optically opaque tissues with exquisite sensitivity will require magnetic resonance imaging (MRI). Specially, MRI will provide knowledge about the internal structure and function of intact living systems in the fetus, especially permitting high-resolution 3D imaging to resolve fine details of the anatomy of the developing embryo and placenta. Quantitative imaging methods, such as diffusion tensor imaging, provide information about developmental changes at the cellular and microstructure level at near-cellular resolution. Any tissue morphological changes, progression of disease states, and biochemical changes will be visualized with MR microscopy. Using MR microscopy methods it is possible to routinely produce 3D images with an isotropic resolution of less than 50 micrometers (Ahrens et al., 2002, Progress in Nuclear Magnetic Resonance Spectroscopy 40:275-306; Jacobs et al., 1999, Comput Med Imaging Graph 23:15-24). Exposure to MRI magnetic fields was not teratogenic in mice, as shown by a sensitive assay for birth defects (Heinrichs et al., 1988, Magn Reson Imaging 6:305-313).

1.1.L. Mt-DNA and RT-PCR Analysis: To determine whether chimera blastomeres have contributed to either extra-embryonic or fetal tissue, we will analyze various biopsied tissues following birth of offspring (hair, buccal cells, skin, muscle, blood, umbilical cord and placenta). For potentially transgenic offspring, samples will first be screened for the presence of the transgene using standard PCR methodology. Samples positive for transgene presence will be further analyzed using RT-PCR to determine if the GFP transgene is being expressed (Somerset et al., 1998, J Clin Endocrinol Metab 83:1400-1401) as well as examined by a brief exposure to attenuated epifluorescent illumination to confirm the extent of mosaic expression of tissues using appropriate fluorescein filters. Analysis of chimera contribution to tissues will be carried out by first using mitochondrial DNA (mtDNA) fingerprinting techniques to screen for polymorphisms present between animals within the Pittsburgh colony (Dyke et al., 1990, Prog Clin Biol Res 344:563-574; Hewitson et al., 2002, Reprod Biomed Online 5:50-55; Holmes et al., 1986, Alcohol Clin Exp Res 10:623-630; Khan, 1987, Genetica 73:25-36). As recently described by (St John et al., 2004, Genetics 167:897-905), nested PCR followed by automated sequencing of portions of the mtDNA displacement loop (D-loop) region will be used to screen polymorphism among animals within the Pittsburgh rhesus colony. Specifically, we will amplify the hypervariable regions (hv1 and hv2) of the D-loop of the mitochondrial genome from blood and or tissue samples (skin biopsy). The amplimers will then be sequenced using standard automated sequencing methods (Hopgood et al., 1992, Biotechniques 13:82-92). Sequences will be analyzed for unique polymorphisms using a sequence analysis package such as the freely available ClustalW (www.ebi.ac.uk/clustalw). Real-Time PCR primers and probes will be designed based upon each animal's unique polymorphisms. Multiplexed Real-Time PCR will be performed on DNA samples obtained from chimeric offspring using primers specific to each parent of origin. Overall levels of chimerism will be determined as a percentage of the mtDNA present from each parent of origin as determined by the multiplexed Real-Time PCR.

1.1.M. Data collection, analysis, and expected results: We estimate that 50-60 blastocyst will be needed to derive answers on ♀♂×♀♂ chimera and perhaps as many as 100 blastocyst for ♀♂×nhpESC chimera to determine-allocation to the ICM or trophectoderm following in vitro culture. The numbers of ♀♂×nhpESC chimera reflects our hope to compare early versus late passage nhp-ESC with regards to efficiency to contribute to various lineages in the resultant blastocysts. Variability may be extensive giving factors of embryo quality, success of aggregations, and ability of zona-free embryo pairs to develop in vitro, and each parameter is carefully recorded and analyzed. We have proposed using two methods for producing chimera—one involves direct cell injection into biopsied embryos while the other employs reaggregation of stem cells with early-fertilized embryos in shallow depression wells in the culture dish.

TABLE I
DATA COLLECTION AND ANALYSIS OF
NHP-ESC & NT-NHP-ESC CHIMERAE
[Compared with Chimeric Controls Generated by ♀♂-Blastomeres or ICM-Chimera]

| Morulae | Blastocysts | | Day 35 Fetus | Amnios | Offspring | Placenta | Epiblasts |
|---|---|---|---|---|---|---|---|
| NhpESC's | NhpESC or | PRE- | Complete PCR | Normal | At birth, cord | Immuno- | If no |
| or | NTnhpE | Implt | and | viable | blood, | histo- | chimeric |
| NTnhpE | SC | Left | histology | pregnan | skin, | chemistry & | nhpESC |
| SCs | incorporate | | of organ, | cies, | urogenita | placental | fetus, |
| adhere, | with | | tissues | ascertained | I cells | cultures, | then |
| form | ICM | | Especially | by | (from | in | epiblasts |
| junctions, | preference; | | germs | Part V, | urine- | addition | collected |
| viable & | GFP- | | cells in | A with | soaked | to | day |
| compact. | Oct4 | POST- | fetal testis | U/S & | diapers). | analyses | 15. |
| GFP- | expression | Impl → | or ovaries. | MRI, on- | Analyzed | in #3 | nhpESC |
| Oct4 | ICM | | Cultures. | going. | without | & #4 | chimera |
| expression. | restric. | | | Non- | endange | | cell |
| Retain | Pluripotency | | Strategy: | viable | ring fetal | | allocation, |
| pluripotency | markers; | | If | fetus | viability. | | versus |
| markers; | proliferation & | | chimeric | collected | Fluorescence | | ♀♂ |
| proliferation & | replication; | | →Amnios, | swiftly | endoscopy, | | chimera, |
| replicatition | Verify | | Offspring, | avoids | biopsies | | ICM- |
| detected. | ESC | | Placenta | degradation; | later | | chim, & |
| | viability | | | differentiated | biopsies | | somatic |
| | by rederive. | | If not → | cell/tissue | later. | | cell |
| | | | examine | cultures; | Sperm | | chimera |
| | | | Earlier | full | by 9 mo.; | | for |
| | | | Epiblast. | pathology; | Eggs by | | survival, |
| | | | | #3 | ovarian | | proliferation, |
| | | | | molec. | biopsy. | | engraftment |
| | | | | Studies. | | | |

Using our sophisticated imaging capability (Project IV) and the ability to differentially label embryo or nhpES cells with GFP-expressing blastomere, we can determine by fluorescence the contribution to mural or polar trophectoderm versus the inner cell mass in blastocysts. We can determine cell position and numbers based on 3D reconstructions for each morula or blastocyst derived by chimera reaggregation, and we will repeat a minimum of 5 times to detect trends. For in utero fetal development, we will use MRI and U/S on paired control and chimeric concepti for detecting defects in cranial, somite, and limb development after ET. Additionally, amniocentesis and CVS will be collected once to confirm if the developing fetus is chimeric using mtDNA and PCR analysis. For offspring, proof of definitive integration will be accomplished by performing mt-DNA analysis on selected tissue types, including placenta, extraembryonic membranes, skin, hair, muscle, and intestine (biopsy). These tests will detect cell fate and contribution to a specific tissue type to demonstrate chimeric contribution to resultant offspring. Additionally, real-time PCR for mt-DNA will quantitative the ratios of cellular contributions from each donor cell source. We expect rhesus ♀♂+♀♂ embryo chimera will form blastocyst with proper ICM and trophectoderm lineages. We anticipate that rhesus ES cell reaggregations with fertilized embryos will produce chimeric blastocyst in vitro, with contributions of the ES cells to the ICM exclusively when reaggregated by either cell injection or zona-free co-culture. We expect that xenotransplantation into SCID mice might prove to be a more useful tool in determining tissue development extent in ES cell-♀♂ chimera because early concepti recovery is possible. Details of SCID mice xenotransplantation are provided in Part VI. Our previous observations indicate that FISH can be completed within 3 hr after isolation of embryonic blastomeres, a timeframe that will not have a detrimental effect on aggregating chimeras. Effect of disaggregation and reaggregation on the viability of newly created chimeras will be compared with the viability of non-manipulated controls. Chimeras will be culture, their development monitored, and their normalcy confirmed by cytogenetic analysis prior to initiation of EI′ into recipient females, SCID mice, or isolation of ES cells. We have already demonstrated that embryo reaggregation can result in viable rhesus offspring. We expect that chimeras created from transgene blastomeres will retain gene expression throughout preimplantation development and we are hopeful that GFP-transgene presence and expression will be maintained in the majority of fetal and newborn tissues. It is likely that embryo transfer of transfected chimeras will result not only in higher rates of transgenic offspring but also in offspring that will demonstrate germ line transmission of the transgene, as recently demonstrated in the birth of ANDi (Chan et al., 2001, Science 291:309-312; Chan et al., 2001, Trends Pharmacol Sci 22:214-215).

1.2.A. Production of rhesus fertilized and GFP-expressing transgenic embryos is described 1.1.A and 1.1.B.

1.2.B. Production of tetraploid intraspecific NHP will be performed by a modified technique of (Nagy et al., 2003, Manipulating the Mouse Embryo: A Laboratory Manual. Cold Spring Harbor Laboratory Press, Cold Spring Harbor, N.Y.). Basically, two-cell embryos grown in vitro (Project V) are placed into an electrode fusion chamber (500 μm gap; GSS-250, BLS, Hungary) in fusion media consisting of 0.25 M sorbitol, 0.10 M mM calcium acetate, 0.10 M magnesium acetate, and 0.5 mg/ml BSA (Fatty-acid Free; Sigma), pH 7.2 (265 mOsm). After equilibration for 15 min, an alignment pulse of 2 V is applied to correctly orient the 2-cell embryos relative to the electric field before applying the fusion current (60 V; 35 μs) to fuse the two blastomeres. Following fusion, embryos are washed and placed in CMRL co-culture medium. Blastomere fusion is confirmed 20-30 min later by inverted HMC optics at 20×.

1.2.C. GFP expressing tetraploid embryos will be performed as in 1.1.B.

1.2.D. NHP-ES cells derivation, cytogenetic analysis, and transfection is described in 1.1.D, 1.I.E, & 1.1.G respectively.

1.2.E. Chimera Reaggregation of tetraploid embryos with fertilized embryos or ES cells is performed as in 1.1.H.

1.2.F. Analysis of cellular lineage contribution of tetraploid+fertilized embryo or tetraploid+ES cell reaggregations at the blastocyst stage will be performed as in 1.1.I.

1.2.G Embryo transfer is performed as in 1.1.J and Part V.

1.2.H. Monitoring post-implantation fetal development is performed by U/S and MRI analysis as in 1.1.K and Part IV.

1.2.I. Determining parental lineage of chimera implants or offspring is performed as described in 1.1.L.

1.2.J. Data collection, analysis and expected results are fully described in 1.1.M. If rhesus chimeras behave as in the mouse system, we anticipate that tetraploid reaggregations with fertilized embryos will produce chimeric blastocysts in vitro and that tetraploid blastomeres will contribute to the trophectoderm exclusively in the chimeric blastocyst. These chimerae are expected to implant following ET and demonstrate axis development. We anticipate that tetraploid cells will largely be restricted to the placenta and extra-embryonic membranes. We also expect to demonstrate nhpES cell segregation largely to the inner cell mass while the tetraploid cells will largely be restricted to the mural and polar trophectoderm in successful chimera. However, some ES cells may contribute to the trophectoderm (Xu, 2002) and we plan to estimate this contribution in the fully expanded blastocyst. In the developing fetus and live offspring, the nhpES cells are expected to contribute to all three tissue lineages (endoderm, ectoderm, and mesoderm) as well as to germ cells. All chimeric concepti will be monitored for defects in cranial, somite, and limb development after ET by high definition ultrasound and MRI analysis. GFP transgene infected embryos will be examined by a brief exposure to attenuated epifluorescent illumination to confirm the extent of mosaic expression using appropriate fluorescein filters. All confirmed fluorescent embryos will be cultured separately or transferred to proper stage females for pregnancy establishment and noninvasive imaging. Control embryos will be exposed to similar levels of fluorescent light to monitor any long-term effects of mercury light exposure on developmental progression and pregnancy establishment. The integration of the GFP transgene into various tissues will be determined by PCR analysis as described in 1.1.L. We estimate that 50-60 chimeric blastocyst will be needed for ♀♂×4N tetraploid chimera allocation to the ICM or trophectoderm following in vitro culture. We also anticipate producing 100 chimeras for nhpESC's×4N embryo chimera since we need to determine the number and passage of ES cells to aggregate with the host embryos. Initially, 10-15 nhpESC cells for aggregation experiments will be used, as this represents the estimated number of primitive ectoderm cells present in NHP ICMs.

1.2.K. Potential difficulties and limitations are similar to those described in 1.1.N. In addition, 4N embryo formation by electrofusion may be complicated by low efficiency or embryo lysis. An alternative method to produce 4N rhesus embryos is incubating embryos at the late 2-cell stage (38-44 hrs post ICSI) in 20 μM cytochalasin B, a reversible microfilament inhibitor that blocks cytokinesis without interrupting karyokinesis, for 12 hours. After inhibiting one round of cell division, the cytochalasin is removed by washing in culture medium and continuing development in CMRL. Control embryos will be run in parallel using similar DMSO (the cytochalasin solvent) concentrations, which never exceed 0.5%. Based on preliminary evidence, we do not anticipate difficulties in generating tetraploid+fertilized chimeric rhesus embryos or culture in vitro. We have also demonstrated the feasibility of producing live transgenic and reaggregated NHPs.

### EXAMPLE I

### 2 Are nhpES Cells Derived After NT Developmentally Restricted?

Rationale: SCNT may overcome immune rejection problems since the major histocompatibility antigens (MHCs) are nuclear transcripts. Our recent findings suggest that meiotic spindle extraction during SCNT in primates depletes the oocyte of vital microtubule motors preventing normal development. Because primate SCNT embryos display aneuploidy, techniques have been adopted from the successful report of stem cell derivation from cloned human blastocyst and methods are now being optimized in nonhuman primates in collaboration with newly recruited scientists from Seoul National University (Eul-Soon Park, Miss.), MizMedi Hospital and Hangyang University (Jong-Hyuk Park, PhD). We have now confirmed optimization of these techniques and are now deriving NT-nhpESC and verifying euploidy through cytogenetic and molecular approaches. ES cells will be isolated from SCNT-derived blastocysts and stable cell lines produced. These ES cells will be investigated in reaggregation experiments with fertilized and tetraploid embryos for cellular lineage contributions during blastocyst formation. Later, NTnhpES cell-derived chimeric embryos will be transferred for pregnancy and offspring production.

Thus, this second aim focuses on deriving ES cells from blastocysts generated by NT and addresses two important goals. First, NT will provide important advances to the generation of normal embryos for the production of genetically identical nonhuman primates. Secondly, the production of euploid embryos will facilitate deriving NT ES cells that can be used for chimera production, attempts that, if successful, will quickly advance therapeutic cloning technology. This latter question explores whether NTnhpES cells contribute to chimeric blastocysts, fetuses, and offspring when reaggregated with fertilized embryos (NTnhpES+♀♂) or with tetraploid embryos (NTnhpES+4N). In so doing, we will determine if these NTnhpES cells have the same pluripotency as in mice (Wakayama et al., 1997, Zygote 5:229-234). Their properties in vitro, in utero in chimeric embryos, and in vivo after transplantation will provide information relevant for more informed evaluations of this strategy in overcoming immune rejection for stem cell medicine.

Aim II describes exciting new strategies for deriving NTnhpES (embryonic stem cells from blastocysts generated by nuclear transfer) which we can confirm in NHPs (Simerly et al., 2004, Dev Biol 276:237-252). Among the advances reported included: a) enucleation by squeezing out the SCC instead of pipette aspiration; b) enucleation of pre-metaphase-II SCC, i.e. at telophase-I or prometaphase-II just after first polar body extrusion, accomplished by live imaging of the meiotic spindle with orientation independent polarization optics. This also permits the selection and transfer of mitotic spindles from both somatic and embryonic stem cells, a technique used in successful production of cloned rat offspring (Zhou et al., 2003, Science Express 10:1126); c) use of the patients own cumulus cells as the donor source for NT; d) use of sequential media optimized first in human ART clinics for production of fertilized human blastocysts; e) use of fructose in place of glucose as an energy source; and f) synthetic serum replacer. In preliminary experiments in non-human primates, adoption of these discoveries has improved SCNT development to the blastocyst stage. Chiefly, enucleation of the pre-metaphase-II SCC permits retention of critical centrosomal and first mitotic spindle assembly proteins than by pipette aspiration. Furthermore, application of an electrical pulse for simultaneous nuclear fusion and cytoplast activation avoids use of harsh chemical activation treatments, resulting in better embryo development to the blastocyst stage. Of the three SCNT blastocyst produced by simultaneous fusion/activation, two had identifiable inner cell mass [ICM] cells following immunosurgery for NT-nhp-ES cell derivations.

In Aim 2, two questions are posed: 2.1. Are NTnhpES cell lines stable? 2.2. Will NTnhpES cells contribute to chimeric fetuses and offspring? The strategy includes constructing two different chimeras, NTnhpES cells←→fertilized embryos and NTnhpES cells←→tetraploid embryos, for investigating in vitro development to the expanded blastocyst stage and determining cellular fates following differentially labeling of NTnphES cells and embryos with transgenes. We will also determine fetal and offspring outcomes following embryo transfers.

Question 2.1. Are NTnhpES cell lines stable? Strategy: This question investigates the adoption on the report of cloned. Successful SCNT and development of euploid embryos to the blastocyst stage will serve as the starting material from which NTnhpES cells will be derived.

2.1.A. Enucleation: Before enucleation, cumulus cells are removed mechanically by pipetting in TALP-Hepes containing 0.3% BSA and 1 mg/ml hyaluronidase. Oocyte aspiration is performed as described in Part V at 27-28 hrs post-hCG and maturing oocytes that have not elicited the first polar body selected for nuclear transfer experiments. Oocytes are cultured in CMRL medium at 37° C. in 5% CO₂ and observed every 30 minutes for signs of first polar elicitation, confirmed by dynamic imaging of the SCC in unfertilized NHP oocytes using polarization optics (SpindleView™; CRI, Cambridge, Mass.). The forming second meiotic spindle is seen as a bright, birefringent bipolar—structure against the dark cytoplasm. ‘Squish’ enucleation of oocytes is performed as follows: pre-metaphase-11 stage oocytes are placed into TALP-Hepes media containing 0.3% BSA and 7.5 μg/ml cytochalasin B overlaid by mineral oil. The zona pellucida is partially dissected with a fine glass needle to make a slit near the forming first polar body. Next, the first polar body and underlying cytoplasm containing the SCC is extruded by gently squeezing them with the same needle. Enucleation is confirmed by visualizing the karyoplast stained with Hoechst 33342 (Sigma.) under attenuated UV illumination to confirm removal of the SCC.

2.1.B. Preparation of somatic donor cells for SCNT. A small pieces of skin from the same female selected for oocyte—donation is collected from a punch biopsy near the shoulder blade, washed three times in Dulbecco's phosphate buffered saline (DPBS, Life Technologies), and then digested in 0.25% (v/v) trypsin-EDTA (Life Technologies) solution for 1 h at 37° C. After washing three times in Ca²⁺- and Mg²⁺-free DPBS by centrifugation at 43×g for 2 min, the pellet is resuspended in Dulbecco's modified Eagle's medium (DMEM, Life Technologies) supplemented with 10% FBS, 1 mM glutamine, 25 mM NaHCO₃ and 1% nonessential amino acid solution and the cells are plated in 75 mm culture flask. The cells are subcultured 2-4 times every 2-3 days before cryopreserving at −196° C. until needed. Prior to nuclear transfer experiments, thawed cells are grown to confluency by culturing for 2-3 days in the DMEM. Individual cells are collected by trypsinization as above for 30 sec, washed by low speed centrifugation, and used within two hours for SCNT.

2.1.C. SCNT. Enucleated oocytes are washed 3 times TALP-Hepes containing 0.3% BSA and placed in a 50 μl drop of TALP-Hepes containing 0.3% BSA in a micromanipulation chamber containing donor cells. Donor cells are aspirated into a microinjection pipette (internal diameter, 20-25 μm; Humagen, Inc) and introduced through the same slit in the zona pellucida made during enucleation step. The cell is wedged between the zona and the cytoplast membrane to facilitate close membrane contact. Couplets are equilibrated in fusion medium [0.28 M mannitol (Sigma Co.), 0.5 mM HEPES, and 0.05% fatty acid-free BSA with 0.1 mM magnesium chloride] at room temperature in a chamber consisting of two stainless steel electrodes 3.3 mm apart (BTX, San Diego, USA) prior to electrical fusion using two DC pluses of 1.78 kV/cm for 15 μs each, delivered by BTX Electro-cell Manipulator 2001. Couplets are rinsed in TALP-Hepes, and then incubated for 2 hrs in G1.2 (Vitrolife, Inc, Englewood, Colo.) at 37°; 5% CO₂. Electrofusion is confirmed by inverted HMC optics within 30 min.

2.1.D Activation of SCNT Constructs: After 2-hrs for reprogramming, reconstructed oocytes are activated in 5 μM ionomycin (Sigma) for 4 min in TALP-Hepes. After rinsing, the constructs are placed in G1.2 medium containing 2 mM 6-DMAP (Sigma) and incubated at 37°, 5% CO₂ 5% O₂ and 90% N2 for 4 hrs.

2.1 E. Culture of Activated SCNT Constructs: Activated oocytes are washed with TALP-Hepes and cultured in 20 μ1 drops of G 1.2 medium at 37°, 5% CO₂, 5% O₂ and 90% N₂ for 48 hrs. On the third day of culture, cleaving embryos are either selected for transfer to timed female recipients or transferred to the hmSOFaa (Vitrolife, Inc) media and cultured for another 7 days until the blastocyst stage for derivation of NT-nhp-ES cells as described below.

2.1.F. Immunosurgical isolation of NT-ES cells using anti-monkey IgG serum and guinea pig complement will be performed as described in 1.1.D.

2.1.G. NT blastocyst imaging for pluripotency markers: The zona pellucida in SCNT blastocysts will be removed with 0.5% pronase and the blastocyst recovered for 30 min in hmSOFaa medium. After attaching zona-free blastocyst to polylysine-coated cover slips in serum-free hmSOFaa, the embryos are fixed in 2% formaldehyde in hmSOFaa for Ihr or in absolute methanol (−20° C.; 20 min) diluted directly into the well. Methanol addition is performed while watching under the dissecting scope to ensure blastocysts are not displaced. After fixation, the embryos are rinsed overnight in phosphate-buffered saline (PBS) containing 0.1% Triton X-100 detergent (PBS-TX) to permeabilize the blastocyst. Primary antibody staining for formaldehyde fixed blastocysts includes SSEA-1 and SSEA-3 or-4 (1:5 dilution; Developmental Hybridoma Bank, Iowa City, Iowa) as well as fluorescein-labeled Tra 1-60 or Tra 1-81 (1:10; Santa Cruz Biotechnology, Inc, Santa Cruz, Calif.). Anti-Oct-4 antibody (Santa Cruz Biotechnology) is used 1:5 on methanol fixed blastocyst. All primary antibodies are applied for Ihr at 37° C. After a 30 min rinse in PBS-TX, unconjugated primary antibodies are detected with a 1:100 dilution of Alexa-conjugated goat anti-mouse IgG (for SSEA-3 or-4, Oct-4) or goat anti-mouse IgM (SSEA-1) applied for 1 hr at 37° C. After a final rinse in PBS-TX, Hoechst 33342 (10 μg/ml; Sigma) and Toto-3 (1 μM; Molecular Probes) are applied for 10 minutes to label DNA for detection by conventional and confocal microscopy, respectively.

2.1.H. Cytogenetic analysis of cultured NT-ES cells by FISH analysis is in described 1.1.E and the Imaging Part IV. Cytogenetic analysis is performed once every six months to ensure genetic stability within the isolated NT-ES cells.

2.1.I. Testingpluripotent markers in derived NT-ES cells will be performed on methanol or formaldehyde fixed colonies after transfer to gelatin-coated cover slips for 24 hours. Immunocytochemistry will be performed using appropriate molecular markers for inner cell mass cells [Oct 4; SSEA-4, Tra 1-60, Tra 1-81] and trophectoderm lineages (SSEA-1) as described in 2.1.G.

2.1.K. Teratoma Formation in SCID Mice is described in 1.1.F and Part VI.

2.1 J. Differential labeling and apoptosis of blastocysts: We will explore the normalcy of NT-cloned and control parthenogenetically-activated blastocysts by ascertaining total ICM versus trophectoderm cells and the percentage of apoptosis present. Control and cloned blastocyst are incubated in rabbit anti-rhesus monkey spleen antiserum in hmSOFaa medium for 30 min at 37° C. After three washes in TALP-Hepes, blastocysts are incubated in guinea pig complement (Sigma) diluted 1:10 in CMRL medium, containing 20 mg/ml propid um iodide (PI) and incubated for 10-15 min at 37° C. This activates the complement cascade rendering the trophectoderm cells permeable to PI. After 10 min, blastocysts are washed in TALP-Hepes and transferred to hmSOFaa medium containing 5 μM Hoechst and cultured for at least 30 min at 37° C. before being mounted on microscope slides in a small drop of water-based antifade solution (Slow-Fade, Molecular Probes, Eugene, Oreg.) underneath a cover slip. Blastocysts are examined by epifluorescence as describe in Part IV. The TE nuclei, labeled with both PI and Hoechst are pink; the ICM nuclei labeled with Hoechst are blue. The numbers of TE and ICM cells in blastocysts derived from NT-clones are compared with ionomycin/DMAP parthenogenetic control embryos. Staining for apoptotic cells is accomplished using a terminal deoxynucleotidyl transferase (TdT) mediated dUTP nick-end labeling (TUNEL) assay kit (Boehringer Mannheim, USA; (Chan et al., 2000a, Curr Top Dev Biol 50:89-102). The complete fixation and TUNEL assay is performed in Terasaki dishes (Greiner, Round Lake, Fla.). Zona-free blastocysts are first fixed in 2% formaldehyde (pH 7.4) for 30 min, rinsed in PBS, then permeabilized in PBS with 0.1% Triton X-100 and 0.1% NaCitrate solution at 4° C. for 2 min. The broken DNA ends of the embryonic cells are labeled with TdT and fluorescein-dUTP for 60 min at 37° C. The blastocysts are counter-stained with 1 μg/ml Hoechst to visualize total DNA. The blastocysts are then mounted onto glass slides using Vectashield. To prevent pressure on the blastocysts and to retain their three-dimensional structure, two coverglass spacers (170 μm height, i.e. >130-150 μm rhesus embryo diameter) are placed beneath the cover slip alongside the droplet of Vectashield. Mouse blastocysts pretreated with DNase I (5 U/50 μl PBS, Roche Diagnostic Corp., Indianapolis, Ind.) will be used as positive controls for the TUNEL assay and blastocysts in which terminal TdT transferase is omitted will serve as negative controls.

2.1.L. Data collection and analysis: SCNT-derived blastocyst and derived NT-ES cells are examined using both conventional immunofluorescence and laser-scanning confocal microscopy as described in Imaging Part IV. Conventional fluorescence microscopy is performed first using a Nikon E1000 epifluorescent microscope with high numerical aperture objectives, since photobleaching is negligible. Data are first collected by a chilled CCD camera (Hamamatsu, Japan) using Metamorph software (Universal Imaging, West Chester, Pa.). Laser-scanning confocal microscopy is performed next using a Leica TCS-SP2 equipped with Argon and dual Helium-Neon lasers for the simultaneous excitation of fluorescein, rhodamine, and Cy-5. For NT-blastocyst, we will record the staining profile of the inner cell mass cells as well as the polar and mural trophectoderm. We will also determine the ratio of each pluripotent marker by comparing the staining profile of each probe to the total number of cells labeled by the DNA stains. Estimates of the number of aneuploid cells observed in each blastocyst will also be determined. For derived NT-ES cells, colony morphology as well as the staining characteristics of each pluripotent marker will be recorded. NT-ES cells that demonstrate Oct 4 nuclear staining and plasma membrane SSEA-4 but are negative for SSEA-1 will be considered undifferentiated.

2.1.M. Expected results: We have already observed success for producing NT-derived blastocysts with low aneuploidy rates and proper organization with regards to ICM and trophectoderm cellular lineages. Further, if like mice, we might anticipate that Oct-4, SSEA-4, Tra 1-60, Tra 1-81 will be correctly expressed within the ICM of only some NT blastocysts (Project IV). If so, we plan to correlate Oct4 expression profiles dynamically using GFP expression under the Oct-4 promoter as recently described in rodent studies (Boiani et al., 2002) to help select the healthiest NT blastocysts for NT nhpES cell derivations. We predict that SSEA-1 will label the trophectoderm cell lineages. We also anticipate that successfully derived NT-ES will remain undifferentiated in culture and express appropriate molecular markers of pluripotency in the ICM and trophectoderm. Cytogenetic analysis will confirm that derived NT-ESCs remain chromosomally stable after culturing. We anticipate that we will derive one successful NT-nhpES cell line for every 25 cloned blastocyst produced after SCNT. Oct-4 expression in NHP embryos has been recently reported by (Pau and Wolf, 2004). We predict that we will need 25-30 rhesus control or cloned blastocysts for investigating pluripotent markers. For differential labeling and apoptosis, we predict that we will need an additional 24 control and 24 NT-derived blastocyst for determining normalcy of NT.

Question 2.2. Will NTnhpES cells contribute to chimeric fetuses and offspring?

2.2.A. Production of fertilized rhesus embryos is described in Part V and 1.1.A.

2.2.B. Production of NT-ES cells is described in 2.1.H.

2.2.C. GFP transfection of zygotes and NT-ES cells is described in 1.1.B and 1.1.G.

2.2.D. Reaggregation of NT-ES cells with fertilized or tetraploid embryos as described in 1.1.H and 1.2.B.

2.2.E. Cytogenetic determination of chimera embryos by FISH analysis is described in 1.1.E.

2.2. F. Analysis of cellular lineage contribution of GFP expressing NT-ES cell reaggregations at the blastocyst stage is described in 1.1.L

2.2.G. Embryo transfer of NT-ES cell+fertilized or tetraploid embryos is described in Part V and 1.1 J.

2.2.H. Xenotransplantation of chimera embryos into SOD mice is described in 1.1.F and Part VI.

2.2.I. Monitoring post-implantation fetal development is described by Ultrasound and MRI analysis in Imaging Part IV arid 1.1.K.

2.2J. Determining parental lineage of chimera implants or established pregnancies is performed as described in 1.2.1.

2.2.K. Analysis of mtDNA and RT-PCR will be performed as described in 1.1.L.

2.2.L. Data collection and analysis is similar to 1.1.M and 1.2.M.

2.2.M. Expected results: We anticipate that NT-ES cell reaggregations with fertilized or tetraploid embryos will produce viable chimeric blastocysts in vitro. We expect to demonstrate that NT-ES cells will contribute predominately to the ICM in either fertilized or tetraploid embryos by either cell injection or zona-free co-culture. To determine both presence or absence of transgenes and whether NT-ES cell chimera blastomeres contributed to either extra-embryonic or fetal tissue, various PCR methodologies including nested PCR, AS-PCR, RT-PCR and real time PCR will be employed as described in 1.1.L. We further expect to find that NT-ES cell chimera will implant following embryo transfer and demonstrate axis development as imaged dynamically by high resolution U/S and MRI imaging. NT-ES cell-derived chimera rates may be slightly lower compared with the viability of non-manipulated controls or even ♀♂+♀♂ chimera. Therefore, we predict that we will need to produce 120 chimeras by aggregating ♀♂ or 4N embryos with NT-ES cells. Chimera development will demonstrate normal blastocyst formation as analyzed by total cell proliferation, intercellular interactions, compaction, and cavitation.

### EXAMPLE I

### 3. What is the Level of DNA Methylation in Embryos, Fetuses, Placentae, and Offspring After ART, NT, or Natural Coatings?

Rationale: Using an antibody to 5-methylcytidine, we first will address the global level of DNA methylation in fertilized NHP embryos after IVF or ICSI to determine if the paternal genome rapidly loses methylation after sperm incorporation, as reported in the mouse and bovine systems. Next, we will determine the DNA methylation patterns observed during preimplantation development to the expanded blastocyst stage in IVF and ICSI derived embryos using standard in vitro culturing techniques. These observations will be compared to in vivo fertilized blastocyst flushed from the uteri of Al or naturally fertilized females. We will also explore the global level of DNA methylation after somatic cell nuclear transfer and in vitro development to the expanded blastocyst stage. Finally, we will examine DNA modifications in early post-implantation development by exploring epiblast on Day 15-18 post-coitus and during germ cell development (D25-30 post-coitus).

3.1.A. Production of Fertilized NHP Embryos: Appropriately staged nhp embryos after 1VF or ICSI will be provided by Part V as detailed in section 1.1.G. Since the reported demethylation of the paternal genome in mouse and bovine zygotes happens within 4 hours of sperm incorporation, we will initially analyze 1VF and ICSI-derived oocytes at 2, 6, 12, and 24 hr post insemination. Baseline methylation of the paternal genome will be established by probing mature ejaculated sperm. We will also collect 2-to-4-cell, 8-to-16-cell, morula and expanded blastocyst stage for both IVF and ICSI-derived embryos for determining DNA methylation patterns.

3.1.B. Production of SCNT nhp Embryos: Appropriately staged SCNT nhp embryos will be produced as described in Part 1. We will analyze SCNT-derived embryos within 1 hr post NT as well as at 2, 6, 12, and 24 firs post-ionomycin/DMAP to contrast with IVF/ICSI derived embryos. As for ART-derived embryos, NT-nhp embryos will be harvested at the 2-to-4-cell, 8-to-16-cell, morula and expanded blastocyst stages to determine DNA methylation patterns.

3.1.C: Production of NHP Blastocyst from Natural Mating or Al is described in detail in Part V. Basically, superstimulated females will be place with a male stud following r-hCG administration for natural mating or inseminated artificially as described in Part V. Blastocyst will be flushed from the uteri of impregnated females 6 days post mating (day 0=the day following r-hCG) or Al using the techniques of (Seshagiri et al., 1993, Human Reprod 8:279-287) as described in the Part V. Recovered blastocyst will be collected in TALP-Hepes media and immediately processed for methylation detection without further in-vitro culturing.

3.1.D: Production of NHP post-implantation tissues. Isolation of early post-implantation embryos (epiblast cells; Day 14-18) and embryonic germ cells (day 25-30) will be performed after fetectomies as described in Part V, section 2.1.E, and Projects II and III, respectively. To determine methylation levels in newborn and adult somatic tissues, we will examine methylation patterns in fibroblast cells isolated from newborn offspring and in adults, as described in 2.1.B.

3.1.E: Detection of Global Methylation Patterns: After fixation in absolute methanol for 15 min or 4% paraformaldehyde (Polysciences) for 24 hrs. Fixed cells will be permeabilized with PBS containing 1% Triton X-100 for 40 rains and then treated with 3N HC1 for 20 min to denature the DNA. After washing in PBS, the fixed cells are blocked in PBS containing 3 mg/ml BSA and 150 mM glycine to reduce non-specific antibody binding. Global methylation will be determined using a mouse monoclonal antibody specific for 5-methylcytidine (5-MeCyd; Serotec, Oxford UK) followed by 488 Alexa-conjugated goat antimouse IgG secondary antibody to detect DNA methylation. After staining cells will be mounted on slides in Vectashield prior to imaging as described in Part IV. Epiblast will be manually dissected from surrounding tissues and embedded in Tissue-Tek OTC compound (Sakura) for preparing cyrosections (5-7 um) for immunohistochemical staining as described in Part II, Aim 1. Primordial germ cells (PGCs) will be isolated from excised Day 25-30 fetuses as described in Part V and fixed for immunocytochemistry as described in Part III, Aim 2.

3.1.F: Collection and Analysis of Data: We plan to do a minimum of 5 zygotes or embryos for each timepoint after IVF, ICSI, or SCNT and replicate at least 3 times. We will flush a minimum of 5 stimulated females to collect in vivo fertilized blastocysts for comparisons with ART and NT-derived blastocyst to determine if NHP embryos preferentially gain methylation in the ICM vs. the trophectoderm. We also anticipate performing 5 fetectomies each for epiblast and EG cell isolations: All fixed samples of zygotes, embryos or tissues will be examined by laser scanning confocal microscopy using a Leica TCS SP2 confocal microscope as described in Part IV. As the evaluation of fluorescence micrographs can be subjective, quantitative measurements of fluorescence intensity will be performed as previously described for 5-methylcytidine imaging in sheep embryos (Beaujean et al., 2004b, Biol Reprod 71:185-193). The Leica TCS SP2 microscope has settings to permit identical collection parameters for subsequent images. Individual images will be normalized against cytoplasmic background staining as this should be consistent from cell to cell. A region of interest will be drawn around each nucleus and the integrated fluorescence measured relative to the background staining.

Question 3.2. What are the parental expressions of selected imprinted genes during NHP preimplantation development? Rationale: This aim will fully utilize knowledge generated in Projects II and III regarding genomic imprinting in NHPs to solve clinically urgent concerns regarding ART epigenetic consequences. Specifically, we wish to explore if key genomic imprints identified in the mouse and human gametes are conserved in NHPs, i.e. Are the genes Igf2, Snrpn, Peg3, IPW, DlkI and KCNQ1OT1 expressed only from the paternal allele in NHP embryos? Are the genes H19, Gtl2, SLC22A18 NESPS5, KCNQ1 and Cdknlc expressed only from the maternal allele in NHP embryos? Performing these experiments in Part I establishes a baseline for comparison of the epigenetic parameters between human and nhp ESCs, as carried out in Part II, and with nhp EGCs as carried out in Part III. Our objective in this sub-aim is to determine the parent-specific expression of key imprinted genes that will be analyzed in Part II, focusing on post-implantation development of embryonic and extraembryonic lineages and in pluripotent stem cells and in Part III evaluating the erasure of imprints in embryonic germ cells. A core set of imprinted genes has been selected for analysis by all three projects. These genes were selected for analysis on the basis that they have been extensively studied in mouse embryos and ESCs, or have already been analyzed extensively in hESCs. An important feature of this set of imprinted genes (of >75 known in mouse and or human) is that they are globally, not tissue-specifically, expressed. Thus, their transcripts should be in all tissues studied, including embryos, undifferentiated pluripotent stem cells, and their differentiated progeny. The Igf2/H19 and Dlk1/Gtl2 gene pairs are selected because of their well-characterized DNA methylation regions (DMRs), and the tendency for H19 to undergo relaxation of paternal repression as a consequence of in vitro embryo culture (Mann et al., 2004, Development 131:3727-3735) as well as in cultured hESCs. These investigations poses intriguing questions as to the conservation of disregulations mechanism, asking whether it is common to both gene pairs in primates.

3.2.A. Production of Fertilized or SCNT NHP Embryos is described in 3.1.A and 3.1.B, as provided by Part V.

3.2.B. Experimental Design: The expression of imprinted genes in single Rhesus embryos will be accomplished using the single nucleotide primer extension (SNuPE) method (Szabo et al., 1995, Genes Dev 9:3097-3108), which has been used successfully in mouse embryos to measure allelic expression and is useful because of its sensitive and quantitative nature. With this approach we expect to be able to analyze the allele-specific expression of a single imprinted gene in each preimplantation embryo. The selection of genes for analysis in each mating will be determined by the parental polymorphisms, which will be analyzed by DNA sequencing of relevant coding regions (as determined by Part III) in advance of performing ICSI in order to maximize the number of embryos and therefore the number of informative imprinted genes for each cohort of embryos. SNuPE is highly sensitive and it is expected that we can measure expression from a single NHP embryo as has been shown previously for mouse embryos (Szabo et al., 1995, Genes Dev 9:3097-3108). In mouse methylation experiments differential expression isn't observed until after the transition to embryonic expression (MET) of genes as before this time expression will mimic maternal expression. We will examine two timepoints prior to the MET, two timepoints at the MET and two timepoints after the MET. Additionally we will isolate inner cell masses (ICM) from blastocysts by immunosurgery to measure differential expression between the ICM and trophectoderm. This sub-aim will require 385 embryos from Part V.

TABLE 2
DETERMINATION OF EMBRYO NUMBERS FOR AIM 3.2

| | | 4- | 8- | 16- | | | ICM after | | |
|---|---|---|---|---|---|---|---|---|---|
| | Zygote | cell | cell | cell | Morula | Blastocyst | Immunosurgery | Total | Σ |
| SNuPE | 5 | 5 | 5 | 5 | 5 | 5 | 5 | 35 | |
| | | | | | | | Numbers of | ×11 | 385 |
| | | | | | | | genes | | |
| | | | | | | | analyzed | | |

3.2.C. RNA Isolation, RT-PCR and SNuPE: The methods described for mouse embryos will be employed (Szabo et al., 1995, Genes Dev 9:3097-3108). Embryos are mechanically stripped of all somatic cells (primarily cumulus cells) in the presence of 2 mg/ml hyaluronidase and washed extensively in TALP-Hepes. The zona pellucida is removed using a brief incubation in 5 mg/ml Pronase (Sigma) and the embryo washed. RNA is isolated from single embryos by placing individual embryos into 10 μl aliquots of RNAzo1 followed by snap freezing and storage in LN2 until assayed. A cDNA is generated by preparing a reaction containing: 5 mM MgC12, 1×RT buffer, 1 mM each dNTP, lunit/μl ribonuclease inhibitor, 15 unit/μg AMV RT, 0.5 μg random hexamers, and nuclease free water to a 20μ1 final volume. This reaction is added to the RNA and incubated at room temperature for 10 min and then 42° C. for 15 min. Finally the sample is heated to 95° C. for 5 minutes, and immediately placed at 4° C. for PCR. PCR primers are designed that span polymorphisms identified in the rhesus colony at the Pittsburgh Development Center. SNuPE is performed in a reaction mix containing 10 ng of the PCR product, 1 μM of the SNuPE primer 10 mM Tris-HC1 (pH 8.3), 50 mM KCl, 2 mM MgC12, 0.75 units of AmpliTaq DNA polymerase, and 2 μCi of [32P]dNTP. One cycle is run; 95° C. for 30 sec, 42° C. for 30 sec and 72° C. for 1 min. Bands are separated by electrophoresis and imaged using a phosphor imager.

3.2.D: Interpretation and potential pitfalls: The selection of imprinted genes for analysis in preimplantation Rhesus embryos will depend on the availability of single nucleotide polymorphisms in coding regions of imprinted genes in the breeding stock of Rhesus monkeys allocated to this project. However, there is a substantial level of polymorphism in the Pittsburgh Rhesus colony, as indicated by the preliminary sequencing data obtained in Part III. Thus, while we cannot predict with certainty that each of the imprinted genes targeted for analysis will be successfully studied, it is likely that the majority will be amenable to study. The SNuPE method is highly sensitive and specific, owing to the use of the polymorphic nucleotide as the labeled moiety for the primer extension reaction. Nevertheless, the technique demands high attention to protect the reaction mixture from contamination from other sources, and to preserve the mRNA in the sample from degradation. These objectives can be ensured by the practice of setting up RNA amplication reactions with the protection of a laminar flow hood, aerosol-free tips, and in the continuous presence of a cocktail of RNAse inhibitors. Additionally, preferential amplification of one allele compared to the other allele by PCR bias could alter the interpretation of the results. We will circumvent this problem by assaying known ratios of each allele from parental stock and identifying any biases. PCR bias has not been observed in our expression studies. We will determine the sensitivity of the reaction by limiting dilution. If SNuPEis not successful we can also employ Molecular Beacons and real-time RT-PCR to quantify expression from each allele (Mhlanga et al., 2001, Methods 25:463-471; Sevall, 2001, Methods 25:452-455).

Question 3.3. What is the DNA methylation status of specific genes in Rhesus preimplantation embryos? Are the imprint control regions for selected genes methylated on the paternal or maternal genomes in Rhesus embryos as they are in human embryos? Rationale: To further measure the genomic imprinting in NHPs, we will determine if the specific differentially methylated regions (DMR), known to represent the gametic imprints for either maternally or paternally expressed genes in other species, are also methylated in non-human primates. This information will provide the baseline for understanding and interpreting imprinting analyses in Part III, involving expression in primary germ layers and their derivatives, in ESCs, and in EGCs. For this purpose, we will consider methylation studies of four regions, two that are methylated on the paternal allele, regulating H19/Igf2 and DlkI/Gt12 imprinted gene pairs on human chromosomes 11 and 14, respectively; and two that are methylated on the maternal allele, KCNQ1 (Beckwith-Wiedemann syndrome-related) and Snrpn (Angelman syndrome related) on human chromosomes 11 and 15, respectively. The targets for initial analysis are H19/Igf2 and KCNQ1, based on the extensive information about their imprinted expression and methylation status in the mouse. The objective of these experiments will be to assess the methylation status in preimplantation stage Rhesus embryos, specifically at the blastocyst stage. The selection of this stage for analysis reflects the minimum requirement of ˜100 cells for the DNA methylation analysis by the bisulfite method, which involves conversion of unmethylated cytosines to uracils, which are detected as thymines upon DNA sequencing. This method has been recently refined so that it is possible to assess methylation in single preimplantation mouse embryos (8 cell stage or later), and this is the approach that will be applied for this purpose (Millar et al., 2002, Methods 27:108-113; Warnecke et al., 1998, Genomics 51:182-190). 3.3.A. Production of Fertilized nhp Embryos: is described in 3.I.A and the Part V.

3.3.B. Experimental Design. The methods for bisulfite sequencing have been refined so that they can reliable amplify DNA from as little as 5 diploid genomes. At these very low concentrations the technique suffers from stochastic amplification of alleles. To avoid this possibility we will isolate no fewer than 20 diploid genomes for each timepoint and to avoid an individual embryo bias all timepoints will include at least two embryos and all experiments will be repeated in triplicate. In this way we can have reasonable confidence in the results.

TABLE 3

| | Zygote | 4-cell | 8-cell | 16-cell | Morula | Blastocyst | Total | Σ |
|---|---|---|---|---|---|---|---|---|
| Bisulfite | 20 | 5 | 5 | 5 | 5 | 5 | 45 | |
| Sequencing | | | | | | Number of regions | ×4 | |
| | | | | | | analyzed | | |
| | | | | | | Number of | ×3 | 540 |
| | | | | | | Repetitions | | |

3.3.C. Isolation of DNA from Embryos. DNA will be isolated from embryos essentially as described previously for mouse embryos (Millar et al., 2002, Methods 27:108-113; Warnecke et al., 1998, Genomics 51:182-190). Embryos in the quantities identified above will be mechanically stripped of any adhering maternal cells and concentrated in 2-5μ1 of PBS. Embryos are then resuspended in 18 μl of the following solution (2 μg Escherichia coli tRNA, 1 mM SDS and 280 μg/ml proteinase K). This solution is incubated for 30-90 minutes at 37° C. followed by incubation for 15 min at 98° C. under mineral oil.

3.3.D. Bisulfite Treatment of Isolated DNA: The samples described above are denatured by addition of 2 μl of 3M NaOH for 15 min at 37° C. The DNA is bisulfite treated by the addition of 208 μl 2.3 M sodium metabisulfite, pH 5.0 resulting in a final concentration of 2.0 M, 12 μl 10 mM hydroquinone, final concentration 0.5 mM and 2 μg E. coli tRNA. This is incubated at 50° C. for 4-16 hrs. The sample is desalted using a Wizard DNA Clean-up Kit (Promega) and the eluted DNA is desulfonated using 3 M NaOH for 15 min at 37° C. The DNA is precipitated with ammonium acetate (pH 7.0), centrifuged, and resuspended in 20 μl of water and stored at −20° C.

3.3.E. PCR of Bisulfite-Treated DNA: We will choose primers that span differentially methylated regions and which contain informative polymorphisms. Nested PCR will be used to amplify low quantities of DNA. PCR products will be subcloned into plasmids, transfected into chemically competent E. coli and selectively grown on agar plates containing antibiotic. Approximately 20 colonies from each PCR reaction will be chosen for sequencing using a BigDye Terminator Cycle Sequencing kit in conjunction with an automated capillary sequencing system (Applied Biosystems; Foster City, Calif.). Resulting sequences will be compared against one another to obtain a ratio of differentially methylated regions present within the embryo.

3.3.F. Interpretation and Potential Pitfalls We could potentially encounter PCR Bias, stochastic PCR amplification and cloning bias of the PCR fragments prior to sequencing. PCR bias (one allele being preferentially amplified over the other) can be controlled by first testing the primers on DNA with known ratios of fully methylated and unmethylated DNA. Only primers that demonstrate no bias will be employed. It is possible when amplifying small amounts of DNA that amplification results from only one initial DNA molecule (stochastic PCR amplification). This can also be tested by limiting dilution of known ratios of DNA and using no less DNA than that which gives amplification of both alleles (Warnecke et al., 1998, Genomics 51:182-190). Additionally when working with small amounts of DNA it is possible to lose the DNA in the reaction. The method above uses carrier DNA to control for this and has been successfully employed in mouse embryos. An additional technique is to isolate the DNA in low melting temperature agarose and process the agarose block further. This technique that will be used is sufficiently sensitive for the small numbers of rhesus embryos that will be available. Finally there is potential for cloning bias, the preferential v cloning of one PCR fragment over another in E. Coli. This can again be controlled by transforming cells with a known ratio of DNA and measuring the outcome after cloning and sequencing.

### EXAMPLE 1

### 4. Dynamic Imaging of ES Cell Fates After Transplantation

Rationale: In this Aim, we explore differentiated stem cell fates after allograft transplantation (4.2) and immune matching of NT-nhp-ESCs in the first question. We ask if pluripotent NT-nhp-ES cells transplanted into the kidney capsule of the autologous female (i.e. cloned stem cells back into the NHP that provided both the egg and the somatic cell) are indeed immune matched and tolerated as a prelude to designing nonhuman primates disease models which might be cured through stem cell technology following transplantation. Next, we test whether nhp-ESC and NT-nhp-ES cells stably differentiated into neural or hematopoietic stem cells lineages, as well as from HESC, are immunotolerated when transplanted into localized sites in NHPs (e.g. subcutaneous, testicular, intramuscular or kidney capsule). The objective is to determine the long-term stability and fates of differentiated ES and NT-ES cells in vivo. Table II (Introduction) provides the overall experimental strategy.

Question 4.1. Are autologous NT nhp-ESC transplantations into localized regions (e.g. kidney capsule, subcutaneous, intramuscular) immune matched and tolerated? These autologous NT-rihp-ESCs are generated in three manners:
Female Autograft: SCNT in which the cumulus donor cell and oocytes are from the transplanted female;
Fetal Fibroblast Autograft: Amniotic cell NT transplanted to born NHP;
Male and/or Female Autograft: Somatic fibroblast NT into unrelated oocyte, transplant to nuclear donor.

4.1.A. Derivation of pluripotent NT-nhpES cell is described in 1.1.D, 2.1.H and Part VI.

4.1.B. GFP transgene insertion into NT-nhpES cells is described for nhpES cells in 1.1.G.

4.1.C. SPIO Labeling of pluripotent NT-nhpES cells for in vivo MRI Imaging is described in Part IV. Two labeling strategies, each successful (cationic lipids and receptor-mediated endocytosis), are described in detail in Part IV. SPIO particles obtained commercially (Miltenyi Biotec Inc., Auburn, Calif.). For transfection, SPIO particles are pre-mixed with the transfection agent Lipofectamine 2000 for ˜20 minutes before adding to the culture media. Previously, we determined that 6 μl/ml of Lipofectamine provides effective coverage of the SPIO particles (see Part IV). Differentiated HESCs, nhp-ESC and NT-ES cells (˜10⁵-10⁷ cells) will be harvested (Project VI) labeled while in suspension at 37° C. in a humidified 5% CO₂ atmosphere. At the end of the incubation period, the ES cells will be washed twice to remove excess agents prior to transplantation as described next.

4.1.D. NHP Stem cell transplantation: Pluripotent nhpES cells are harvested by cell scraping followed by treatment in 0.25% trypsin-EDTA for 5 minutes to break up cell colonies. Individual cells along with small colonies will be washed twice in sterile PBS and the final concentration between 0.05-5×10⁶ in 0.4 ml. NHPs are anesthetized (Project V) and the entire volume of the stem cells transferred subcutaneously, intramuscularly, or to the kidney or testicular capsules, as described in Part V.

4.1.E. Stem cell transplantation into SC1D mice will be performed as described in 1.1.F.

4.1.F. MRI Imaging is performed as described in the Part IV. Image frequency will be once every two weeks for the first two months and thereafter on a monthly basis providing no rejection is observed.

4.1.G. Analysis of teratomas or biopsied material. Teratoma formation, as determined both by palpation and imaging, will result in tumor excision (as in Part V) and subsequent fixation in 4% paraformaldehyde (EM grade; Polysciences). After embedding in paraffin, sections will be processed for histology. Typically, 8-μm sections are cut and stained with hematoxylin for identification of tissue types (endoderm, ectoderm and mesoderm). Some sections will be labeled with specific tissue markers (Tuj1:neuron-specific beta-III tubulin; MF20: anti-myosin, mesoderm; alpha-fetoprotein: endoderm marker as in Imaging Part IV. Images of sections are collected by conventional or confocal microscopy.

4.1.H. Mt-DNA and RT-PCR analysis on recovered tissues or teratomas will be performed as described in 1.1.L.

4.1.I. Data collection and analysis. We anticipate analyzing at least three individual NT-nhpES cell lines derived from separate blastocysts, each fully characterized, maintained and cultures by Stem Cell Part VI. Nine females (3 for each cell line) will initially be analyzed for immunotolerance. At each injection site, we will monitor for evidence of inflammation versus cellular proliferation, differentiation, or possible migration over 5-8 weeks (see Part V for more details). The kidney capsule is recommended because it is immunoprotected, easy for NT-nhpES cell transplantation, and can be monitored by MRI imaging. Evidence of teratoma formation at subcutaneous, testicular, intramuscular sites are determined both by palpation and MRI imaging analysis as described in the Imaging Part IV. We will retrieve biopsied material from suspected teratomas identified in nonhuman primates without sacrificing animals. For analysis of collected tissues, we will use static immunocytochemistry, using specific antibodies to neuronal or hematopoietic lineages, to identify tissue differentiation and EM sectioning of tissues to determine cellular organization. Also, we will use RT-PCR to detect GFP transgene and mtDNA analysis to confirm that the recovered tissue is derived from the transplanted differentiated stem cells. The numbers of cells for transplantations has been determined in pilot MRI experiments, as demonstrated in Part IV. We anticipate injecting between 0.5-5×10⁶ NTnhpES cells into recipient animals to explore immunotolerance. These numbers are based on preliminary data obtained in transplantation of SCID mice with human and mouse ES cells after SPIO labeling for MRI analysis (Project IV).

4.1.J. Expected results. We expect that NT-nhpES cells are immunotolerated when transplanted to the female from which the very NT blastocyst was initially derived (i.e. both donor nucleus and oocyte). We expect that the NT-nhpES cells will form teratomas demonstrating all three germ layers (endoderm, ectoderm and mesoderm) as confirmed by specific marker antibodies (see 4.1.G) for each tissue type and high resolution EM analysis of tissue sections. We will confirm that the collected teratoma was derived from the NT-nhpES cells by analyzing mtDNA and detecting the inserted GFP transgene as described in 1.1.L.

Question 4.2 Are allogenic ES cells differentiated into neural or hematopoietic stem cell lines rejected like allografts or do their pre-implantation origins confer immunological privilege? Strategy: This question asks if differentiated nhpES or NT-nhpES cells transplanted into random females will be tolerated after transplantation in vivo.

4.2.A. Derivation and GFP transgene insertion of pluripotent nhpES and NT-nhpES cells as in 4.1.A-B.

4.2.B. Differentiation of ES or NT-ES cells. We will prepare two differentiated stem cells populations for transplantation:

4.2.B.i Neuronal Differentiation of ES or NT-ES cells: Pluripotent ES stem cells are cultured under conditions that promoted differentiation into neural progenitor (NP) cells. Two protocols were employed that produced 80-90% NP cells, one, an adherent protocol that required a graded reduction of mouse feeder cells and the second, a feeder free protocol that required a specific period of culture as embryoid bodies_ NP cells cultured by these methods retained markers of progenitor cells for more than 2 months. These cultures could be further driven to neuronal lineages by media conditioned by glial cells. We will use antibodies to specific markers of neuronal cell lineages (beta-tubulin III, nestin, NCAM; Mushashi; Sox-1) to confirm differentiation and to estimate the purity of our population prior to transplantation studies. The static inununocytochemistry technique has been described in Part VI.

4.2.B.ii. Hematopoietic stem cell. Successfully gene-targeted NHP ES cell lines will be differentiated to hematopoietic cells through adaptations of our novel human EB methods, or via co-culture on murine bone marrow stromal lines as described by Honig's group (Banuelos et al., 2004, Clin Immunol). CD34+-sorted control and gene-targeted NHP EB cells will be re-cultured on OP9 bone marrow stromal layers, or NOD-/scid fetal thymic organ cultures (FTOC)(Robin et al., 1999, Br J Haematol 104: 809-19) in the presence of Kit-Ligand, Flt-3 Ligand, Thrombopoietin, IL-2, IL-15, and IL-7 for the generation of B and NK (OP9 system) (McCune, 1991, Curr Opin Immunol 3:224-28; Vugmeyster et al., 1998, Proc Nat'l Acad Sci 95: 12492-97) or T lymphoid cells (FTOC). We will analyze by FACS differentiated NHP ES cells for rhesus CD19, IgM (B-lineage) and CD3/CD4/CD8 (T-lineage).

4.2.C. Labeling Differentiated ESCs for in vivo MRI Imaging is described in 4.1.C.

4.2.D. NHP Stem cell transplantation: Differentiated hematopoetic SC, nhpESCs and/or NT-nhpESCs are analyzed as in 4.1.D and Part V.

4.2.E. Stem cell transplantation into SCID mice will be performed as described in 4.1.E, 1.1.F and Part VI.

4.2.F. MRI Imaging is performed as described in the Imaging Part IV. Image frequency will be once every two weeks for the first two months and thereafter on a monthly basis providing no rejection is observed.

4.2.G. Analysis of teratomas or biopsied material will be described as in 4.1.G.

4.2.H. Mt-DNA and RT-PCR Analysis on recovered tissues or teratomas will be performed as described in 1.1.L.

4.2.I. Data collection and analysis: is similar to 4.1.I. We anticipate that we will test 3 lines of each cell type in at least three animals.

4.2.J. Expected results: We expect to show that specific markers neuronal or hematopoietic cell types will demonstrate successful differentiation in vitro with >95% purity in the population. We anticipate that transplantation of differentiated nhpES or NT nhpES cell lines into random, non-matched adult rhesus monkeys will not be immunotolerated, resulting in no teratoma formation at the injection site and few, if any, migratory cells as imaged by MRI. Inflammation of the injection sites will be closely monitored by the attending veterinarian and experiments terminated if these animals demonstrate discomfort (see Part V for discussion). Conversely, we expect to demonstrate single lineage tissue formation in SCID mice that will proliferate neuronal or hematopoietic cells in these immunocompromised mice, and we will confirm this after sacrifice and analyzing the excised tissues.

### Part II

### Differentiation and Epigenesis of Pluoripotent Stem Cells

In order to reliably direct stem cell differentiation into tissues useful for transplantation therapies, it will be necessary to establish definitive molecular markers for differentiative pathway(s) of interest. This objective is complicated by the complete unavailability of the requisite early postimplantation stages of human conceptuses, when early tissue allocation and differentiation are initiated. It is therefore proposed to compare the stage and tissue-specific transcriptional profiles of non-human primate and mouse embryonic tissues using DNA microarrays to establish the molecular identities of pluripotent stem cells and their early differentiated progeny in vitro and in utero. See Examples II 1 and 4. The bioinformatics strategy is designed to identify a set of robust molecular markers that characterizes each stage and tissue lineage of the early mammalian conceptus. This analysis provides the basis for inducing stem cell development along specific tissue lineage pathways, and it establishes benchmarks for judging the normalcy and stability of differentiated states achieved by in vitro differentiation.

It is also important to define the epigenetic status of pluripotent stem cells and their differentiated progeny. This will be accomplished through an analysis of the allele-specific transcriptional activity of imprinted genes in pluripotent stem cells and in non-human primate embryonic tissues using single nucleotide polymorphisms. See Examples II 2 and 5.

These studies will be extended through an experimental approach involving perturbation of the epigenetic state of pluripotent stem cells, in order to determine the mechanisms responsible for maintaining epigenetic stability in such cells. The developmental potential of pluripotent non-human primate stem cells having either normal or experimentally perturbed expression of imprinted genes will be assessed by generating chimeras and evaluating their developmental normality. See Examples II 3 and 6. These results will lay conceptual foundations for the eventual therapeutic use of pluripotent human stem cells and their differentiated progeny, and also for addressing concerns regarding the influence of assisted reproductive techniques (ART) on epigenetic status of the early conceptus.

### EXAMPLE II

### 1. Transcriptional Profiling of Tissue Lineages Derived from Mouse and Human ES Cells and Early Embryos

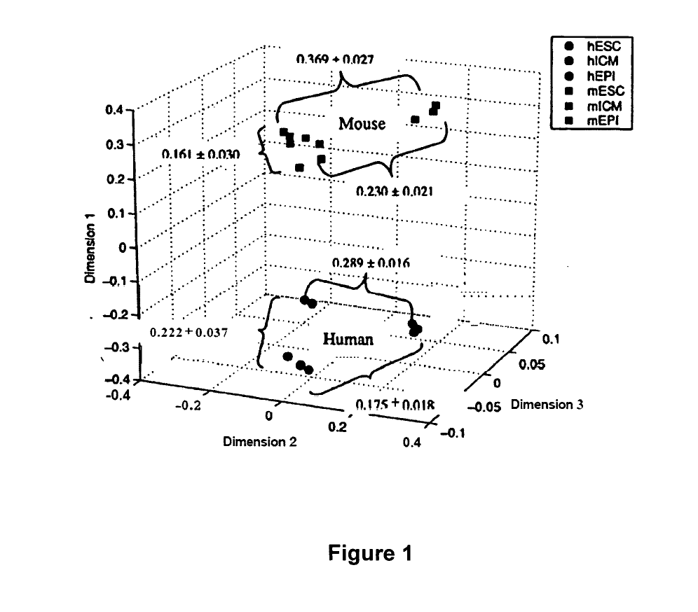

The mechanisms of ES cell pluripotency and its maintenance are important issues with both fundamental and practical implications. In particular, directing hESC differentiation requires knowing what developmental stage they represent. Cultured mES cells spontaneously differentiate to cells with characteristics of ectoderm, mesoderm or endoderm, suggesting that they reflect the potentiality of the epiblast, rather than the ICM (inner cell mass). Surprisingly, however, hES cells can differentiate to trophectoderm cells (Xu et al., 2002, Nat Biotechnol 20:1261-1264), unlike mouse ES cells, suggesting that they could have the potentiality of the earlier, morula stage. Therefore, we undertook to identify the embryonic stage(s) that rnES cells and hES cells represent, as this may be critical in understanding the mechanisms that maintain their pluripotency and what signaling pathways control their differentiation. The transcriptional profiles of ES cells and ICM in mouse and human tissues were compared using the analysis of Affymetrix® microarrays (Affymetrix, Inc., Santa Clara, Calif.) to determine whether the developmental state of ES cells resembled more that of their respective ICMs, or their epiblast. The amplification method of (Tietjen et al., 2003, Neuron 38:161-175) was used with RNA obtained from single hESC colonies of the H9 hES cell line and from single mouse (L. Smithers, Free University of Belgium, Brussels) or human inner cell masses (H. Van der Velde and A. Van Steirteghem, Free University of Belgium, Brussels). These were compared with epiblast-like cell layers microdissected from Nodal-expressing ernbryoid bodies. Transcriptional profiling was carried out similarly using RNAs isolated from single colonies of mES cells, mouse ICMs, and E6.5 and 7.5 epiblast. The results suggest that mouse ICMs are transcriptionally more similar to their respective epiblast that to their ESCs (FIG. 1). This extends the conclusion of (Sharov et al., 2003, PLoS 1:E74), who found rnES cells were more similar to epiblast than to whole blastocysts, on the basis of EST frequencies. These results from both these highly amplified transcriptional profiles and from standard Affymetrix® profiling provide the identities of genes that are responsible for the unique expression patterns of pluripotent cells, as contrasted with their differentiated progeny. These gene lists confirm and extend recently published observations (reviewed by (Rao, 2004, Dev Biol 275:269-286)), and provide the basis for selecting candidate “pluripotency” genes for further intensive analysis.

In this preliminary study, human and mouse arrays were compared that were background corrected, normalized and summarized using default parameters of the RMA model (Irizarry et al., 2003, Nucleic Acids Res 31:e15). All array processing was performed using the ‘affy’ package of the Bioconductor suite of software (Fred Hutchinson Cancer Research Center, Seattle Wash.) or the R statistical programming language (The R Foundation for Statistical Computing, Institut für Statistik und Wahrscheinlichkeitstheorie Technische, Universitat Wien, Vienna, Austria). The resulting human and mouse gene expression data sets contained processed expression values for 54,675 transcripts (Affymetrix® hg-u133+2 chip, Human genome) and 12,488 transcripts (Affymetrix® mg-u74Av2 chip, Murine Genome U74v2 Set) respectively. The expression sets were analyzed to assess the significance of differential gene expression between ESC and ICM groups (3 arrays in each). The moderated t-statistic of Smyth (2004, Statistical Applications in Genetics and Molecular Biology 3(1) Article 3) was applied to both human and mouse expression sets to assess the significance of differential expression between ESC and ICM groups for each transcript present. In order to reduce the errors associated with multiple hypothesis testing on such a scale, the significance p-values obtained were converted to corrected q-values using the method of (Storey and Tibshirani, 2003, Proc Natl Acad Sci USA 100:9440-9445). Transcripts with associated q<0.01 were deemed to exhibit significant differential expression between ESC and ICM groups. A total of 3938 human genes and 500 mouse genes were labeled significant by this threshold. Of the 3938 human gene transcripts deemed to display significant differential expression, 1752 were identified as having orthologous probe-sets on the mouse chip, using a list of probe-set orthologs obtained from Affymetrix® (multiple probe-set associations were preserved). Of these 1752 transcripts, 127 possessed (one or more) orthologs that were labeled significant on the mouse expression data as well. The final list includes 132 orthologous transcript pairs that display significant differential regulation between ESC and ICM groups on both mouse and human data. The orthologous pairs include 127 human transcripts and 98 mouse transcripts (some appearing more than once, as multiple probe-set associations were preserved when assigning mouse orthologs to human gene probe-sets). An interesting initial observation is that 28 of the 132 pairs display contrasting differential regulation (e.g. a transcript up-regulated in human ESC, with ortholog down-regulated in mouse ESC).

These results show the value of transcriptional profiling as a means of comparing the identities of pluripotent cells in vivo and in vitro. These comparisons also reveal the shortcomings of current comparisons within the human system, namely that the key pluripotent stem cell needed for comparison, human epiblast, is not available from bona fide human embryonic tissues. Therefore, it is not currently possible to assess the significance of the apparent similarity between human ICM and the hEPI-like cells derived from Nodal-expressing embryoid bodies. This gap compels us to turn to primate embryos a) as a relevant model to complement the mouse system as a means of defining the embryonic stage that hESCs most resemble, and b) for establishing transcriptional marker genes as benchmarks for characterizing in vitro hESC differentiation, again, in comparison with comparable mouse tissues.

FIG. 1 was generated by normalizing human and mouse microarray data using 6161 uniquely orthologous probe-sets from Affymetrix hg-u133+2 (human) and mg-u74Av2 (mouse) GeneChip microarrays. The plot in FIG. 1 was created using multidimensional scaling (Kruskjal, 1964, Psychometrika 29:1-27) on the 6161 gene expression scores used to describe each sample. The inter-group distances shown in FIG. 1 were calculated from the untransformed expression profiles using the inter-group distance analysis method of method of (Wang et al., 2004, Nat Genet 36:687-693).

### EXAMPLE II

### 2. Assessment of the Epigenetic Status of hESCs and Their Differentiated Progeny

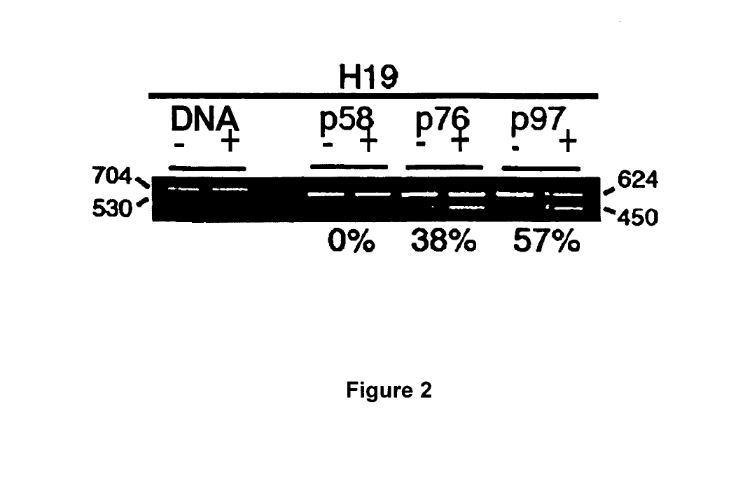

The evaluation of the epigenetic status of hES cells has focused on establishing a robust approach for analysis of genomic imprinting in this system. Informative polymorphisms in four hES cell lines (H9, H7, hSF-6 and HES-3) for allele-specific expression of six imprinted genes [IGF2, IPW, KCNQ1OT1 (paternally expressed set); and H19, SLC22A18 and NESP55 (maternally expressed set)] have been identified. There is generally monoallelic expression of imprinted genes in the hES cell lines studied to date (Vallier et al., 2004, Stem Cells 22:2-11). The expression of IGF2, IPW and KCNQ1OT1 was exclusively monoallelic in all lines and at all passages studied (p49-155). Similarly, expression of H19 was monoallelic at all passages (p58-155) in two of three lines studied and up to ˜p70 in a subline of H9. (This subline was obtained from Dr. Jamie Thomson at the University of Wisconsin in 1999, when Dr. Pedersen was at UCSF, and it was transferred to the University of Cambridge in 2001.) At higher passages (≧p76), biallelic expression of H19 was observed in this H9 subline (FIG. 2). This biallelic status appeared to be progressive with passaging, as the fraction of transcripts derived from the previously silent allele increased significantly with passage number (FIG. 2; P=0.02, t-test). Expression of SLC22A18 was predominantly from one allele (23-25% of total transcripts were from the ‘silent’ allele), and a similar pattern was seen for NESP55 (0-20% of total transcripts were from the ‘silent’ allele), although in neither of these latter two genes did the transcription of the ‘silent’ allele change significantly with passage number.

The epigenetic status of hES cells is relatively stable at low and medium passages (<p50), but that it may be susceptible to perturbation at higher passage in the case of specific gene(s) and cell lines. Intriguingly, the imprinted genes that showed detectable expression of the ‘silent’ allele (whether stable or progressive) were those in which the paternal allele is typically silent (i.e., maternally expressed imprinted genes H19, SLC22A18 and NESP55). The concurrence of derepression of paternally silent genes in human hES cells observed here and mouse embryos mutant for Eed (Mager et al., 2003) shows that hES cells provide a system for analyzing this intriguing phenomenon. Finally, while the anonymous condition of existing hES cell lines precludes a definitive assessment of parental allele origins, the consistency of the asymmetric regulatory pattern between the two parental alleles suggests that the ‘silent’ alleles are indeed derived from the expected parent of origin, and have not undergone a ‘switch’ in identity during hES cell derivation and culture processes. Finally, these results provide the basis for further analysis of hESC epigenetic status at mechanistic levels.

### EXAMPLE II

### 3. Methylation Status of Key Imprinting Control Regions

Bisulfite sequencing studies will be performed to evaluate the methylation status of key imprinting control regions. The bisulfite treatment protocol has now been optimized so that we are obtaining high (>95%) cytosine conversion rates, but still retaining one or two unconverted non-CpG associated cytosines to aid with the identification of unique clones. Control studies have been carried out as detailed in the experimental procedures in order to preclude any PCR or cloning bias in these assays. Primers and conditions for analysis of the H19 DMR have been identified and selected for the absence of any bias.

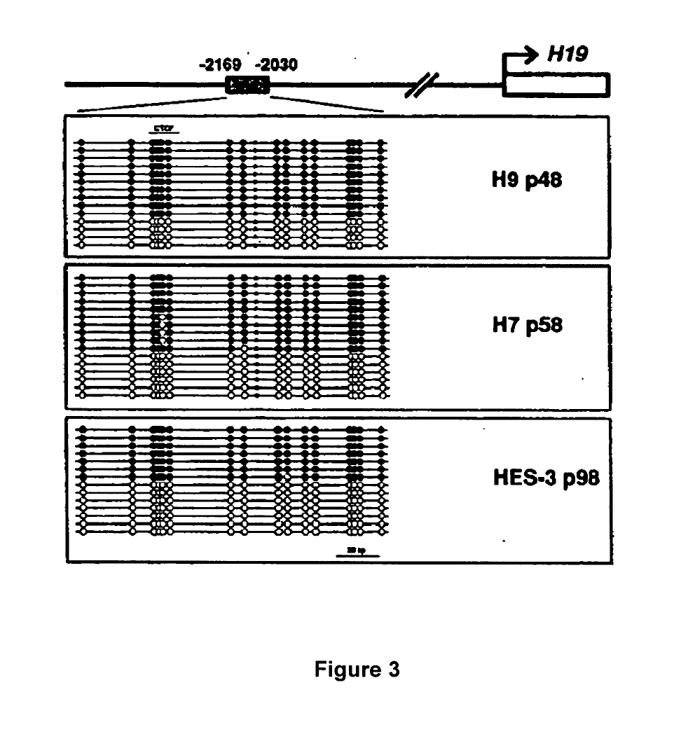

Preliminary data collected in three hESC lines (H9, H7 and HES-3) reveals that allele-specific methylation is retained in the H19 DMR (FIG. 3; note especially data for H9 p48). These findings are consistent with the maintenance of a stable epigenotype at early passages and furthermore concur with the preliminary data on predominantly monoallelic H19 imprinted gene expression in these samples. The presence of a SNP within the H19 DMR region of H9 and H7 hESC lines adds confidence of the data by allowing the DMRs of the two parental alleles to be distinguished.

Higher passage cells will be analyzed to see if there are significant methylation changes over prolonged passage. In addition, primers and treatment conditions will be optimized for the two other key imprinting control regions, KvDMR and SNRPN DMR.

### EXAMPLE II

### 4. Determine the Transcriptional Identity of Early Embryonic Cells and Their in utero Progeny

The pluripotency of embryonic stem cells commends their use in derivation of differentiated progeny with clinical potential. However, the genuine process of differentiation takes place in mammals in an intrauterine environment, aided by a three-dimensional structure and numerous factors that are acquired from the intraembryonic microenvironment during the course of normal development. The hypothesis that underlies the proposed use of pluripotent stem cells as exogenous sources of cells for transplantation therapies is that such cells and composite tissues will be comparable in function and stability of their differentiated state to in vivo generated tissues. While it can be argued that the successful development of mouse ESCs in chimeric circumstances proves the normality of ESC-derived tissues, the numerous differences that are emerging between mouse and primate ESCs renders this case less than compelling.

In thus attempting to validate the normality of cells and tissues generated from hESCs, we face the experimental limitations of the human species as an experimental system, owing to both technical and ethical limitations. In the first instance, the in vitro differentiation of hESCs cannot by itself be used as to establish criteria for the normality of in vitro derived tissues, at this would be self-referential. This highlights the need for establishing in vivo-derived benchmarks to validate the differentiated status of the early lineages generated by differentiation of pluripotent mammalian cells. A second issue is whether the pluripotent hESCs generated by cultivating human blastocysts actually represent the inner cell mass or rather a later stage, in terms of their comparability to normal development. This issue will of course have a significant impact on the rational design of differentiation regimes: if such regimes are to be based on knowledge of the embryonic milieu, which is known to change progressively with time, then hESCs themselves need to be staged in comparison with each of the several pluripotent cell types present in the early mammalian embryo (i.e., morula, inner cell mass, primitive ectoderm, epiblast and germ cells). Finally, there is a practical advantage to determining whether particular imprinted genes are transcribed or not (without regard to their allele specific expression, latter task being the objective of Aim 2) in early primate embryonic tissues lineages. Our hypothesis is that these issues can be resolved by a systematic comparison of human ESCs and their in vitro differentiated derivatives with pluripotent and differentiated cells of the non-human primate and mouse embryo.

Definition of markers: The first priority will be to validate a set of markers for the range of phenotypes expected for in vitro differentiation of hESCs. Robust markers of inner cell mass and epiblast will be identified, as well as definitive endoderm, ectoderm and mesoderm, plus extraembryonic mesoderm, amniotic ectoderm and visceral (extraembryonic) endoderm. There are several principal criteria for markers that define them as robust: they need to be unambiguously associated with the bona fide, native tissue as it develops in vivo, consistently expressed by genetically different individuals within a species, expressed across a broad time range (i.e., a day or more), rather than transiently. Expression will be detected in the first instance by transcriptional profiling using DNA microarrays. Candidate markers will then be further analyzed by a set of progressively more quantitative and qualitative methods, including RT-PCR to detect expression in cell populations, real time PCR, to assess absolute levels of mRNA, and in situ hybridization to determine the cellular distribution of transcripts.

Marker genes can be defined as those genes whose expression is correlated with an observed biological phenotype or difference between discrete conditions. Microarray experiments involve the capture of thousands of gene expression measurements for each experimental condition/tissue analyzed. The result is thousands of expression pairs, the difference between which may, in principle, be used to identify genes responsible for the biological phenomenon, or difference between conditions, described by comparison of any two array experiments. Many methods exist with which to identify the genes responsible for the differences in comparisons between two microarray experiments. Linear fold-change thresholds have recently been superseded by variable fold-change thresholds that incorporate the knowledge that genes have differing propensities to change according to their position on the recorded range of expression intensity.

The method proposed in this work (Trotter, M. W. B., Milian, E. J., Pedersen, R. A. and Buxton, B. F., (2004) Technical Report RN/04/17; Dept. of Computer Science, University College London, UK) provides a marker selection approach that incorporates and accommodates intensity fluctuations over the entire range of recorded expression. Differential expression ranked by the transformation method is more robust to changes in microarray data processing methods (such as normalization, and background correction) than when ranked either by a conventional z-score or by the sliding z-score of (Quackenbush, 2002, Nat Genet 32 Suppl 496-501). Moreover, the transform facilitates the application of sophisticated machine learning techniques, such as the data description method of Tax and Duin (2004, Machine Learning 54(1):3097-3108). Using this approach, it was relatively straightforward to detect marker gene expression characteristic for hESCs by comparison with their differentiated derivatives (see Trotter, et al. (2004)). This approach readily detected standard, known markers of pluripotency (Oct4, Nanog, FGF4, Nodal, Cripto and Lefty), among numerous others whose role has yet to be defined. Our objective in this aim is to define 10 or more robust marker genes for each of the tissues to be analyzed.

ii. Identification of transcriptional marker genes in non-human primate embryonic lineages: Transcriptional profiling of non-human primate embryonic and extraembryonic tissue lineages will be carried out to generate benchmarks for evaluating the differentiation of hESCs and nhpESCs along such lineage pathways. Because of the small amounts of tissues available for analysis of such lineages of early primate embryos, the analyses will be conducted using the amplification methods previously developed by our collaborator, Dr. Koentges and his colleagues (Tietjen et al., 2003, Neuron 38:161-175), and as previously used by us to compare the transcriptional profiles of mouse and human pluripotent cell types (see Examples II 1 and 2 and below for detailed methods). The primate embryos will be generated by Core B. Our first objective will be to generate transcriptional profiles for the inner cell mass, epiblast, and the primary germ layers and their earliest derivatives (definitive mesoderm, endoderm and ectoderm, as well as lateral plate mesoderm, gut and neuroectoderm) by laser capture microdissection of day 15-16 post-fertilization embryos. Such early stage embryos are exceptionally rare in humans, but can be straightforwardly obtained in the Rhesus monkey (Enders, 2002, Plancenta 23:236-238; Enders et al., 1986, Am J Anat 177:161-185). The transcriptional characterization will initially be undertaken using Affymetrix hg-u133+2 GeneChips (Human Genome U133 Plus 2.0 Array, Affymetrix, Inc., Santa Clara, Calif.), as they will effectively cross-hybridize with Rhesus cDNAs (Chismar et al., 2002, Biotechniques 33:516-518, 520, 522; Wang et al., 2004, Nat Genet 36:687-693) and Rhesus chips from Affymetrix (GeneChip® Rhesus Macaque Genome Array). While the saturation of the genome using the Rhesus chip will be less than that obtained using human chips, it will nevertheless be a critically valuable resource, because of the anticipated lower noise levels owing to higher stringency that can be maintained using species-specific hybridization conditions. The comparison of human and Rhesus orthologs will be accomplished as described below.

Comparison of transcriptional patterns of pluripotent ESCs to determine their stage-specific similarity to pluripotent embryonic tissues: The next objective will be to generate transcriptional profiles for morula and inner cell masses of three or more Rhesus embryos. Epiblast cells from three or more gastrulation stage (day 13-14) Rhesus embryo will be obtained as described above. The transcriptional profiles obtained for these pluripotent embryonic cell types will be compared to transcriptional profiles of triplicate samples of three or more Rhesus embryonic stem cell lines. These will be compared to pre-existing data for the human (see Examples II 1 and 2), in order to obtain an estimate of the relative distances between the respective pluripotent cell types, using multi-dimensional scaling (MDS) and inter-group distance analysis (Wang et al., 2004, Nat Genet 36:687-693, see Examples II 1 and 2 and methods below). The comparison between human and nhpESCs and their pluripotent counterparts will address the hypothesis posed in this example, namely that primate ESCs resemble one of the native embryonic pluripotent cells, and clarify which pluripotent cell type is closest to ESCs.

Assessment of global transcriptional activity of imprinted genes in non-human primate embryonic and extraembryonic tissue lineages: An additional objective of the proposed transcriptional profiling of early non-human primate conceptuses will be to determine what stages and tissues express particular imprinted genes. The objective in this Example is not to determine the allele-specific origins of the transcripts, but simply whether the gene is transcribed at all, as the basis for subsequent imprinting experiments. There are to date over 70 known imprinted genes (http://www.geneimprint.com/databases/), of which a substantial fraction will be studied. However, the latter objectives will be greatly accelerated by having prior information about when and where expression occurs. While these genes will for the most part be represented on the Affymetrix hg-u133+2 GeneChip on the Rhesus GeneChip (Macaque GeneChip, Affymetrix).

### Detailed Methods:

hES cell culture: hES cells are maintained on a mouse embryonic fibroblast feeder layer in KO-DMEM (Gibco®, Invitrogen, Inc., Carlsbad Calif.) supplemented with 10% Serum Replacer (Gibco®) and 4 ng/ml basicFGF (R&D), or in feeder free conditions (Xu et al., 2002, Nat Biotechnol 20:1261-1264). Medium (KSR) is replaced daily, colonies are passaged by collagenase treatment 1 mg/ml in KSR, and subsequent mechanical dissociation. Embryoid bodies (EBs) are formed by detaching the hES cell colonies with collagenase treatment for 2-4 hours. The EBs are washed in PBS before plating in chemically defined medium (CDM; (Johansson and Wiles, 1995, Mol. Cell. Biol. 15:141-151) in Petri dishes. Day of plating is considered as day 0 for time courses. CDM composition is IMDMEM/F12 1:1, 1× lipid concentrate, 15 μg/ml transferrin, insulin 7 μg/ml, monothioglycerol 450 μM, BSA 5 mg/l or PVA 0.1% according to (Johansson and Wiles, 1995, id.). The concentration range of factors added is as follows: FGF-2 (0-50 ng/ml), BMP (0-4 ng/ml), LiCl (0-20 μM), Activin (0-4 ng/ml).

Transfection: Methods of transfection for hES cells are described by (Vallier et al., 2004, Stem Cells 22: 2-11); these will be used to produce transgenic lines expressing reporter genes under tissue specific promoters.

Analysis of gene expression: RNA is extracted by Trizol® (Sigma-Aldrich, St. Louis Mo.) and quality is monitored by the absorbance ratio at 260/280 nm and on denaturing gels as well as checked for absence of genomic contamination. For microarrays, concentration and quality of RNAs are further analyzed by Agilent bioanalyser (Agilent Technologies, Santa Clara Calif.). RT-PCR primers are designed to be specific for human and their sensitivity is optimized for low number of PCR cycles kept consistent for all experiments. To localize cells transcribing specific genes by in situ hybridization, the cDNA of the genes of interest will be synthesized by RT-PCR, purified from agarose gel and cloned in TOPO® vector (Invitrogen, Carlsbad, Calif.). The protocol for probe labeling and whole mount in situ hybridization from (Streit and Stern, 2001, Methods 23:339-344) will be used on hES cells and on EBs fixed in methanol and kept at −20° C. For localization by Western blotting, proteins are extracted with Trizol® using a cocktail of proteinase inhibitors. After separating by SDS PAGE, proteins are transferred onto nitrocellulose membrane by western blotting. Membranes are incubated with primary antibody and horseradish peroxidase conjugated secondary antibody is used for detection by chemiluminescent substrate. To analyze cells by FACS, EBs are dissociated by exposure for ˜30 min to Cell Dissociation Buffer (Invitrogen, Carlsbad Calif.) and further mechanical dissociation. GFP fluorescent cells are detected and quantified on a Becton Dickinson FACScalibur (Becton Dickinson, Franklin Lakes, N.J.), or when preparative amounts are needed, by cell sorting on a MoFlo FACS sorter (Cytomation, Inc., Fort Collins, Colo.).

Transcriptional profiling: For transcriptional profiling, either 1) the standard amplification protocol for Affymetrix arrays (Affymetrix, Inc., Santa Clara, Calif.) is used with large populations of cells (10⁵ to 10⁷) to generate labeled cRNA probes, starting from a total RNA amount of ≧1 μg; or 2) the amplification method described for single cell transcriptional profiling (Tietjen et al., 2003, Neuron, 38: 161-175) is used with small numbers of cells (10-1000) to generate sufficient cDNA for profiling. Affymetrix hg-u133+2 GeneChips are hybridized according to Affymetrix methodology.

A number of options are available with which to analyze the scanned chip images. The MAS (Microarray Analysis Suite) v5.0 package of Affymetrix and the RMA (Robust Multi-array Average) method of (Irizarry et al., 2003, Nucleic Acids Res. 31:e15) will be used in particular when converting chip images to transcriptional expression scores prior to further analysis. Briefly, MAS v5.0 will be used to assess technical/experimental quality control and RMA will be used to background correct, normalize and determine the absolute expression values of all individual elements (genes) on each chip. RMA is preferred to MAS v5.0 for the latter stage as published results (Irizarry et at., 2003, id. and others) show it to provide a more stable and repeatable representation of gene expression as captured by the Affymetrix GeneChip.

A host of state-of-the-art marker identification methods will be employed when analyzing array data for differential expression. Among the available options are Bayesian statistics (Smyth, 2003, Statistical Applications in Genetics and Molecular Biology 3(1), Article 3), variable fold-change methods (Quackenbush, 2002, Nat. Genet. 32 Suppl. 496-501) and the in-house method of Trotter et al. (2004, id.). Computational machine learning methods will be applied to microarray data using project microarray data. Thus, novel methods for marker gene identification will be applied to project data.

Data visualization and functional clustering will be performed on project microarray data, using techniques such as multi-dimensional scaling (see Example II 1) and hierarchical clustering, available via third party software packages such as dChip (Harvard University, Cambridge Mass.). Visual analysis is a great aid in discerning consistency in biological repetitions and in identifying clusters of genes that behave differently from the norm.

Single/Oligo Cell transcriptional analysis: The analysis of gene expression in small tissue samples will be carried out essentially as described by (Tietjen et al., 2003, Neuron 30:65-78). Briefly, immunosurgically isolated (ICMs) or manually dissected (e.g., single ESC colonies, epiblast, extraembryonic or primary germ layers) are obtained as pure tissue fragments, washed in PBS, then placed in a thin walled PCR tube containing 5 μl of ice cold cell lysis buffer (50 mM Tris-HCl [pH 8.3], 75 mM KCl, 3 mM MgCl₂, 0.5% NP-40, containing 80 ng/ml pd(T) 19-24 [Pharmacia, Pfizer, NY, N.Y.], 5 U/ml Prime RNase inhibitor [Eppendorf, Westbury N.Y.], 324 U/ml RNAguard [GE Healthcare, Piscataway, N.J.], and 10 μM each of dATP, dCTP, dGTP, and dTTP). Lysis is subsequently performed at 65° C. for 1 min. First-strand cDNA synthesis is then initiated by adding 50 U of MMLV and 0.5 U of AMV reverse transcriptases (Invitrogen, Carlsbad Calif.) followed by incubation at 37° C. for 15 min. Samples are heat inactivated at 65° C. for 10 min, and poly(A) is added to the first-strand cDNA product by-adding an equal volume of 200 mM potassium cacodylate (pH 7.2), 4 mM CaCl₂, 0.4 mM DTI, 200 μM dATP containing 10 U of terminal transferase (Roche Diagnostics, Basel, Switzerland) at 37° C. for 15 min. Samples are heat inactivated at 65° C. for 10 min, and the contents of each sample is brought to 100 μl with a solution made of 1× PCR buffer II (Applied Biosystems, Foster City, Calif.), 2.5 mM MgCl₂, 100 μg/ml bovine serum albumin, 0.05% Triton X-100 and containing 1 mM of dATP, dCTP, dGTP, dTTP, 10 U of AmpliTaq polymerase (Applied Biosystems), and 5 μg of the PCR primer AL1. The AL1 sequence is 5′-ATT GGA TCC AGG CCG CTC TGG ACA AAA TAT GAA TTC (T)24-3′ (SEQ ID NO: 8). PCR amplification is then performed according to the following schedule: 94° C. 1 min, 42° C. 2 min, and 72° C.6 min with 10 s extension per cycle for 25 cycles. An additional 5 U of Taq polymerase is added before performing 25 more cycles of PCR without the 10 s extension per cycle. In this manner, 10-20 μg of PCR-amplified cDNA can be synthesized from RNA of individual or small numbers of cells. Five microliter aliquots of each single-cell cDNA are checked for the presence of ubiquitous and cell type-specific markers by Southern blot hybridization. The objective of this step is to verify that sequences relevant to the tissue identity (e.g., Oct4, Nanog, Cripto in ESCs; Sox1, NeuroD1, nestin in neuroectoderm) are present in the amplification products, and that irrelevant sequences (e.g., epidermal cytokeratins) are absent. It is anticipated that the majority but not all of the amplified samples will qualify for further analysis on the basis of this criterion.

Laser capture microdissection: E15 to 20 rhesus embryos will be manually recovered from uteri (see Part V) and freshly embedded in Tissue-Tek OCT compound (Sakura, Torrance, Calif.). Then 5-7 pm parasaggital cryosections will be placed on permafrost-coated glass slides and taken through the following staining and dehydration procedure: 70% EtOH 30 s, H2O 5 s, Mayer's Hematoxylin (Sigma, St. Louis Mo.) 1 min, H₂O 5 s, Bluing Reagent (Thermo Fisher Scientific, Inc., Waltham, Mass.) 1 min, 70% EtOH 10 s, 95% EtOH 10 s, Eosin Y (Thermo Fisher Scientific, Inc., Waltham, Mass.) 20 s, 95% EtOH 30 s X2, 100% EtOH 1 min X2, xylene 5 min X2, air dry 15 min. Single or multiple cells will then be laser microdissected using a PixCell II Laser Capture Microdissection microscope (Arcturus, Sunnyvale, Calif.). The microdissected cells will removed from the Capsure caps (Arcturus) using a microneedle and placed into PCR tubes. Lysis buffer will be added directly to the laser-captured sample, which will be processed and checked by Southern blot for the strong expression of relevant genes as described above.

Tissue-tissue Comparisons in Human and Rhesus: Data from Affymetrix arrays will be processed using default parameters of the RMA model, as described above (Irizarry et al., 2003, id.). In order to directly compare human and Rhesus data captured on human arrays; a set of Affymetrix hg-u133+2 GeneChip probe-sets that respond to both human and Rhesus expression signal must be identified. Information regarding human-Rhesus probe equivalence is already at our disposal (Rob Norgren, University of Nebraska Medical Center, Omaha, Neb.) and the macaque GeneChip is available from Affymetrix. Further correspondence may be identified by a control study of pooled fetal or adult Rhesus tissues to establish a subset of human GeneChip array probes that can be reliably detected in both human and Rhesus samples.

Once a subset of array probes that describe both human and Rhesus gene expression has been identified, the normalized array data for that subset will be visualized in 2-13-dimensions and distance analysis will be performed on array profiles corresponding to the different tissue groups present. Expression profiles will be visualized using classical multi-dimensional scaling (MDS) (Kruskal, 1964, Psychometrika 29:1-27). MDS routines are available as standard functions of the Matlab (Mathworks, Inc., Natick, Mass.) and R (Department of Statistics and Mathematics, Wirtschaftsuniversität Wien, Wien Austria) statistical software packages (see Part IV).

In order to analyze the distance between groups of expression profiles and, in doing so, assess the similarity or otherwise between tissue groups, we will use the method described by (Wang et al., 2004, Nat Genet 36:687-693). Briefly, the distance between any two expression profiles is estimated using the Pearson correlation co-efficient subtracted from unity, to provide a bounded distance in the region (0,2). The distance between two groups of profiles, e.g. those that describe two types of tissue, is calculated using the average linkage (the mean of all pairwise distances (linkages) between members of the two groups concerned). The standard error of the average linkage distance between two groups (the standard deviation of pairwise linkages divided by the square root of the number of linkages) may be used to evaluate the significance of difference between two or more average linkage distances. Distance analysis of this nature will provide the basis for definition of relationships between the various pluripotent cell types that characterize early mammalian development, including hESCs.

### EXAMPLE II

### 5. Compare the Epigenetic Status of hESCs and Their in vitro Differentiated Progeny with the in utero Epigenetic Status of Non-Human Primate Lineages

The potential use of hES cells in cell-replacement therapies prompts a genuine concern about whether their in vitro differentiated progeny would possess the same differentiative stability as cell types arising in the course of normal development. Derivation and culture of mouse ES cells can perturb the expression and methylation status of imprinted genes (Dean et al., 1998, Development 125:2273-2282). Moreover, mouse embryos can undergo perturbation of imprinted gene expression as a consequence of exposure to certain culture environments (Doherty et al., 2000, Biol Repred. 62:1526-1535). This led us to hypothesize that human ES cells would also show perturbations of imprinted gene expression as a consequence of ESC culture and to predict that any loss of characteristic imprinted patterns of monoallelic gene expression would be accompanied by alterations in the methylation patterns of known imprinting control regions. To address this question, the status of imprinting in hES cells will be examined, both during passaging as undifferentiated, pluripotent cells and as they underwent differentiation in vitro and in vivo. Specifically, imprinted gene expression is to be examined at various times of cell culture (i.e., low, medium and high passage), both in ESCs and in differentiated cells derived from them.

In Example II 2, strictly monoallelic expression of all three paternally expressed genes was observed, both at moderate passage (50-60) and at high passage (75-100) numbers, implying the maintenance of normal genomic imprinting at this stage, thus appearing to refute the ‘imprint perturbation’ hypothesis. However, while monoallelic expression of the maternally expressed H19 gene was observed in moderate passage H9 cells, ‘relaxation’ of strict monoallelic expression of this gene was observed at high passages (>75) in one subline, and low levels of ‘silent’ allele expression in other maternally expressed genes, thus appearing to support the ‘imprint perturbation’ hypothesis. This asymmetric pattern of imprinted gene expression is striking because the disparity between paternally expressed and maternally expressed imprinted gene expression resembles that found in the Eed knockout of the mouse (Mager et al., 2003, Nat. Genet. 33:502-507). The tentative conclusion to be drawn from this small set of data is that imprinting of paternally expressed genes is stable, but that of maternally expressed genes is not (although two of the maternally expressed genes showed only low levels of expression of the ‘silent’ allele, and these levels were stable over long term culture). More precisely, the observation of a progressive increase in expression of the silent allele of the H19 gene until it became biallelic at ˜100 passages in one H9 subline leads to the tentative conclusion that hESC epigenetic status could be maintained in a normal configuration for a substantial period in culture, but that repression of the ‘silent’ allele could be compromised at high passage numbers. Thus, further studies will be performed to expand this characterization of the epigenetic status of hES cells, and to determine the basis for the loss of ‘silent’ allele repression of the H19 gene, expanding the analysis to other maternally expressed genes to determine whether this phenomenon is generally characteristic of maternally expressed imprinted genes.

The initial findings acquire additional significance in light of reports (Doherty et al., 2000, Biol Reprod 62: 1526-1535; Mann et al., 2004, Development, 131: 3727-3735) that repression of the paternal H19 allele of mouse embryos is affected by the embryo culture environment. Intriguingly, the results of (Mann et al., 2004, id.) show that onset of paternal H19 transcription was limited to the placental derivatives of the preimplantation conceptus. Since hESCs are a derivative of the inner cell mass, loss of their H19 gene repression would not be predicted from the mouse embryo findings, and indeed, H19 is similar to the other imprinted human genes that have been studied in hESCs in being predominantly monoallelic (H19 being strictly monoallelic at low passages). This suggests that the loss of H19 repression observed in hESCs could occur by a distinct mechanism from the loss of H19 repression in cultured mouse embryos (Mann et al., 2004). The ability to extrapolate from the mouse findings to human ESCs is limited however, by the scarcity of data concerning the status and mechanism of imprinted gene function in humans, consisting essentially of the expression and methylation status of Snrpn and its DMR (Geuns et al., 2003, Hum Mol Genet 12:2873-2879; Salpekar et al., 2001, Mol. Hum. Reprod 7:839-844). Therefore, Program-wide studies will be carried out, comparing imprinting in human ESCs and tissues of early postimplantation non-human primate conceptuses.

The epigenetic status of imprinted genes will be determined in undifferentiated ESC (both human and non-human primates) and their in vitro differentiated progeny. This will provide the background for an assessment of their expression during in vivo development in conceptuses of non-human primates.

Assessment of epigenetic status of hESCs and their differentiated progeny. The transcriptional status (monoallelic vs. biallelic) of imprinted genes will be further analyzed using SNPs to distinguish the products of the two parental alleles of human ESCs. There are now more than 75 human genes that could be candidates for this analysis. The expression of six imprinted genes (three maternally expressed and three paternally expressed) has already been analyzed, the question arises as to how many genes we should analyze as a representative sample. This depends on the degree of variability observed in the samples. As uniformly monoallelic expression of genes characteristically expressed from the paternal allele (maternally repressed) have been found, the case for investigating large numbers of such genes is diminished. Thus a limited number (˜3) of additional paternally expressed genes will be analyzed; candidates include SNRPN, GABRB3, MAGEL2, NDN, PEG1, PEG3 and SDHD. The observed variability is greater for maternally expressed (paternally repressed) imprinted genes, where low levels of ‘silent’ allele derepression were found in two genes (NESP55 and SLC22A18) and progressive derepression of the ‘silent’ allele of H19 in a single subline of H9 hESCs. On this basis, several additional hESC (≧5) lines that are informative for maternally expressed genes will be identified and studied, and differentiated progeny of hESCs in which such genes are expressed will also be studied. Candidate maternally expressed genes include MEG3/GTL2, CDKN1C/P57kip, p73, COPG2, ASCL2, KCNQ1DN, TSSC3, ZNF215, HTR2A, UBE3A, ATP10C and ElonginA3. It is important to point out that the objective of this research is not to characterize the expression status of all imprinted human genes, or even a majority of them. Rather, it seeks to identify the overall pattern of imprinted gene activity as a function of pluripotency vs. differentiative state, or as a function of culture history. Then the focus will be on defining the underlying mechanisms of whatever pattern is observed. The priority in selection of which of these candidate genes to analyze will be determined by the body of information available for the mouse embryo, the depth of equivalent information about their pattern of imprinting in the human, and the identification of polymorphisms in available hESC lines.

The effort to expand the number of informative genes by examining additional hES cell lines available will be aided by undertaking an analysis of the imprinted status of key imprinted genes in existing hESCs, including DNA and RNA extracts of numerous existing hESC lines listed by the NIH Stem Cell Registry (NIH, Bethesda Md.). This will enable a search for additional SNPs that can define allele-specific transcription in other hESC lines. Having established the set of informative imprinted genes, the effect of passage number will be examined as the variable most likely to influence the status of imprinted gene expression. Because of preliminary studies on hESCs at moderate passage numbers (50-60) indicate that their expression is monoallelic at these stages, high passage cells (75-100) will be examined for evidence of the onset of biallelic expression. A second variable is the identity (genetic and epigenetic individuality) of each hESC line; therefore each gene will be studied in duplicate lines whenever possible. A third variable is whether the expressed, imprinted gene is inherited maternally or paternally. Finally, any change in the status of imprinted gene expression as hESCs undergo in vitro differentiation will be determined. While no such changes have been observed to date, it will be important to continue to evaluate this issue in both monolayers of hESC-derived cells induced to differentiate by exposure to serum, and in embryoid bodies. These assessments will be complemented by studies analyzing methylation patterns in imprinting control regions in any imprinted gene(s) for which loss of characteristic monoallelic expression occurs.

Assessment of epigenetic status in early postimplantation stages of non-human primate conceptuses: This analysis will provide the groundwork for further studies in which normal nhpESCs or those having undergone perturbations in imprinting are studied to determine the mechanism(s) by which imprints are stabilized during early development. It will be important for this task to evaluate the imprinting status of both inner cell mass derived (i.e., fetal) and placental tissues; in order to provide the correct perspective for evaluating subsequent experiments. Specifically, it will be important to know whether placental tissues maintain ‘silent’ allele repression to a lesser extent than in fetal tissues, as in mouse embryos (Mann et al., 2004, Development 131:3727-3735; Shamanski et al., 1999, Hum Reprod 14:1050-1056), and whether there is any fetal tissue specificity in monoallelic expression of imprinted genes. The selection of tissues for analysis will be of ˜5 samples of each stage and tissue, these being the primary germ layers and their earliest derivatives (definitive mesoderm, endoderm and ectoderm, as well as lateral plate mesoderm, gut and neuroectoderm).

### Detailed Methods:

Allele-specific gene expression in hESCs and their differentiated progeny: For analysis of imprinted gene expression in hESCs and their differentiated progeny, similar procedures involving RFLP and DNA sequence polymorphisms will be used as for the data presented in Examples II 1 and 2. Briefly, in order to assess parent-specific expression of imprinted genes, informative polymorphisms between the two parental alleles of an imprinted gene will first be identified DNA is extracted from the hES cells, and the polymerase chain reaction (PCR) is used to amplify a fragment containing the putative polymorphism. Many of these polymorphisms are situated within a restriction site. In these cases, the PCR fragment is then purified and digested with the appropriate enzyme. Digested fragments are run on a gel (either agarose or polyacrylamide). Informative polymorphisms yield two bands after digestion, one reflecting the cut and the other the uncut fragment. To ensure the reliability of this approach, a control cell line, MRC-5 (fetal lung fibroblast) which was informative for the H19 polymorphism will be used. This line revealed mono-allelic expression as expected, and is used as a control in all restriction digests for H19, for example. To confirm the identity of all PCR fragments, and also to confirm the informative nature of H19, all PCR products are sequenced. For genes in which the informative polymorphism does not occupy a restriction site, identity of the hES cell transcript is determined by DNA sequencing of the PCR product.

Allele-specific imprinted gene expression in early postimplantation conceptuses of non-human primates: SNuPE reactions will be carried out as described (Shamanski et al., 1999, Hum Reprod 14:1050-1056; Szabo and Mann, 1995, Genes Dev 9:3097-3108). To quantify allele-specific expression, reverse transcription of the embryonic and extraembryonic RNA samples will be done as follows: reaction mix (1 μg RNA, 2.5 mM lower primer, 50 mM Tris-HCl pH 8.3, 75 mM KCl, 3 mM MgCl₂, 1 mM dNTP) is placed at 64° C. for 10 min, 42° C. for 15 min. 2.5 U/μl reverse transcriptase (RT; Superscript H, Invitrogen, Carlsbad Calif.) is added and the reaction continued at 42° C. for 15 min. The reaction is stopped by heating to 99° C. for 5 min, and finally held at 65° C. Controls without RT are done for each RNA sample to ensure that DNA is not amplified. Two microliters of cDNA are then added to a polymerase chain reaction (PCR) mix (20 mM Tris-HCl pH 8.4, 50 mM KCl, 1.5 mM MgCl₂, 200 mM dNTP, 0.5 mM lower and upper primers, 2.5 U/Rxn Taq DNA polymerase (Invitrogen). The reaction conditions are as follows: 94° C. for 30 s, 42° C. for 30 s, 72° C. for 2 min (40 cycles), 72° C. for 10 min followed by a 4° C. soak for H19, Igf-2 and Snrpn, and 94° C. for 45 s, 56° C. for 45 s and 72° C. for 1.5 min (40 cycles), 72 for 10 min followed by a 4° C. soak for other genes. PCR products are purified with Prep-Agene DNA purification Kit (BioRad, Hercules Calif.). Approximately 10 ng of PCR product is added to the SNuPE reaction (20 mM Tris-HCl pH 8.4, 50 mM KCl, 1.5 mM MgCl₂, 2 μCi of the specific [32P]dNTP, I mM SNuPE primer, 0.75 U Taq DNA polymerase) and the reaction proceeds as follows: 4° C. for 30 s, 42° C. for 30 s, 72° C. for 1 min. Electrophoresis through 12% polyacrylamide (BioRad) gels allows SNuPE reaction visualization by autoradiography and quantification with a PhosphorImager. Specific [32P]dNTPs added to each reaction are as appropriate to the polymorphisms in the parental alleles, as determined by DNA sequencing of imprinted Rhesus gene coding sequences.

The comparison of ART-derived versus in vivo fertilization will initially focus on IVF, with a minimum of three E15 or E20 conceptuses, which will each be dissected into their component extraembryonic tissues (chorion, amnion, ‘extraembryonic mesoderm) and embryonic tissues (definitive mesoderm, endoderm and ectoderm, as well as lateral plate mesoderm, gut and neuroectoderm).

### EXAMPLE II

### 6. Mechanisms Responsible for Maintenance of Epigenetic Status in Human and Non-Human Primate Pluripotent Stem Cells and Their Progeny

Having preliminarily established the pattern of expression of imprinted genes in hES cells and their differentiated derivatives, the methylation status of the relevant differentially methylated regions (DMRs) will be characterized using bisulphite sequencing (Olek et al., 1996, Nat Genet Nucleic Acids Res 24:5064-5066) as a means of defining the basis for the observed patterns of gene expression. In the specific case of the Igf2/H19 imprinted gene cluster, these two reciprocally imprinted genes share a common gametically imprinted enhancer located downstream of both genes. This “H19 DMR” is paternally methylated, resulting in paternal H19 silencing and IGF2 expression. After implantation, the parent-specific methylation spreads to the H19 promoter and exonic sequences. Recent evidence suggests that once this secondary methylation has occurred, methylation of the H19 DMR is no longer required to maintain maternal-specific H19 expression (Srivastava et al., 2003, J Biol Chem 278:5977-5983).

The first hypothesis to be tested is that the gametic imprint controlling imprinted expression of the H19 gene, the H19 DMR, progressively loses its methylation during hESC culture, thus accounting for the onset of ‘silent’ (presumptively paternal) allele transcription at high passage. Interestingly, at p48 the H19 DMR is still represented by both methylated and unmethylated sequences at a frequency not significantly different from 50% (X-square test, P>0.05; see Examples II 1 and 2), affirming that the normal gametic imprinting of this gene is still present at this stage. Significantly, localization of polymorphism in both the H19 DMR and the H19 coding sequence of the H9 hESC line enabled the determination that it is in fact the DMR-methylated allele that is silenced and the unmethylated one active in this line. Further analysis of methylation status by means of bisulfite results will test the hypothesis that loss of parent-specific gametic H19 DMR methylation is responsible for the subsequent onset of biallelic H19 transcription in the sub-line that exhibits biallelic expression at high passage. If this is not the case, the (secondary) methylation of the H19 promoter may not take place correctly in this H9 sub-line, thus allowing re-expression of the paternally silenced allele. Otherwise biallelic expression of H19 should be interpreted as lack of progression in the normal process of epigenetic silencing that occurs after fertilization, rather than as loss of imprinting per se (because the parent-specific gametic H19 DMR methylation remains unperturbed). This hypothesis is to be investigated with respect to the H19 DMR and also for other DMRs, together with analysis of allele specific expression of other imprinted genes. Other alternative mechanisms for disregulation of imprinted gene expression involve non-methylation-based mechanisms, such as loss of repressive chromatin modifications, like those mediated by Eed (Liang et al., 2004, Proc Natl Acad Sci USA 101:7357-7362; Mager et al., 2003, Nat Genet 33:502-507) or perturbation of RNA-based trans-activating silencing mechanisms (Davis et at., 2004, Curr. Biol. 14:1858-1862; Georges et al., 2003, Trends Genet 19:248-252). Initially the focus will be on methylation-based mechanisms, pursuing alternative approaches as shown to be necessary by the analyses below.

Analysis of DNA methylation: For analysis of DNA methylation of imprinting control regions, bisulphite sequencing will be used, which provides detailed mapping of methylated cytosine residues in individual DNA strands, to determine the DNA methylation status of four key DMRs, two paternally methylated and two maternally methylated, in each of two ESC lines whose imprinted gene expression has been studied in Example II 4. This analysis will be performed for the DMRs responsible for regulation of any imprinted genes showing a significant departure from monoallelic expression (i.e., >5% of total transcripts derived from the ‘silent’ allele). The bisulfite sequencing approach allows for good reproducibility with very high sensitivity, thereby permitting robust analysis of very small amounts of cells. In addition to the H19 DMR, three other DMRs are proposed as targets for analysis: the paternally methylated IG DMR regulating DLK1 and GTL2; and the maternally methylated SNRPN DMR and Kv DMR, regulating Snrpn and the KCNQ1OT1 subdomains, respectively (reviewed by (Verona et al., 2003, Annu Cell Dev Biol 19:237-259)). The methylation status of these four DMRs will place the transcriptional status of their respective domains' coding sequences in perspective. Specifically, this analysis will enable conclusion about whether the gametic imprints are maintained throughout extended culture, and if this is the case, will result in attribution of any loss of ‘silent’ allele repression to alternative causes. This interpretation will be of particular interest with respect to any paternally repressed genes that are seen to undergo loss of silencing (i.e., H19).

On the basis of the preliminary results showing maintenance of the H19 DMR methylation, it is clear that no significant loss of methylation had occurred at this locus as a consequence of in vitro embryo culture to the blastocyst stage, nor in the process of hESC derivation and prolonged culture (at least up to p48). The further analysis in each case will involve an assessment of DNA methylation at any promoters known to depend for their methylation on the methylation status of the gametic imprints (H19 on the paternally methylated state of the H19 DMR;Gtl2 on the maternally unmethylated state of the IG DMR; Snrpn/Snurf on the status of the SNRPN DMR; and KCNQ1OT1 on the maternally methylated KV DMR. In each case, then methylation status of each DMR and their relevant sub-domains will clarify whether inappropriate methlyation per se can be attributed a role in disregulation (if any) of the respective imprinted gene. A parallel analysis using nhpESCs will serve to generalize the findings, and importantly, to establish the foundation for perturbation of regulatory mechanisms and assessment of its developmental consequences.

Any hESC or nhpESC sublines in which there is evident disregulation of imprinted genes will be considered particularly valuable resources for evaluating the consequences of such disregulation, especially if such (biallelic) imprinted gene expression patterns are stable throughout subsequent passages and during differentiation (see Preliminary Studies). Further analysis of these lines will be conducted as described below. In the event that biallelic transcription correlates with altered DNA methylation, either of the DMR or of gene-specific promoters, this will encourage further experimental approaches involving perturbation of the methylation status of the cells. This latter intervention will be accomplished through the use of DNA methyl transferase inhibitors, such as azacytidine, procainamide or zebularine (reviewed by (Egger et al., 2004, Nature 429:457-463). For this purpose, hESCs or nhpESCs will be grown at exponential phase, then exposed to normal ESC medium containing the methylation inhibitor 5-azacytidine. Based on prior studies in mouse ES cells (Hattori et at., 2004, Genome Res 14:1733-1740), an initial concentration of 1-5 μM 5-azacytidine for 2 days will be used to determine the extent and trajectory of inhibition. Treatments will be performed in triplicate, analyzing imprinted gene expression and changes in DMR and specific promoter methylation. Studies on mouse ES cells have indicated that once methylation is lost or removed, the cells remain devoid of imprints.

Assess developmental consequences of perturbation of epigenetic status in nhpESCs: The developmental consequences of any perturbations in nhpESC imprinted gene expression status, whether spontaneous or experimentally induced, will be accessed by transplantation to chimeras and analysis of developmental outcomes, including both imprinted status and global transcriptional identity. The first task will be to characterize any sublines of nhpESCs that have undergone alteration in their pattern of imprinted gene expression status. This will be accomplished in parallel with the analysis on hESCs. Once nhpESC lines or sublines have been identified that have characteristic perturbations in imprinted gene expression, these will be archived. The further analysis of their fate involves generating chimeras by blastocyst injection or morula aggregation. Once chimeras have been generated in vitro, they will be transferred to the Rhesus uterus for development to early postimplantation stages. Embryos will be recovered at E15-20 for subsequent analysis of contribution to various germ layers and lineages, and for transcriptional profiling using the markers as criteria for normality of particular tissue lineages.

The characterization of physical integration and transcriptional complexity of the ESC-derived component of chimeric tissues will be accomplished by marking the donor nhpESCs with green fluorescent protein (GFP). This gain-of-function approach has been developed extensively using lipofection to introduce vectors containing the non-toxic hrGFP gene (Stratagene, La Jolla Calif.) in a pTP6 vector containing a drug selection marker under IRES regulation into hESCs (Vallier et al., 2004, Stem Cells 22:2-11). This vector confers ubiquitous, stable GFP expression on hESCs and their differentiated progeny, without loss of pluripotency. Therefore, it is an optimal means of distinguishing the donor (nhpESC-derived) cells from the recipient embryo descendant cells. For analysis of physical integration, resultant chimeric embryos will be cryosectioned (as described above, laser capture methods), using immunohistochemistry with complementary (i.e., red) fluorescent secondary antibodies to identify particular tissues. Alternate sections will thus be available for analysis of transcriptional status using laser capture microdissection, with GFP fluorescence as the identifying characteristic of donor-derived progenitor cells. As an alternative, sorting of fluorescent cells using FACS could be used to capture donor nhpESC-derived differentiated cells after dissociation of chimeric embryos using Cell Dissociation Buffer (Invitrogen, Carlsbad Calif.). In either case, the pattern of incorporation and transcriptional fidelity will provide a means of assessing definitively the pluripotent status of nhpESCs, an undertaking that cannot be accomplished using hESCs, owing to practical and ethical constraints against the use of human cells and animal embryos for this purpose.

### Detailed Methods:

DNA methylation analysis: The protocol we use for bisulphite-based methylation analysis was adapted from (Olek et al., 1996, Nucleic Acids Res 24:5064-5066). hESCs are treated overnight with 1 mg/ml proteinase K (Qiagen, Crawley, UK), and the following day DNA is extracted using the Genomic DNA Extraction Kit (Promega, Southampton, UK). 2 μg of DNA is digested overnight with a restriction enzyme that does not cut within the fragment of interest, for example for H19 DMR analysis Apal is used. DNA samples are serially diluted and denatured by boiling, then freshly made NaOH is added to a final concentration of 0.4 M. After 15 minutes of incubation at 50° C., DNA is embedded in 2.5% LMP agarose (Cambrex, Wokingham, UK) to form beads at DNA concentrations of 100, 10 and 1 ng/bead. Beads are treated (in the dark) with 5 M sodium bisulphite (Sigma, Gillingham, UK)) 110 mM hydroxyquinone (Sigma) on ice for 30 minutes, incubated at 50° C. for 3.5 hours and washed thoroughly with TE and water. One bead is used per PCR. First-round PCR is run in 100 μl in the presence of 2 U BIO-X-ACT Taq polymerase (Bioline, London, UK), 1× manufacturer's buffer, 3 mM MgCl₂, 400 μM dNTPs and 1 μM primers. Second round PCR is run in 25 μl in the presence of 0.8 U BIO-X-ACT Taq, 1× buffer, 2.5 mM MgCl₂, 400 μM dNTPs and 1 μM primers. PCR conditions for both rounds are five cycles at 94° C. for 1 minute, 50° C. for 2 minutes and 72° C. for 3 minutes, followed by 30 cycles of 94° C. for 30 seconds, 50° C. for 2 minutes and 72° C. for 90 seconds. PCR products are subcloned using the TOPO TA cloning kit (Invitrogen, Paisley, UK) and sequencing. Our standard for successful bisulphite conversion is 95% converted non-CpG associated cytosine residues; a minimal level (5%) of non-converted CpGs facilitates the identification of uniquely cloned (as distinct from repetitively cloned) sequences. At least 2 independent bisulphite treatments are performed, with analysis of between 10 to 15 unique clones per treatment.

Chimera and teratoma analysis using nhpESCs: hrGFP-expressing nhpESC lines will be generated as described for hESC lines by (Vallier et al., 2004, Stem Cells 22:2-11) using Lipofection of the hrGFP pTP6 vector with either puromycin or neomycin as drug selection markers. The in vitro pluripotency of such lines will first be determined by generating embryoid bodies, assessing the diversity of cell types using RT-PCR with markers specific to the primary germ layer derivatives, and with transcriptional profiling, using the markers established previously. The analysis of embryos will initially be accomplished by sensitive, viable imaging of the recipient embryos, and then again after recovery at E15 to E20. The assessment of chimeras will include an overall inspection of integration of nhpESC-descendant cells into the recipient embryo tissues, as defined by uniform expression of tissue-specific markers using immunohistochemistry (e.g., Sox 17 for definitive endoderm; Sox 1 for neuroectoderm, and MyoD for definitive mesoderm), using GFP fluorescence to identify donor cell descendants. Teratoma imaging will be performed both with and without Gd-DTPA enhancement, magnetic resonance microscopy, and post-implantation visualization of SPIO-labeled stem cells, as well as optical and electron microscopy. In vivo MRI of SPIO-labeled HESCs implanted in the mouse brain, and <10,000 implanted cells are detectable. Receptor-mediated endocytosis and lipofectamine SPIO labeling procedures are presented above. Because of likely variability between ESC lines and individual embryos, at least two independent nhpESC lines will be examined for each aberrantly expressed imprinted gene, or experimental perturbation, and triplicate embryos for each line.

### Part III

### Derivation and Safety Testing of Non-Human Primate Embryonic Germ Cell Lines

Two types of pluripotent stem cells, embryonic stem (ES) cells and embryonic germ (EG) cells have been developed from human and murine embryos and fetuses. ES cells are derived from the pre-implantation blastocyst whereas EG cells are derived from primordial germ cells (PGCs) in the fetal gonad. Such pluripotent stem cells are capable of differentiating into a wide variety of cell types and represent an important new resource for the treatment of human diseases. Differentiated cells produced from pluripotent stem cells could potentially be used to treat a wide variety of human diseases including Alzheimer's, Parkinson's, diabetes, stroke and heart disease. But major questions about the growth, normal differentiation, stability of genomic imprints and potential for tumor formation of stem cells need to be addressed before they can be used clinically. Technical and ethical barriers preclude many of these questions being addressed directly using human ES or EG cells. Therefore, many of these questions will need to be studied in pre-clinical animal models. Differences in the growth characteristics, marker expression and gene imprinting status of murine and human ES and EG cells suggest that mice might not represent the most appropriate model for all pre-clinical studies. Studies of ES and EG cells in primate species closely related to humans could help fill gaps in knowledge concerning the utility and safety of cell-based therapies. Whereas ES cells have been developed from non-human primates (nhp), there have been no reports of attempts to generate EG cells from non-human primates. It is proposed herein to derive primate EG cells and to compare their growth, differentiation capacity and marker expression with existing primate ES cells and with human ES and EG cells. Primate ES and EG cell lines can then be used to analyze questions that cannot currently be addressed using human stem cells. These include whether such stem cells can differentiate normally into all cell lineages in the embryo, whether stem cells will form tumors upon transplantation and whether altered genomic imprints exist and if they present a significant barrier to stem cell transplantation. Such analysis of non-human primate ES and EG cell lines will fill the gaps in our knowledge of the usefulness and safety of pluripotent stem cells in cell-based therapies.

Pluripotent stem cells have two important properties. First, they are immortal and can be grown indefinitely in the laboratory. Second, they can be differentiated into all the cell types present in the animal body. In mammals three types of pluripotent stem cells have been identified and isolated into culture (reviewed in Donovan and Gearhart, Nature 414:92-97, 2001; Thomson and Odorico, Trends in Biotechnology 18:53-57, 2000). Embryonal carcinoma (EC) cells are the pluripotent stem cells of testicular cancers and are derived from primordial germ cells (PGCs) in the fetal gonad (Stevens, 1967). Embryonic stem (ES) cells are derived from the inner cell mass of the pre-implantation embryo (Evans and Kaufman, Nature 292:154-156, 1981; Martin, Proc Nat Acad Sci USA 78:7634-7638, 1981). Embryonic germ (EG) cells are derived from cultured PGCs isolated from the fetal gonad (Resnick et al., Nature 359:550-551, 1992; Matsui et al., Cell 70:841-847, 1992). EC, ES and EG cells of murine origin all share certain markers and properties but also differ in certain important respects (Donovan and Gearhart, 2001; Pera et al., J Cell Sci 113:5-10, 2000; Thomson and Odorico, 2000). For example, EC cells typically are aneuploid whereas ES and EG cells are karyotypically normal and also maintain a normal karyotype in culture. Moreover, when EC cells are injected into the blastocyst cavity of the pre-implantation embryo they can contribute to the somatic cell lineages but they do not contribute to the germline (reviewed in Donovan and Gearhart, 2001). In contrast both ES and EG cells form good germline chimeras. The ability of murine ES cells to maintain a normal karyotype and to enter the germline in chimeric animals allowed the manipulation of the mouse germline in a way that was unimaginable before their isolation. The creation of a wide spectrum of mutations in specific genes allowed the analysis of mammalian development in new and innovative ways. Like ES cells, EG cells also are able to form germline chimeras and have been used to manipulate gene expression in the germline (Labosky et al., Development 120:3197-3204, 1994; Stewart et al., Developmental Biology 161:626-628, 1994).

The development of human ES (hES) and human EG (hEG) cells with properties remarkably similar to their murine counterparts created an incredible new resource for the treatment of human disease (Thomson, Science 282(5395):1827, 1998; Shamblott et al., Proc Nat. Acad Sci USA 95:13726-13731, 1998). Differentiated cells derived from human pluripotent stem cells in the laboratory could be transplanted into patients to treat a variety of human diseases such as Parkinson's, Alzheimer's, muscular dystrophies etc. Although much has been written about hES cells, hEG cells have received less attention. However, there are major reasons why it will be important to develop nhp models to study EG cell lines. First, available data suggests that hEG cells may have all the same properties as hES cells in terms of disease treatment. Indeed, hEG cell-derived cells were the first karyotypically-normal human pluripotent stem cell to be used in any animal model to show proof of principle of the use of such cells to treat human disease (Kerr, Llado, et al., J Neurosci 23:5131-5140, 2003). Second, other than ES cells, EG cell lines represent the only other karyotypically-normal pluripotent stem cell type. Because of their shared pluripotency but distinct origin, comparisons of ES and EG cells could provide an incredible tool to study the mechanisms regulating developmental potency. Moreover, development of the same cell types from different species could allow the shared features of these cells to be defined. Transcriptional similarities identified between hEG and nhpEG cells will reflect fundamental similarities in their cellular programs, potentially providing critical insights into mechanisms regulating developmental potency. A fundamental understanding of developmental potency will advance our knowledge of many areas of biology including embryonic development, gametogenesis, pluripotent stem cell self-renewal, adult stem cell potential and nuclear reprogramming and could have widespread implications for disease treatment. Third, derivation of hEG cell lines is eligible for Federal funding. Therefore, if EG cell lines prove to be as useful as ES cell lines for cell transplantation they have the distinct advantage that new cell lines could be derived with Federal support. Fourth, the derivation of EG cells from PGCs is a remarkable similar process to the formation of EC cells from PGCs that occurs during the formation of testicular tumors. Testicular tumors are the most common form of tumor in young men. Studying the mechanisms by which PGCs form EG cells in different species could provide new information about mammalian gametogenesis and the etiology of testicular cancer.

But major challenges lie ahead before either ES or EG cells can be used for human disease therapy. Three main problems need to be overcome before these cells can be used clinically. First, techniques need to be developed for growing hES and hEG cells in the large quantities needed to produce the numbers of differentiated derivatives that would be required for transplantation. Second, techniques need to be developed for differentiating hES and hEG cells into specific cell types with a very high degree of purity. Third, transplantation methodology will need to be developed in order to allow differentiated cells produced in the laboratory to avoid immune recognition and to avoid tumor formation. Underlying these three major problems are several safety issues that will need to be addressed if pluripotent stem cells and their derivatives are to be used clinically to treat human disease.

The first problem to be overcome is to develop improved conditions for growth of hES and hEG cells. Although some improvements in conditions for growing ES and EG cells have been made, further improvements are likely to be necessary if such stem cells are to be used in cell-based therapy (Richards et at., Nature Biotech 20:933-936, 2002; Xu et al., Nature Biotech 19:971-974, 2001). A major problem is that we know so little about these stem cell populations in terms of molecules that are essential for their growth. These molecules are likely to include growth factors, growth factor receptors, cell adhesion molecules, gap junction proteins, cell-cell adhesion molecules etc. Moreover, the growth properties of the small numbers of existing hES and hEG lines may not fully represent the conditions that will be used to grow all potential human pluripotent stem cell lines. One powerful method for understanding critical growth mechanisms is to compare cells derived from different species. For example, major advances in understanding the fundamental and evolutionarily-conserved mechanisms that regulate the cell division cycle came from studies in a variety of species including yeast, flies, worms, toads, mice and humans. Similar comparative studies of pluripotent stem cell populations from different mammalian species could provide crucial information about the factors regulating both the growth and differentiation of human ES and EG cells that would be difficult, if not impossible, to determine by other mechanisms.

Although some of the factors regulating the growth and differentiation of murine ES cells have been identified, a growing body of evidence suggests that not all of those factors are relevant to human cells (see Burdon et al., Developmental Bio 210:30-43, 1999b; Burdon et al., Cells Tissues Organs 165:131-143, 1999a for examples). For example, growth of murine ES cells is dependent on leukemia inhibitory factor (LIF) activation of the signal transducer and activator of transcription-3 (STAT3) (Niwa et al., Genes and Devt 12:2048-2060, 1998). But LIF has no apparent effect on human ES cells (Mitsui et al., Cell 113:631-642, 2003; Pera et al., 2000). Human pluripotent stem cells grow slowly by comparison with their murine counterparts, have a high rate of apoptosis and show a propensity for differentiation (Donovan and Gearhart, 2001; Pera et at., 2000; Thomson and Odorico, 2000). Therefore, murine ES and EG cells may not be entirely useful models for studying the growth of human stem cells. Another approach would be to develop more human ES and EG cell lines that would be more representative of the genetic diversity present in the human population, but that approach is currently limited by Federal regulations. Therefore, one powerful method would be to develop the same stem cell populations from closely related species such as non-human primates. Understanding the most fundamental mechanisms that control the growth and differentiation of these cells could lead to improved conditions for controlling the growth and differentiation of stem cells in closely related species including humans. The development of nhpES cells preceded that of human ES cells (Thomson et al., Proc Nat Acad Sci USA 92:7844-7848, 1995; Thomson et at., Biol of Reproduction 55:254-259, 1996). The few experimental studies reported indeed suggest that nhpES provide important information on stem cell behavior that is distinct from that obtained from murine cells (Kawasaki et al., Proc Nat Acad Sci USA 99:1580-1585, 2002; Sone et al., Circulation 107:2085-2088, 2003). The development of non-human primate EG cell lines (see Example III 2) could provide an important new reagent for understanding stem cell growth and differentiation in species more closely related to humans.

The second major problem to be addressed concerns the ability to differentiate pluripotent stem cells into stable, fully functional differentiated derivatives. One major underlying safety issue to be addressed is whether transplanted cells, either stem cells or their derivatives, will behave appropriately when transplanted into host animals. In some studies, murine ES and EG cells introduced into embryos produced chimeras, that showed skeletal and other growth abnormalities (Dean et al., Development 125:2273-2282, 1998; Durcova-Hills et al., Differentiation 68:220-226, 2001). Therefore an important question is whether ES and EG cells are capable of forming stable, fully-functional differentiated cells such as neurons, astrocytes, myoblasts, cardiomyocytes, etc. This can be tested to some extent by examining the ability of stem cells to produce embryoid bodies in vitro and to form teratomas in vivo. But the best assay to test the ability of pluripotent stem cells to stably differentiate is chimera formation. In this. assay, pluripotent stem cells are provided with the appropriate temporal and spatial signals to differentiate into the full spectrum of cells present in the body. However, ethical considerations prevent this experiment from being conducted with human ES and EG cells. The production of nhpES and nhpEG chimeras could reveal potential drawbacks of primate pluripotent stem cells that would be difficult or impossible to uncover by other means. (See Example III 2 regarding the ability of NHP ES and EG cells to differentiate in these assays.) Like pluripotent EC cells, ES and EG cells can form tumors when placed in the appropriate environment. Therefore, another important question is whether ES or EG cells or their derivatives transplanted into host animals will form tumors. This question is also addressed in Example III 2.

A further important issue to be addressed concerns the stability of the differentiated state. Throughout development, gene expression is strictly controlled both temporally and spatially. Such controlled gene activity is central to the stability of differentiation and the orderly behavior of cells in tissues. During normal development, allele-specific expression from, a genetic locus can be controlled depending on the parent of origin of the chromosome (Feinberg et al., Seminars in Cancer Biol 12:389-398, 2002; Mann and Bartolomei, Genome Biol 3:REVIEWS 1003, 2002; Surani, Nature 414:122-128, 2001). This property, known as imprinting, is thought to play an important role in controlling cell growth and differentiation during development. Although the precise mechanisms that regulate genomic imprinting remain to be resolved, compelling evidence suggests that DNA methylation and histone modifications in part mark or maintain imprinted loci (Jaenisch and Bird, Nature Genetics 33:245-254, 2003). Some studies of imprinted loci in murine ES and EG cells suggest that both cell types can show abnormal imprinting of specific genetic loci (Dean et al., 1998; Humpherys et al., Proc Nat Acad Sci USA 99:12889-12894, 2002; Humpherys et al., Science 293:95-97, 2001). This has been interpreted as indicating that cells derived form ES and EG cells might have abnormal imprints and, therefore, would have abnormal growth control. That situation could cause transplanted cells to be unstable in terms of differentiation and to perhaps form tumors. Alternatively, studies of imprinting of cells derived from human EG cells demonstrated that they show monoallelic expression of specific genes (Onyango et al., Proc Nat Acad Sci USA 99:10599-10604, 2002).

The conclusion drawn from these latter studies is that hEG cells, unlike their murine counterparts, do not demonstrate abnormal imprints that would represent a barrier to cell transplantation (Onyango et at., 2002). Therefore, the conclusions of the mouse and human studies are at odds with each other, and raise the important question of whether there are significant differences in imprinting between murine and primate stem cells. But, the imprinting status of human ES and EG cells has been difficult to examine comprehensively because studies of gene expression from imprinted loci requires knowledge of the imprints present in the parents. The human embryos and fetuses from which the cells were derived were obtained under conditions approved by various Institutional Review Boards (IRBs) that prohibited knowledge of the identity of the parents (Pera et al., Human Reproduction 16:2187-2194, 2001; Shamblott et al., 1998; Thomson et al., Science 282:1145-1147, 1998). This fact makes definitive analysis of imprinting difficult, if not impossible, in human ES and EG cells. Such a problem would not exist with newly-derived nhp ES and EG cell lines. Example III 1 describes experiments that are designed to develop methodology for analyzing imprinting in non-human primates, and in Example III 3 to test the effect of culture conditions on imprinting status of NHP ES and EG cells. This data will allow one to test the consequences of impaired genomic imprints on stem cell behavior, which is determined in Example III 2.

Before fundamental scientific discoveries are applied to treat human disease they must undergo rigorous testing in order to determine if such treatments are safe for clinical trials in human subjects. Typically pre-clinical trials are conducted in animal models. To date a number of experiments have been carried out in rodent models that test the idea that pluripotent stem cells, or their derivatives, can ameliorate or cure disease phenotypes. Although the number of examples is small, so far the data are remarkable. For example, ES-derived lymphocytes transplanted into mice with severe combined immunodeficiency (SCID mice) were able to restore normal immune function and rescue the phenotype (Rideout et al., Cell 109:17-27, 2002). In a rat model of spinal cord injury, EG-derived cells were able to restore motor function to paralyzed rats (Kerr et al., 2003). Although these remarkable studies speak to the great promise that stem cell therapy holds, it is recognized that rodent models have limitations in terms of modeling human disease. For example, studies of pluripotent stem cells derived from mice have already indicated significant differences between those cells and the same cells derived from human embryos and fetuses (see discussion above). Major differences between murine and human pluripotent stem cells have been detected in growth characteristics and growth factor dependence, marker expression and imprinting: Primates have served as important models for a variety of human diseases including malaria, atherosclerosis, AIDS, diabetes and neurological and hematopoietic disorders (Herrera et al., Intl J Parasitology 32:1625-1635, 2002; Hewitson and Schatten, Reproductive Biomedicine Online 5:50-55, 2002; Hu and Dunbar, Current Opinion in Molec Therapeutics 4:438-490, 2002; Jenner, Parkinsonism and Related Disorders 9:131-137, 2003; Lowenstine, Toxicologic Pathology 31:92-102, 2003). The importance of studying human disease in animals closely related to humans was recognized by Congress in 1960 when it established the first Primate Center. In the studies described here the rhesus macaques is proposed as a model. The reproductive physiology of rhesus macaques is well understood and the Pittsburgh Development Center (PDC) has a highly proficient natural breeding program, establishing the first pregnancies using artificial insemination in rhesus monkeys in 2000 (Gabriel Sanchez-Partida et al., Biology of Reproduction 63:1092-1097, 2000).

### Preliminary Studies

In Example III 1 markers will be identified for non-human primate PGCs. This will be a pre-requisite for unequivocally identifying PGCs in vitro and for unequivocally following their growth and differentiation in culture. The choice of markers are based on previous studies and those of others that have identified markers of mammalian PGCs. The first part of this analysis involves histochemical and immunocytochemical staining of PGCs in vivo in the context of the developing gonad. To this end were isolated gonads from a single non-human primate fetus and sections of those gonads were cut to stain with antibodies to known mammalian PGC markers. An analysis of marker expression in the human gonad was performed, and it was found that human PGCs express both SSEA-1 and SSEA-4.

Since SSEA-1 and SSEA-4 are markers of mouse and human PGCs these data suggest that they will serve as good markers of non-human primate PGCs also and support the idea that one will be able to develop markers of PGCs in monkeys. In addition will be determined at what stage of development PGCs could be isolated from nhp fetuses, culture conditions developed for growing nhp PGCs in vitro.

Nonhuman primate fetuses have been isolated at different stages of development. It was determined that at day 30 post fertilization fetuses were morphologically equivalent to 10.5 days post coitus (dpc) mouse embryos. Next were isolated gonads from these fetuses, which were dissociated onto irradiated feeder layers of ST0 cells. After 2 days of culture the plates were fixed and stained for TNAP, the classical marker of PGCs in many mammalian embryos and fetuses. Many TNAP+ cells were detected and these cells had all the morphological features of PGCs. In addition were isolated primary fibroblast cells that can serve as potential feeder cells for deriving or maintaining nhp EG cell lines as well as providing a potential source of nuclei for nuclear reprogramming studies. Taken together these data suggest that an appropriate stage of fetus for PGC-isolation has been determined, and culture conditions that permit the survival of nhp PGCs have been determined. In addition, in the course of these studies was developed nhp primary embryo fibroblasts that can serve as feeder cells for stem cell populations as well as providing a source of nuclei for nhp nuclear transfer.

The imprinting status of genes in nhp PGCs will also be analyzed using PCR based techniques. This will require the isolation of highly purified populations of PGCs and isolation of DNA and RNA from those cells. Small numbers (2500) of PGCs from mouse embryos were isolated by fluorescence-activated cell sorting (FACS), RNA was isolated RNA and cDNA libraries were constructed from those cells. To test these libraries they were screened for genes likely to be expressed in PGCs (Oct4, C-Kit, Nanog) and those not expressed in PGCs but expressed in surrounding somatic cells (Kit Ligand, Liml, BMP8). Libraries that were found to express non-PGC genes were discarded since they were likely to be contaminated with somatic cells. These data demonstrate that cDNA libraries have successfully been constructed from purified populations of PGCs.

In Example III 2, the fate of either nhp EG cells or nhp ES cells will be followed in chimeras by making use of cells expressing green fluorescent protein (GFP) markers. GFP has been used in a variety of studies for following cell fate in living embryos. Previously were created murine and human ES cells expressing a GFP/Neomycin resistance gene fusion protein (GFPNeor) from the regulatory elements of the Oct4 promoter. The Oct4 gene is expressed in the totipotent blastomeres of the pre-implantation embryo, the inner cell mass and in primordial germ cells. A genomic clone (GOF18) contains all the regulatory elements required to recapitulate endogenous Oct4 expression and was used to drive expression of GFP. This construct was transfected into hES cells and allowed one to follow the fate of undifferentiated ES cells in culture.

In Example III 3 will be analyzed the imprinting status of EG cell lines derived from non-human primates. In order to carry out that specific aim, reagents were developed for analyzing the imprinting status of genes in nonhuman primates. It was decided to look for informative polymorphisms between both Rhesus and Cynomolgus monkeys. The genes analyzed included the Peg1 and Peg3 genes, Igf2, Snrpn and H19. These genes were chosen because previous studies in humans had shown that they represented imprinted loci, that is, they encoded genes that were differentially expressed depending on the parent of origin. Additionally, both examples were chosen of genes that were maternally imprinted and ones that were paternally imprinted. Primers were designed to analyze these genes in non-human primate samples based on similar studies in humans. Genomic DNA was isolated from both Rhesus and Cynomolgus monkeys and products were amplified for PegI, Peg3 and Igf2 but not for Snrpn or H19.

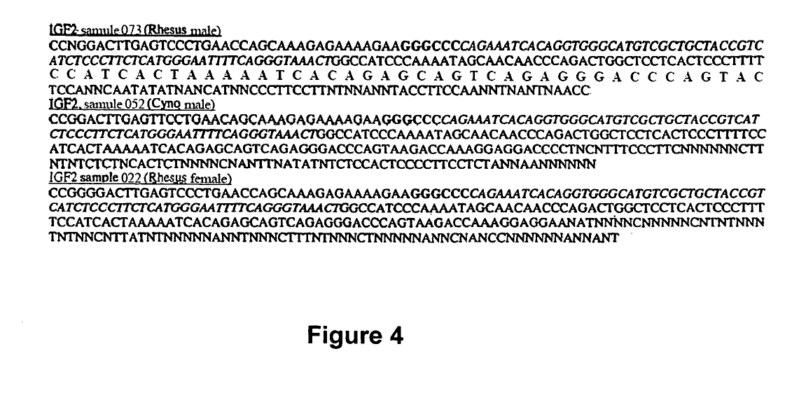

Analysis was performed with two Rhesus females, two Rhesus males, and three Cynomolgus males. In addition sequencing was done of the PCR products to ensure that the products were authentic (FIG. 4). In each case the PCR products had the appropriate sequence homology and were −97% similar to human sequences (data not shown). The degree of similarity of the non-human and human sequences is in agreement with the known degree of sequence conservation between these closely-related species. These data suggest that PCR primers have been successfully designed that can be used to analyze informative polymorphisms for the study of imprinting in non-human primates. Further, genomic sequences have been amplified from the DNA of the parents from which the nhpES cells were derived. Next was amplified cDNA from an nhpES cell line and it was found that these primers amplified the correct size fragment. Sequencing of the genomic DNA revealed a polymorphism in parental samples that could be used to analyze allele-specific expression. These data suggest that one would be able to identify informative polymorphisms in nhp genomic DNA and that these polymorphisms can be used to analyze allele-specific expression in nhp PGCs, nhpES and nhpEG cells.

### EXAMPLE III

### 1. Define the Characteristics of PGC Development in Non-Human Primates

Hypothesis: It is hypothesized that PGCs isolated from the gonads of non-human primates will express some, if not all, of the same markers as their counterparts from other mammalian species. It is further hypothesized that PGCs isolated from the gonads of monkey embryos and fetuses will survive in culture on feeder layers of irradiated STO cells in the same way as their mammalian counterparts. Finally, it is hypothesized that nhp PGCs will show similar changes in gene imprinting as their murine counterparts.

Rationale: A prerequisite for the analysis of PGC growth in vitro is the ability to unequivocally identify the cells in vitro and to be able to follow their fate in culture. To that end markers present on the cell surface of murine PGCs were examined in vivo and demonstrated that those same markers were expressed by PGCs isolated from the gonad (Donovan et al., 1986). Moreover, those markers could be used to unequivocally identify PGCs in feeder layer-dependent culture and to follow the fate of PGCs over time (Donovan et al., 1986). Since those markers are expressed by PGCs of other mammalian species including pigs and humans (Shamblott et al., 1998; Takagi et al., Molecular Reproduction and Devt 46:567-580, 1997) (and see Preliminary Data), it is expected that some of the same markers that have been used in the past to study PGCs in mice and humans should identify non-human primate PGCs.

The development of the mammalian germline seems likely to be an evolutionarily conserved process. Genes identified in mice as regulators of germline development have been found to be mutated in infertile human patients and vice versa. For example, mutations in the Deleted in Azoospermia (DAZ) gene were identified in human patients presenting with infertility. Subsequently, targeted mutation of a Daz-like (Dazl) gene in the mouse revealed an essential role for the murine Dazl gene in germ cell development (see (Cooke, Reviews of Reproduction 4:5-10, 1999; Fox and Reijo Pera, Molecular and Cellular Endocrinology 184:41-49, 2001) for reviews). Moreover, several germline markers are widely conserved within vertebrate and non-vertebrate species. The Vasa gene was originally identified in Drosophila as a gene involved in early germline development (reviewed in Raz, Genome Biol 1:REVIEWS 1017, 2000). The murine Vasa gene, mVasa, is involved in several aspects of germline development and marks PGCs from 11.5 dpc. onwards in the mouse embryo (Toyooka et al., Mech Dev 93:139-149, 2000). Vasa is also a germline marker in the Zebrafish and Chicken suggesting that this gene function has been conserved through evolution (Knaut et al., Current Biol 12:454-466, 2002; Krovel and Olsen, Mech Dev 116:141-150, 2002; Olsen et al., Mech Dev 66:95-105, 1997; Tsunekawa et al., Development 127:2741-2750, 2000). Taken together these data suggest that many of the molecular mechanisms controlling germline development have been conserved during evolution in mammals and also in vertebrates. Therefore, it seems reasonable to suggest that mammalian PGCs will share so-called lineage markers. This idea certainly holds true for other cell lineages. Here that concept will be examined by isolating PGCs from non-human primate embryos and testing first their ability to survive in feeder layer-dependent culture and secondly to express markers that are expressed on mouse or human PGCs.

Ultimately one of the goals of this project is to determine the safety and efficacy of pluripotent stem cells for transplantation in humans. Here these safety issues will be tested in a closely-related primate species. A major issue concerning stem cell safety is whether the imprinting status of imprinted genes is normal and, if not, whether that affects their ability to stably differentiate into specialized cells types. Genomic imprinting involves epigenetic modification of DNA in the germline that leads to preferential expression of a specific parental allele (monoallelic expression) in the offspring. An example of imprinting involves the gene for insulin-like growth factor II (Igf-2) encoded on human chromosome 11. The Igf-2 gene is active during early embryogenesis and is only expressed from the paternal allele (Vu and Hoffman, Nature 371:714-717, 1994). In contrast, H19, another imprinted gene also encoded on human chromosome 11 is expressed only from the maternal allele (Zhang and Tycko, Nature Genetics 1:40-44, 1992). Loss of imprinting of these genes producing biallelic expression has been observed in Wilm's tumor and cancer development, suggesting that altered imprinting could lead to developmental abnormalities. In both mice and humans, mutations that affect genomic imprinting can cause severe, sometimes lethal, conditions (Greally, Molec Biotech 11:159-173, 1999). Imprinting of genes is associated with changes in DNA methylation and may also involve changes such as histone modification (reviewed in Jaenisch and Bird, 2003). Such monoallelic expression is thought to play an important role in regulating embryonic and fetal growth (Reik et al., Novartis Foundation Symposium 237:19-31, 2001). Consistent with this idea, androgenetic and gynogenetic embryos in which both alleles are derived from a single parent, show significant developmental defects (McGrath and Solter, Cell 37:179-183, 1984; Surani and Barton, Science 222:1034-1036, 1983; Surani et al., Nature 308:548-550, 1984; Thomson and Solter, Genes and Development 2:1344-1351, 1988; Thomson and Solter, Developmental Biol 131:580-583, 1989). Similarly, ES cells derived from androgenetic embryos have very different growth properties and produce chimeras with skeletal abnormalities (Mann et al., Development 113:1325-1333, 1990; Mann and Stewart, 1991).

Some studies in mice suggest that there are differences in imprinting status between ES cells and EG cells while studies in human EG lines suggest that there are not. A problem with the studies utilizing human samples is that the investigators had no access to parental DNA and were therefore unable to definitively address this question. So a major question is whether the mouse data accurately represents the situation in human pluripotent stem cells. This question could be addressed easily in nhps where it is possible to study stem cells and the parents from which they were derived. A second major question is whether there are major differences in gene imprinting status between ES cells and EG cells and whether those imprints are normal. Again this question can only be addressed definitively if the imprinting status of the parental cell of origin is ascertained. For ES cells this requires examining imprinting status in the pre-implantation embryo, work that will be carried out in Part 1. EG cells are derived from PGCs during the fetal period. It is during this fetal period that genomic imprints are erased and then later re-established (reviewed in Arney et al., Intl J Developmental Biol 45:533-540, 2001; Davis et al., Human Molecular Genetics 9:2885-2894, 2000; Surani, 2001). Consequently, determining whether EG cells have appropriate imprints requires examining imprinting status in PGCs. Therefore, methods will be developed for studying genomic imprints in nhps and that technology used to begin to analyze imprinting in nhp PGCs. Moreover, this analysis will provide important new information about the regulation of genomic imprinting and gametogenesis in primates. Perturbations in the imprinting status of genes may lead to altered growth properties in the embryo or fetus, and are also be involved in the etiology of certain types of cancer including germ cell-derived teratomas and teratocarcinomas tumors (Tycko, Results and Problems in Cell Differentiation 25:133-169, 1999; van Gurp et al., J National Cancer Instit 86:1070-1075, 1994a; van Gurp et al., J National Cancer Instit 86:1070-1075, 1994b).

### Detailed Methods:

Biomarkers will be defined that can be used to unequivocally identify PGCs in vitro and to follow their fate. This approach is the same one that was employed previously to develop biomarkers and culture conditions for mouse PGCs (Donovan et al., 1986). Previously, PGCs were isolated from mouse, rat, porcine, bovine and human embryos and successfully placed into culture on feeder layers of irradiated STO cells (Donovan et al., 1986; Shamblott et al., 1998). Non-human primate PGCs should behave in the same way as their counterparts from the other mammalian species. In order to be able to identify PGCs in culture it is first necessary to determine what markers are expressed by PGCs in vivo. Based on previous studies in human, porcine and murine embryos, and based on differences in the length of gestation in these divergent mammalian species, it can be estimated that PGCs will enter the genital ridge (gonad anlagen) at around 30 days post fertilization. Previous studies also suggest that sexual differentiation will be complete in the rhesus testis by 45 days post fertilization (Hafez, J Reprod Fertil 2:294-295, 1971). In mice, PGCs isolated after the period of testis determination (detected by the presence of testis cords) are unable to form EG cells (Matsui et al., 1992; Resnick et al., 1992). However, in human embryos this was not the case, and EG cells lines were derived from gonads in which the testis cords were formed (Shamblott et al., 1998). Therefore PGCs will be analyzed from prior to 35 days and up to 45 days post fertilization. Embryonic fragments containing PGCs and gonads will be isolated from mid-gestation embryos and fetuses and either snap frozen or fixed in 2% paraformaldehyde in PBS. Sections from fixed gonads will be stained for markers known to be expressed on PGCs of other mammalian species using techniques developed in the lab.

The markers for which reagents are extant and which will be analyzed in this experiment are:
TNAP—tissue non-specific alkaline phosphatase, a marker of murine and human PGCs (Donovan et al., 1986; Shamblott et al., 1998);
mVasa—The murine homolog of the Drosophila vasa gene which is expressed in PGCs from 10.5 dpc (Toyooka et al., 2000);
Oct4—The POU domain transcription factor that is a marker of PGCs in all mammals tested (Yeom et al., Development 122:881-894, 1996);
SSEA-1—Stage-specific embryonic antigen-1—a carbohydrate differentiation antigen expressed on PGCs, EC cells, ES cells and EG cells of human and murine origin. (Donovan et al., 1986);
EMAI—a differentiation antigen expressed on murine PGCs from 11.5 dpc. (Hahnel and Eddy, J Reproductive Immunology 10:89-110, 1987);
CXCR4—A seven-transmembrane G-protein coupled receptor involved in PGC migration in species as diverse as Zebrafish and mice. (Ara et al., Proc Nat Acad Sci USA 100:5319-5323, 2003);
C-Kit—A receptor tyrosine kinase expressed on murine PGCs and shown genetically to be involved in PGC survival and proliferation. (Matsui et al., Nature 353:750-752, 1991);
GCNA-1—germ cell nuclear antigen-I (Enders and May, Developmental Biol 163:331-340, 1994);
Integrin Beta-1—a cell surface receptor for extracellular matrix proteins (Anderson et al., Development 126(8):1655-1664, 1999);

Based on previous studies in human, porcine and murine embryos, and based on differences in the length of gestation in these divergent mammalian species, one estimates that PG cells will enter the genital ridge (gonad anlagen) at around 32 days post fertilization.

Histochemical staining for TNAP will be carried out as described previously (Donovan et al., 1986). The reagents recognizing PGC antigens include rabbit polyclonal antisera and mouse monoclonal antibodies, all of which have been described previously. As controls, either pre-immune rabbit sera, isotype-matched control monoclonal antibodies or pre-incubate antibodies with excess antigen will be used. Primary antibodies will be detected with rhodamine- or fluorescein-conjugate secondary antibodies and staining observed using a Nikon microscope equipped with fluorescence optics. In controls, the secondary antibody will be omitted. Histochemical and immunocytochemical staining of gonads will allow the pattern of marker expression by PGCs to be determined.

Once the pattern of PGC marker expression in vivo is determined, Objective 2 will be addressed in which PGCs will be isolated into culture. For this objective, techniques will be employed that were developed previously for the culture of PGCs from murine and human embryos and which have also been used successfully to culture bovine and porcine PGCs (Dolci et al., Nature 352:809-811, 1991; Donovan et at., 1986; Resnick et at., 1992). Gonads will be isolated from non-human primate fetuses at or before 35 days of gestation.

Gonads are dissociated with trypsin/EDTA, and the resulting cell suspensions either centrifuged onto glass slides or plated onto feeder layers of irradiated STO cells in 96-well plates. Cytospin preparations will be air dried and stained for TNAP to determine the percentage of PGCs in the cell suspension. Cultured PGCs will be fed with complete medium (High Glucose formulation of DMEM supplemented with 15% fetal bovine serum) and LIF and bFGF. Twenty-four hours after isolation, one plate will be fixed and stained for TNAP activity using a standard protocol. TNAP positive cells will be visualized under a microscope. Cultures will also be fixed arid stained for other PGC markers using standard protocols. Briefly, cultures will be fixed with 2% para-formaldehyde in PBS or 1:19 Acetic Acid:Ethanol, washed three times in PBS, and incubated with antibody for 30 minutes at room temperature. Following antibody incubation, the cells are incubated with secondary antibody conjugated to either horseradish peroxidase or rhodamine isothiocyanite and observed under a microscope. The degree of PGC proliferation in culture will be assessed by incubating cultures with bromodeoxyuridine (BrdU). Detection of PGCs incorporating BrdU will be carried out using anti-BrdU antibodies and TNAP staining as previously described (Dolci et al., 1991). Incorporation of BrdU will indicate that PGCs are in S-phase of the cell division cycle and will allow one to assess the suitability of the culture conditions for PGC growth.

Imprinting analysis will be carried out as follows: briefly, expression from either the paternal or maternal allele will be examined by RT-PCR together with restriction enzyme digestion and DNA sequence analysis. To identify instructive polymorphisms between parental alleles, genomic DNA will be amplified using PCR primers designed for a variety of genes. These genes have been shown to be imprinted in mouse and humans and include IGF2, PEG1, PEG3, SNRPN, IPW and KCNQIOTI (also called LIT 1) which represent paternally expressed genes and H19, SLC22A18 (also known as TSSC5) and NESP55 which represent maternally expressed genes. These genes specifically will be studied because: i) previous studies have shown that they are either maternally or paternally imprinted, and the results can be compared with previous studies, ii) they are good examples of imprinted genes which show allele-specific expression in multiple tissue types (many imprinted genes are only imprinted in certain tissues and, therefore, not particularly good candidates for investigation) and finally, iii) there is good primer and sequence information available for these genes. Initially PCR primers will be used that have been used successfully to amplify the appropriate regions of the human genes, since non-human primates share 98% DNA sequence homology with humans. The list of genes provided below may however not reveal identical polymorphisms between humans and non-human primates, due to evolutionary divergence. In which case the primers will be used to amplify the appropriate region and sequence to search for new polymorphisms (see Alternative Approaches section—Examples III 1 and 3). The PCR primers used to analyze DNA polymorphism will be:

-H19—Silenced on Paternal Chromosome

Zhang Y and Tycko B. Monoallelic expression of the human H19 gene Nature Genetics 1992 1:404

| | |
|---|---|
| Forward primer: | |
| cggacacaaaaccctctagcttggaaa | (SEQ ID NO: 9) |
| Backward primer: | |
| gcgtaatggaatgcttgaaggctgctc | (SEQ ID NO: 10) |

With human genomic DNA this creates a band of about 700 bp, and human cDNA about 620 bp. After the DNA band is digested overnight with RsaI, an informative pairing would be indicated by one uncut band (700 bp) plus one cut band (about 550 bp). Allele specific expression is detected by repeating the RsaI digest using the cDNA band from an informative source. Bi-allelic expression would be revealed by two bands—620 bp and 450 bp, whereas mono-allelic expression would show just one of the two bands.

-IGF2—Silenced on Maternal Chromosome

Tadokoro K, Fujii H, Inoue T, Yamada M. Polymerase chain reaction (PCR) for detection of Apal polymorphism at the insulin-like growth factor II gene (IGF2). Nucleic Acids Research. 1991 19(24):6967

| | |
|---|---|
| Forward primer: | |
| cttggactttgagtcaaattgg | (SEQ ID NO: 11) |
| Backward primer: | |
| cctcctttggtcttactggg | (SEQ ID NO: 12) |

Same as above: DNA and cDNA create bands of about 300 bp. Digestion with ApaI cuts to 230 bp.

-PEG1—Silenced on Maternal Chromosome

Pedersen I S, Dervan P A, Broderick D, Harrison M, Miller N, Delany E, O'Shea D, Costello P, McGoldrick A, Keating G, Tobin B, Gorey T, McCann A. Frequent loss of imprinting of PEG1/MEST in invasive breast cancer. Cancer Research 1999 59(21):5449-51

| | |
|---|---|
| Forward primer: | |
| tactaaaccagcatacccttac | (SEQ ID NO: 13) |
| Backward primer: | |
| gcagtcatcataaaggaatcag | (SEQ ID NO: 14) |

DNA and cDNA create bands of about 310 bp. Digestion with AflIII cuts to 260 bp.

-PEG3—Silenced on Maternal Chromosome

Hiby S E, Lough M, Keverne E B, Surani M A, Loke Y W, King A. Paternal monoallelic expression of PEG3 in the human placenta. Hum Mol Genetics 2001 10(10):1093-100

| | |
|---|---|
| Forward primer: | |
| atgaatgcacagaaaccttcacttccag | (SEQ ID NO: 15) |
| Backward primer: | |
| ggtaagggtcaagtcctaggtgaaggtt | (SEQ ID NO: 16) |

Polymorphism 41 bp from end of ORF, in codon 1452. The frequent codon is cgc which can change to cac if polymorphic. No restriction site so just have to sequence and observe the peak at the polymorphic site. The surrounding sequence is: cagctcttcaatgaccGcctgtccctcgcca

-SNRPN—Silenced on Maternal Chromosome

Giacalone J, Francke U. Single nucleotide dimorphism in the transcribed region of the SNRPN gene at 15g12. Hum. Mol. Genetics 1994 3:379

| | |
|---|---|
| Forward primer: | |
| aaccaggctccatctactctttg | (SEQ ID NO: 17) |
| Backward primer: | |
| tcttgcaggatacatctcattcta | (SEQ ID NO: 18) |

DNA—about 1100 bp band, reduced to about 1000 bp when cut. cDNA—about 220 bp band, reduced to about 150 bp when cut. Use BstUI restriction enzyme.

-IPW—Silenced on Maternal Chromosome

Wevrick, R., Kerns, J. A. & Francke, U. Hum. Mol. Genet. 3, 1877-1882 (1994).

| | |
|---|---|
| Forward primer: | |
| gggaactcttctgggagtgaatgttatca | (SEQ ID NO: 19) |
| Backward primer: | |
| gggaggttcattgcacagaaatttgg | (SEQ ID NO: 20) |

DNA—is a 1550 bp band. cDNA—is a 868 bp band. Polymorphism is C to T

KCNQ1OT1—Silenced on Maternal Chromosome.

Lee, M. P. et al. Proc. Natl. Acad. Sci. U.S.A. 96, 5203-5208 (1999).

-SNP1 in H9

| | |
|---|---|
| Forward primer: | |
| cagccacctctgtggcgtgaatgttct | (SEQ ID NO: 21) |
| Backward primer: | |
| gctcaaacccgtctctgaaatgcacgg | (SEQ ID NO: 22) |

DNA and cDNA create bands of about 466 bp. Polymorphism is C to T SNP2 in H7.

| | |
|---|---|
| Forward primer: | |
| gatcctctccaggcagcttcttccaca | (SEQ ID NO: 23) |
| Backward primer: | |
| cataaggtaggtaagtitgtgtccctg | (SEQ ID NO: 24) |

DNA and cDNA create bands of about 268 bp. Polymorphism is G to A

-SLC22A18 DNA—Silenced on Paternal Chromosome

Onyango, P. et al. Proc. Natl. Acad. Sci. U.S.A. 99, 10599-10604 (2002).

| | |
|---|---|
| Forward primer: | |
| ctctcactgggcaaggccacct | (SEQ ID NO: 25) |
| Backward primer: | |
| gaggaggctgctccactcgctg | (SEQ ID NO: 26) |

DNA- is a 315 bp band. Polymorphism is G to A.

-SLC22A18 cDNA

| | |
|---|---|
| Forward primer: | |
| gccacttctcggaggaggtgct | (SEQ ID NO: 27) |
| Backward primer: | |
| ggagcagtggttgtacagaggcg | (SEQ ID NO: 28) |

cDNA- is a 231 bp band. Polymorphism is G to A

-NESP55 DNA- Silenced on Paternal Chromosome

Hayward, B. E. et al. J. Clin. Invest. 107, 31-36 (2001).

| | |
|---|---|
| Forward primer: | |
| ggctccttgtgctgtctgtcttgtag | (SEQ ID NO: 29) |
| Backward primer: | |
| ccacacaagtcggggtgtagctta | (SEQ ID NO: 30) |

DNA- is a 233 bp band. Polymorphism is T to C

-NESP55 cDNA

| | |
|---|---|
| Forward primer: | |
| tcggaatctgaccacgagca | (SEQ ID NO: 31) |
| Backward primer: | |
| cacgaagatgatggcagtcac | (SEQ ID NO: 32) |

DNA- is a 1141 bp band. Polymorphism is T to C.

To date, Preliminary Data suggest that most of these primers will successfully amplify primate DNA. Once polymorphisms between transcribed regions of genes in different animals are established, matings will be established to derive PGCs. PGCs will be examined for parental allele-specific expression using RT-PCR as previously described. Briefly, PGCs will be isolated at different stages of nhp development by fluorescence-activated cell sorting (FACS), magnetic bead separation or by single-cell sorting. Markers will have been developed for nhp PGCs. Some of these markers will likely be recognized by antibodies that can be used to sort PGCs by the techniques described above. Since one of the markers to be analyzed, SSEA-1, is expressed on both mouse and human PGCs it seems likely that anti-SSEA-1 antibodies could be used for cell separation of nhp PGCs as they have for mouse PGCs. RNA isolated from PGCs will be converted into cDNA, and then analyzed by sequencing of RT-PCR products. Previously, pools of 2500 mouse PGCs have been sorted for molecular biology analysis, so the numbers of cells that need to be isolated will not likely be prohibitive (see Preliminary Data). PGCs from nhp will be isolated at different stages of development both before and after their entry into the genital ridge. It is at these times that imprinting marks are thought to be erased from PGCs in mouse embryos. Preliminary Data demonstrates the ability to accurately stage nhp embryos and fetuses and that stages have been identified at which one can isolate PGCs.

Expected results and future studies: It is expected that some, if not all, of the antigens described will be expressed in nhp PGCs. Indeed some of the antigens described have already been shown to be expressed by primate (human) and other mammalian (Porcine) PGCs (Shamblott et al., 1998; Takagi et al., 1997) (and see Preliminary Studies). It is also expected that one will be able to determine at what stages of development PGCs can be isolated from nhp embryos and fetuses. It is further expected that the development of techniques for analyzing imprinting status of nhp PGCs should be straightforward since they rely on standard molecular biology techniques. The development of techniques for isolating and analyzing PGCs from a primate species closely related to humans could also lead to a fundamentally better understanding of gametogenesis in the embryonic and fetal periods. This in turn could lead to comprehensive knowledge of the risk factors that lead to infertility, sterility and the developmental of gonadal tumors. The imprinted genes chosen were used to demonstrate proof of principle and have been used successfully in other systems. Therefore, the data generated herein may be comparable with that generated in other mammalian systems. Other genes could be used in the future to analyze these questions and include: IPW, KCNQ1OT1, SLC22A18 and NESP55. Determination of the imprinting status of several genes in nhp PGCs could also provide the essential baseline for analysis of imprinting in non-human primate embryos and ESCs, including those derived from ART (IVF and ICSI).

Potential problems and alternative strategies: One potential problem is that one may be unable to identify non-human primate PGCs because they do not express any of the markers that are expressed on PGCs or other mammalian species. This possibility seems unlikely, since many of the markers are conserved in vertebrates as disparate as fish, mice and humans. Moreover, many of the markers chosen have already been shown to be expressed on human (primate) PGCs (see Preliminary Studies). Nevertheless, ongoing efforts to identify new PGC should produce new PGC markers that can be useful for this analysis. Cloning of non-human primate homologs of these genes should be straightforward since there will be a source of gonadal RNA from which one can amplify candidate genes. The ability to amplify non-human primate DNA sequences based on human sequence data should be straightforward and is demonstrated by the Preliminary Studies. A further problem may be that the antibody reagents available may not recognize the non-human primate antigens. To overcome this problem one can identify the peptide sequence of the non-human primate proteins and raise antibodies to that peptide sequence. Since the genomic sequence of humans is known in most cases, one will be able to amplify Rhesus or Cynomolgus cDNAs by simple homology-based PCR. Peptide antisera have been successfully raised to a variety of proteins by immunizing rabbits with an N or C terminal 15-mer peptide. Another potential problem is that the STO cell feeder layers may not support the growth of non-human primate PGCs. Again, this is a formal possibility but seems unlikely given the successful culture of human PGCs on STO cells. Nevertheless, in the course of isolating PGCs from non-human primate embryos, stromal cells lines have also been developed from the gonad region (see Preliminary Studies). Immortalized cell lines will be tested for their ability to support the survival and proliferation of PGCs. Such cell lines could also be useful for subsequent studies in which EG and ES cells will be differentiated into distinct cell lineages.

### EXAMPLE III

### 2. Derive nhp EG Cells and Compare Growth and Developmental Potential of Primate ES and EG Cells

Hypothesis: The hypothesis is that EG cells from non-human primates will have many of the same characteristics as existing non-human primate ES cells, namely pluripotency as demonstrated by a capacity for teratoma formation, and that they will moreover have a capacity for multi-lineage contribution to early postimplantation non-human primate conceptuses.

Rationale: EG cells of non-human primate origin should have many of the same properties as their murine and human counterparts (Thomson, 1998; Thomson and Marshall, Current Topics in Developmental Biol 38:133-165, 1998; Thomson and Odorico, 2000). These properties include cell cycle progression, response to sub-culturing, developmental potential and potential for tumor formation. EC, ES and EG cells of murine origin have been described as being pluripotent based on their ability to give rise to a wide variety of differentiated derivatives. Three widely-used assays have been used to test developmental potency; embryoid body formation in suspension culture in vitro, chimera formation following introduction into a donor blastocyst and teratoma formation in nude or histocompatible mice. EG cell lines developed from primates should, like their murine counterparts, be pluripotent. Although human EG cells have many features of pluripotent stem cells, ethical considerations preclude some of the in vivo testing of these cell lines that have been carried out with murine ES and EG cells. In fact it is illegal to introduce human ES or EG cells into human embryos. This fact effectively prevents a strict comparison of human ES and EG cells with their murine counterparts and, for all intents and purposes, also prevents one from making the conclusion that the human cells are truly pluripotent. There are no such ethical considerations surrounding the use of non-human primate EG cell lines, providing an important rationale for the development of nhp EG cells. To test developmental potency of the cells which will be developed, three distinct and commonly used assays will be used: embryoid body formation in vitro, chimera formation in vivo and teratoma development in nude or SCID mice.

### Detailed Methods:

Objective 1: Develop nhp EG Cells from nhp PGCs.

In Objective 1 monkey EG cells will be derived from PGCs. In Objective 2 of Example III 1 will be demonstrated that monkey PGCs can be cultured in vitro; the Preliminary Data suggests that this will be straightforward. PGCs will be isolated from embryos and fetuses of non-human primates and placed into culture as described above. Monkeys will be mated following hormonal stimulation of the female (Part V). Pregnancy will be followed by sonography (Part IV) and terminated between 28 and 45 days post coitus. In this experiment, the cultures will be supplemented with both LIP and bFGF, factors which are required for the formation of EG cells in mouse and human embryos (Matsui et al., 1992; Resnick et at., 1992; Shamblott et al., 1998). Cultures will also be supplemented with forskolin, a cAMP agonist, and one of the most potent PGC mitogens. PGC cultures will be fed on a daily basis and observed by Hoffman modulation optics to look for the appearance of colonies of EG cells. After 5-7 days, the cultures will be trypsinized and re-plated onto 24 well plates pre-plated with irradiated feeder cells. Each well of a 96 well plate will be transferred onto a single well of a 24 well plate. The cultures will continue to be fed daily for another 5-7 days, at which time colonies of EG cells should be apparent. Individual EG cell colonies will be isolated by trypsinization and manual isolation (picking), then transferred into a solution of trypsin/EDTA for 5 minutes at 37° C. Following trypsinization, individual colonies are dissociated manually with a micropipette and diluted in medium containing fetal bovine serum to inhibit trypsin action. The single cell suspension is then plated onto pre-formed feeder layers of STO cells. Cultures will be fed again on a daily basis and colonies of EG cells should be visible within 3-5 days after plating. EG colonies will be expanded and frozen down. Expanded colonies of EG cells will be tested for expression of markers expressed on EG cells or murine and human origin such as: TNAP, SSEA-1 and Oct4 (see above for details) and:

High Telomerase activity—a classical marker of pluripotent stem cells (Shamblott et al., 1998; Thomson, 1998);

SSEA-3—stage-specific embryonic antigen-3, a carbohydrate differentiation antigen expressed by human EC cells and nhp and human ES cells (Thomson, 1998)

SSEA-4—stage-specific embryonic antigen-4, a carbohydrate differentiation antigen expressed by human EC cells and nhp and human ES cells (Thomson, 1998)

TRA-1-60—a marker of human EC cells and nhp and human ES cells (Henderson et al., Stem Cells 20:329-337, 2002)

TRA-1-81—a marker of human EC cells and nhp and human ES cells (Henderson et al., 2002)

Staining will be carried out using standard immunocytochemical and histochemical techniques currently used in the lab (Resnick et al., 1992). Controls will include the use of pre-immune sera, isotype-matched control antibodies and omission of the secondary antibody. Telomerase activity will be measured using a commercially available TRAP assay according to the manufacturer's protocol. Human and murine ES and EG cell lines will be used as positive controls and commercially available somatic cells will be used as negative controls. To further characterize primate EG cells, karyotype analysis will also be done using standard protocols. Once nhp EG cell lines have been established, karyotype analysis will be carried out.

Objective 2: Compare the Growth Characteristics and Markers of Human and Primate ES and EG Cells.

To compare the growth properties of non-human primate EG cells with the existing non-human primate ES cells or human ES and EG cells a standard analysis of population growth, cell cycle kinetics, programmed cell death and efficiency of sub-culture will be done. To compare marker expression on non-human primate EG cells, standard immunocytochemical assays will be used. All of these assays will be carried out using many standard techniques.

To analyze population growth, simple assays of increases in cell number over time will be carried out. EG cells will be grown in feeder-dependent culture and cell number will be determined by counting dissociated cells in a hemacytometer. Because the feeder cells are irradiated and are quiescent, use may also be made of fluorometric assays for determining cell number. In addition, plating efficiency will be determined using similar techniques. Cell number will be determined in cultures before and after plating and the efficiency of cell plating determined.

Flow cytometric analysis of propidium iodide-labeled cells will be used to analyze cell cycle kinetics. Briefly, EG and ES will be separated from feeder cells by trypsinization at room temperature followed by unit gravity sedimentation. ES and EG cell colonies will be harvested and dissociated into single cells by trypsinization. Single cell suspensions will be fixed in ethanol, labeled with propidium iodide (PI), and analyzed by flow cytometry using standard techniques in use in the lab (Lincoln et al., Nature Genetics 30:446-449, 2002). To identify cells in S-phase of the cell division cycle more accurately, cells will be pulse labeled with BrdU, fix cells as above, and then stained with fluorescein-conjugated anti-BrdU antibody. To determine the rate at which cells progress through the cell cycle, cells will be pulsed with BrdU and harvested at intervals after pulsing. By comparing the BrdU-labeled and PI-labeled cells over time one can determine the rate at which BrdU-labeled cells transit through the cell cycle. To determine the rate of apoptosis one can examine the population of sub-G1 cells in the PI-stained population or carry out specific staining for markers of apoptosis such as Annexin V. Such assays are currently in use (Lincoln et al., 2002).

To compare marker expression by non-human primate ES and EG cells, immunocytochemistry will be done using antibody reagents recognizing stage-specific embryonic antigens (SSEAs) and other lineage-specific antigens. These include the Trafalgar (TRA) antigens (Draper et al., J Anatomy 200:249-258, 2002). Immunocytochemistry protocols will follow those routinely in use. Briefly, cultures will be fixed with 2% paraformaldehyde in PBS and then incubated with primary antibodies to SSEAs or other antibody reagents. Following incubation with the primary antibody, cultures will be washed and incubated with fluorescein- or rhodamine-conjugated secondary antibodies recognizing the first specific antibodies. Stained cells will be identified using a microscope equipped with fluorescence optics. As controls will be used pre-immune sera, isotype-matched control monoclonal antibodies or pre-incubation of antisera with antigen. As controls for the secondary antibody, use of the antibody will be omitted. As positive controls to demonstrate antibody reactivity will be used cells known to express those antigens such as mouse and human ES, EG or EC cells. Most of the reagents required for this analysis are readily available commercially.

Objective 3: Differentiate Primate ES and EG Cells Into Embryoid Bodies and Characterize the Spectrum of Resultant Cell Types Produced.

To assay the developmental potential of EG cells, they will be differentiated into embryoid bodies and those structures then allowed to differentiate further. Embryoid bodies will be generated from EG cell lines using well-established techniques (Shamblott et al., Proc Nat Acad Sci USA 98:113-118, 2001). Briefly, EG cell lines will be cultured on feeder layers and then passaged onto tissue culture dishes without feeder cells. After 3 days of feeder-independent culture the EG cells will be dislodged from the tissue culture dish and plated into non-tissue culture dishes. These cultures will be allowed to grow for several days in suspension until they form so-called cystic embryoid bodies in which the balls of cells begin to develop cavities. Some of the embryoid bodies will be harvested after 2 days of suspension culture and be plated back onto tissue culture dishes and allowed to differentiate further. By plating embryoid bodies onto gelatin-coated dishes one will enrich for endoderm derivatives. To examine the production of differentiated cells from EG cells, histological, molecular and immunocytochemical analyses will be done using standard techniques. In brief, the presence of differentiated cells will be examined initially simply by careful microscopic examination of growing cultures. Differentiated cells should have, in many cases, distinct morphologies from the undifferentiated stem cells. Next, cultures will be fixed with 2% paraformaldehyde and stained with antibodies to lineage specific antigens. For example, antibodies to intermediate filament proteins can be used to distinguish between cells of neuronal, glial and muscle origin. Antibodies to the intermediate filament protein neurofilament will specifically stain neurons, whereas antibodies to glial fibrillary acidic protein or desmin will stain glial cells and muscle cells respectively. Similarly, differentiation of EG cells into differentiated derivatives will be examined using RT-PCR analysis as described by Shamblott et al. (Shamblott et al., 2001). For example, differentiation into endoderm will be assayed by looking for expression of classical endoderm markers such as alpha fetoprotein, GATA4, and hepatic nuclear factor 3β. Production of muscle cells will be assayed by looking for expression of muscle-specific genes such as MyoD, Myf5 and Myf6 (Shamblott et al., 2001). Neuronal cell lineages, indicative of ectoderm differentiation, will be identified by looking for expression of neuronal markers such as nestin, neurofilament and Tau protein (Shamblott et al., 2001). Cardiomyocytes will be identified using RT-PCR and antibodies specific for recognition of cardiac differentiation such as cardiac muscle troponin I, desmin, atrial natriuretic peptide, anti-sarcomeric α actinin and anti-nebulin. These analyses will allow the spectrum of the developmental potential of nhpEGCs to be determined. Noncontracting EBs or other cell types will be used as controls. The ability of primate EG cells to differentiate into different derivatives will be directly compared in side by side experiments with the ability of nhpES cells to form differentiated derivatives.

Objective 4: Compare the Ability of nhpES and nhpEG Cells to Form Chimeras in vivo.

To test for chimera formation, nhpEG cell lines will be introduced into pre-implantation rhesus embryos as described in Part 1. Briefly, 4- to 8-cell embryos will be derived from hormone-stimulated rhesus macaques and collected by laparoscopy (as described in Core B). Approximately 10-15 EG cells will be introduced into each embryo as described in Part 1 and the embryos cultured briefly prior to embryo transfer into staged recipients (Core B): Females will be monitored until day 30 at which time the fetuses will be harvested for analyses (Core B).

For determining dynamic cell lineage allocation at the blastocyst stage following chimera formation, nhpESCs and nhpEGCs will be transfected with GFP-expressing constructs. This will allow noninvasive imaging of developing chimera embryos in vitro, as well as determination of EG and ES in chimeric pregnancies after amniocentesis. Such cells can be imaged in developing chimeras by high definition ultrasound (U/S) and MRI. This will allow one to follow the fate of groups of nhpEG cells in chimeric embryos and fetuses. Rhesus 8-cell embryos are prepared as above and a small clump of labeled or transfected nhpESCs or nhpEGCs are introduced into the well. After all reaggregations are completed, the plate is gently rotated to bring the fertilized embryos in close contact with the stem cells before returning to culture. All plates are checked the following day for chimera formation and embryonic development. Embryo transfer of nhpESCs+rhesus ♀♂chimeras is described in the Primate Animal Core. Dynamic imaging of cell tracer-labeled chimeric embryos using time-lapse video microscopy or a spinning microlens array confocal microscopy (UltraView; Perkin-Elmers, Boston, Mass.) will be performed as described in Imaging Part IV.

Another approach for marking cells and following their fate in chimeras is possible. One could generate stable nhpES or nhpEG cell lines expressing a green or red fluorescent protein marker in a constitutive manner. This can be achieved by transfecting or electroporating stem cells with a GFP or RFP marker driven by a constitutive promoter. One of such promoters, the Elongation Factor 1α (EF1α), works efficiently in hES cells. That construct will be modified to express GFP- and RFP-tagged proteins. As a reporter gene will be used a GFP-tagged deletion fragment of the Cyclin B1 gene, termed GFP-ΔCyclin B1. This construct accurately reports native Cyclin B1 localization and, importantly, unlike the full-length Cyclin B1, it's overexpression does not interfere with cell cycle progression. Because of cell cycle-dependent changes in Cyclin B1 localization, this construct can also be used to accurately monitor cell cycle progression in living stem cells. Therefore, one should be able to not only follow stem cells in chimeras but also ascertain whether they are dividing. Two methods of chimera formation will be investigated. First, biopsied embryos (obtained in Part V) will be microinjected with 8-10 GFP-labeled nhpESCs and nhpEGCs into the space vacated by blastomere withdrawal and the chimera's returned to BRL co-culture for embryonic development. A second method will employ re-aggregation of fertilized embryos with small colonies (10-15 cells) of nhpESCs and nhpEGCs carrying GFP transgene reporters (Part IV), and then counterstained with 5 μM propidium iodide (Molecular Probes, Eugene, Oreg.) to label the DNA. Fixed samples will be optically sectioned using a Leica TCS-SP2 laser scanning confocal microscopy as described in Imaging Part IV.

To test for contribution of nhpESCs and nhpEGCs to post-implantation stages, cultured blastocysts will be transferred to staged recipient females. Females will be monitored until day 30 at which time the fetuses will be harvested for analysis or the animals allowed to proceed to term (Part V). Analysis of chimera formation will also be carried out using genetic markers such as isoenzymes and mitochondrial DNA (mtDNA) polymorphisms (Dyke et al., Progress in Clinical and Biol Research 344:363-574, 1990; Hewitson et al., 2002; Holmes et al., Alcoholism: Clinical and Exptl Research 10:623-630, 1986; Khan, Genetica 73:25-36, 1987). For example, determining mtDNA polymorphisms in the Rhesus Colony will be accomplished by analyzing the D-loop of the rhesus monkey mtDNA genome through nested PCR and automated sequencing (Core B). Specifically, the hypervariable regions of the D-loop of the mitochondrial genome will be amplified from blood and or tissue samples. These products will then be sequenced using direct sequencing methods for mtDNA according to Hopgood et al. (Hopgood et al., Biotechniques 13:82-92, 1992). Unique polymorphisms will allow one to design specific primers for PCR amplification, a technique known as Allele Specific-PCR (AS-PCR). Alleles specific to certain animals will be identified by AS-PCR and resolved on 4% agarose gels. The mtDNA products from AS-PCR will be confirmed by restriction fragment length polymorphic (RFLP)-PCR analysis and Southern Blotting (St. John, 2000 #938). In the developing fetus and live offspring, the nhpESCs and nhpEGCs are expected to contribute to all three tissue lineages (endoderm, ectoderm and mesoderm) as well as the germ cells. Contribution to the germline can be monitored in the fetus by examining the fetal gonads and looking for donor-derived germ cells in the gonad. Isolation and culture of germ cells from fetuses could also be used to determine whether TNAP+ PGCs also express the GFP-tagged Cyclin B1 protein. All chimeric concepti will be monitored for defects in cranial, somite and limb development after ET by high definition ultrasound and MRI analysis. GFP transgene infected embryos will be examined by a brief exposure to attenuated epifluorescent illumination to confirm the extent of mosaic expression using appropriate fluorescein filters. Contribution of nhpES and nhpEG cells to the germline of animals after birth could be determined by biopsy of one of the gonads or, at a later stage, by mating. It is also expected that male stem cell lines introduced into female blastocysts will convert the embryo into a phenotypic and functional male as occurs in male/female murine chimeras. In this is the case, all of the offspring will be donor derived.

Objective 5: Determine the Ability of Primate ES and EG Cells to Form Teratomas in Nude Mice.

To test for teratoma formation and thus for in vivo pluripotency, EG cell lines will be injected intraperitoneally or intramuscularly into nude or SCID mice again using standard protocols (Thomson, 1998 #2). Briefly, 10×106 cells will be injected intramuscularly or intraperitoneally into nude or SCID mice and allowed to grow for approximately 60 days by which time palpable tumors should be present. Animals will be sacrificed and tumors removed for histology. Histological analysis will be carried out using standard techniques such as hematoxylin and eosin staining. Because murine and human ES and EG cells can from tumors when introduced into mice this raises the question of whether, during transplantation, the presence of contaminating ES or EG cells might eventually give rise to tumors. Injected animals will be allowed to age and monitored for signs of tumor formation. These will include development of palpable tumors at the injection site, altered behavior, altered eating and drinking, etc. Core A performs teratoma imaging both with and without Gd-DTPA enhancement, magnetic resonance microscopy, and post-implantation visualization of SPIO-labeled stem cells, as well as optical and electron microscopy. Receptor-mediated endocytosis and lipofectamine SPIO labeling procedures are presented in Core A. All experiments will be carried out under protocols approved by the IACUC.

Expected results and future studies: All of the experiments designed to study population growth, cell cycle kinetics and marker expression are straightforward and make use of well-established techniques. It is expected that nhp EG cells will share many properties with nhpES cells and with human ES and EG cells. It will be feasible to make nhpEG cell lines from a wide variety of animals in order to determine whether there are genetic influences on pluripotent stem cell behavior. This is relevant to the question of whether the data emanating from the existing human ES and EG cell lines is representative of the genetic diversity present in the human population. In the future one expects that nhp ES and EG cell lines will prove to be an excellent reagent for genome-wide analyses of gene expression by pluripotent stem cells. One would also expect chimeras formed between rhesus nhpEGCs or nhpESCs and rhesus ♀♂+♀♂ embryos to form normal blastocysts with proper ICM and trophectoderm lineages in vitro. It is anticipated that rhesus ES and EG cells will contribute exclusively to the ICM when reaggregated by either blastocyst injection or zona-free co-culture. Pregnancy in rhesus will be monitored by high definition ultrasound or MRI analysis. Amniocentesis and CVS will be collected. For morphological analysis of postimplantation embryos, paired control and chimeric concepti will be compared for defects in cranial, smite and limb development after embryo transfer or xenotransplantation of chimera into mice. The ability of chimera to form all three germ layers (ectoderm, endoderm and mesoderm) will be examined, noting deficiencies in their morphology and proper development. It is expected that xenotransplantation into SCID mice will prove to be a more useful tool in determining tissue development extent in nhpES and nhpEG cells ♀♂ because early concepti recovery is possible. Effect of disaggregation and reaggregation on the viability of newly created chimeras will be compared with the viability of non-manipulated controls. Chimeras will be placed in culture, their development monitored to the blastocyst stage, and their normalcy confirmed by cytogenetic analysis prior to initiation of embryo transfer into recipient females, SCID mice or isolation of embryonic stem cells. Embryo development will be evaluated by total cell proliferation, intercellular interactions, compaction, cavitation and blastocyst formation. It has already been demonstrated that embryo re-aggregation can result in viable rhesus offspring, as shown by the birth of Tetra (see Part 1). One expects that chimeras created from transgenic blastomeres will retain gene expression throughout preimplantation development. It is likely that embryo transfer of transfected chimeras will result not only in higher rates of transgenic offspring but also in offspring that will demonstrate germ line transmission of the transgene, as demonstrated in the birth of ANDi (Chan et al., Science 291:309-312, 2001). The data generated in this aim of stem cell behavior and potential will serve as direct comparison to data generated on imprinting status that will be generated in Example III 3.

Potential problems and alternative strategies: One potential problem is that one may not be able to derive nhp EG cells from Rhesus using the same conditions that are used to derive mouse and human EG cells. Two alternative approaches are possible. First, one can try to develop Rhesus EG cells using some of the growth factors that have been found to stimulate the growth of nhp ES cells and the factors that have been recently found to effect the growth of hES cells, such as Wnt3A, Nodal and BMP4 antagonists (see, for example, Vallier et al., Dev Biol 275:403-421, 2004). Secondly, one can attempt to derived EG cell lines from another nhp species, such as the Baboon. Some of the experiments are straightforward (population growth analysis, cell cycle kinetics, marker analysis, embryoid body and teratoma formation) and make use of well-established techniques. Based on preliminary evidence, one would not anticipate difficulties in generating tetraploid+fertilized chimeric rhesus embryos or culture in vitro. The combination of U/S and MRI will permit one to monitor the physiological parameters during early pregnancy establishment for deciding if and when to perform biopsies, terminate through an abortion, or to proceed with in utero development. The method for analyzing tumor formation by ES and EG cells may be difficult to interpret. It may prove difficult to distinguish between tumor tissue formed from stem cells and those derived from differentiated cells. To some extent this can be controlled by carrying out the mixing experiments thus described and by using unique retroviral integration sites as markers. In this technique, cells are marked with retroviruses whose unique integration sites serve as clonal markers of cells and their descendants. Another methodology is to mark stem cells with an Oct4-GFPNeo transgene. Expression of Oct4 is limited to pluripotent stem cells and cells of the germline and therefore serves as an excellent marker of these cell populations. Mouse and human ES cells were recently created expressing GFP from the regulatory elements of the Oct4 gene. The GFP marker is rapidly downregulated upon ES cell differentiation. Therefore, GFP serves as a marker of stem cells. Similar studies could be carried out with nhp ES and EG cells in order to follow the fate of true pluripotent stem cells in culture and in tumors since residual stem cells would express GFP.

### EXAMPLE III

### 3. Analyze the Role of Imprinting Status in Primate ES and EG Cell Lines on the Stability of Differentiation and Tumor Formation

Hypothesis: It is hypothesized that genomic imprinting in non-human primate EG cells will depend on their stage of derivation, with migratory PGCs more likely to have maintained imprints intact, and gonadal. PGCs more likely to have erased them. If the non-human primate EG cells were to follow the pattern seen in human EG cells, they would show normal, mono-allelic expression of the imprinted genes. However, if they were to follow the pattern seen in the mouse, they would likely to have initiated imprint erasure even before arriving at the gonad.

Rationale: Two aspects of imprinting are relevant to the study of pluripotent stem cells. EG cells are derived from PGCs during the fetal period. It is during this fetal period that genomic imprints are erased and then later re-established (reviewed in Arney et al., 2001; Davis et al., 2000; Surani, 2001). Some studies examining genomic imprinting in murine EG cells found that the cells had abnormal genomic imprints and biallelic gene expression, perhaps reflecting their derivation during the process of imprint erasure (Tada et al., Devt Genes and Evol 207:551-561, 1998). Consistent with these observations, some chimeras made from EG cells showed skeletal abnormalities resembling those associated with genomic imprinting problems (Durcova-Hills et al., 2001; McLaren and Durcova-Hills, Reprod, Fertility and Devt 13:661-664, 2001; Tada et al., 1998). Similar studies examining ES cells found aberrant allele-specific expression and methylation in ES cells and embryos derived from an interspecific cross (Dean et al., 1998). These alterations were not corrected during embryogenesis. Taken together, these studies suggest that altered genomic imprinting can occur in pluripotent stem cells. It has been suggested that the epigenetic state of ES and EG cells might make them unsuitable sources of cells for transplantation. In other words, if cells derived from ES and EG cells with imprinting abnormalities were transplanted into patients, they might grow abnormally or uncontrollably because of problems with genomic imprinting. This important issue has been difficult to examine in human pluripotent stem cells because the conditions under which the stem cells were derived included a specific ban on the investigators from having access to the tissue of the parents from which the embryos were derived (Onyango et al., 2002). Studies on imprinting in human EG cells concluded that, at the imprinted loci examined, the cells showed normal monoallelic expression in contrast to the studies carried out in mice (Onyango et al., 2002). However, because of the explicit ban on obtaining parental tissue it was impossible for the investigators to determine whether the cells expressed the gene from the correct allele or whether it had been reversed. Therefore, this problem will be examined in non-human primate stem cells. Because such stem cells can be derived from matings of identified parents, embryos (and subsequently stem cells) can be derived which carry easily-followed imprinting marks. EG cell lines derived from informative coatings can be differentiated and their differentiated progeny transplanted into suitable hosts. In this way, it will be possible to test whether pluripotent stem cells, and cells derived from them, obtained from non-human primates will behave correctly in transplant situations and whether differential genomic imprinting will have a significant impact on transplantation success.

### Detailed Methods:

In Example III 1 techniques will have been developed for imprinting analysis in non-human primates. The imprinting status of genes will be analyzed and compared in early embryos and derived ES cells and in PGCs and derived EG cells. The data on imprinting status of genes in the early period of development will be carried out in Part 1. Briefly, expression from either the paternal or maternal allele will be examined by RT-PCR together with restriction enzyme digestion and DNA sequence analysis. To identify instructive polymorphisms between parental alleles, genomic DNA will be amplified using PCR primers designed for a variety of genes. These genes have been shown to be imprinted in mouse and humans and include H19, IGF2, PEG1, PEG3, SNRPN, IPW, KCNQ10T1, SLC22A18 and NESP55. Initially PCR primers will be used that have been used successfully to amplify the appropriate regions of the human genes since non-human primates share extensive DNA sequence homology with humans. The PCR primers used to analyze DNA polymorphisms are described above (see Example III 1).

To date, preliminary data suggest that most of these primers will successfully amplify primate DNA. PCR products will be sequenced. Once polymorphisms are established between transcribed regions of genes in different animals, matings will be established to derive embryos and fetuses. Cell lines derived from these embryos and fetuses will be examined for parental allele-specific expression using RT-PCR as previously described. Briefly, RNA isolated from cell lines will be converted into cDNA, and then analyzed by sequencing of RT-PCR products. This analysis will be carried out on newly-derived EG cell lines and on existing non-human primate ES cell lines for which parental information is known.

To determine the effects of culture conditions on the stability of imprints, nhp ES and EG cells will be grown under different conditions and the effect of these different conditions on the expression of imprinted genes will be tested. Typically both hES and hEG cells are grown on feeder layers or mitotically-inactive cells. The nhpES cells are grown in these conditions also. Some studies report the growth of hES cells on Matrigel™, an extracellular matrix extract of sarcoma cells. Therefore, one of the conditions tested will be whether growth on Matrigel™ affects the imprinting status of nhpEG and nhpES cells. Another method commonly used to culture hES cells is to grow cells in the presence of serum-replacer rather than serum. Therefore, the effect of serum replacer on imprinting stability will be tested. Briefly, nhpES and nhpEG cells will be plated onto MEFs in serum or serum replacer or onto Matrigel™ and cultured for at least two “passages. Finally, the effect of growing nhpESCs in minimal conditions will be tested, as recently reported for hESCs (Vallier et al., 2004). They found that hESCs could be maintained in a pluripotent state for repeated passages in the chemically-defined medium of Johansson and Wiles (Mol Cell Biol 15(1):141-151, 1995) in the presence of recombinant (R and D systems) or transgenically expressed Nodal; this condition thus represents the minimal culture circumstances known to be compatible with maintenance of pluripotency, thus establishing a basal culture condition in which maintenance of ESC imprinting might be perturbed. Cells will be harvested and RNA isolated using standard methods in use in the lab. The cDNA will be constructed also using standard methods. Amplification of regions containing informative polymorphisms will be carried out using the methods described above (see Example III 1) and the PCR products sequenced. The sequence of the amplified fragments will be compared with that of the genomic sequences derived from the DNA of the parental samples to determine whether there is mono- or bi-allelic expression from that locus. This process will be repeated for as many genes as informative polymorphisms can be found. As a control for the effect of different culture conditions, experiments will also be carried out in which cells are treated with Azacytidine or Trichostatin, agents which would be expected to affect the expression of imprinted genes and which can cause reactivation of silenced alleles.

In Example III 2 the differentiation potential and tumor potential of nhp ES and EG cell lines will have been determined. In this specific aim will be determined the imprinting status of a number of imprinted genes and the two sets of data correlated. Briefly, the imprinting status of the battery of paternally- and maternally-imprinted genes in nhpES and nhpEG cell lines will be determined and compared with the degree of differentiation observed in the assays of developmental potency. For tumor assays will be determined the size of tumors, timing of tumor development from the time of inoculation, degree of differentiation into different lineages and percent of un-differentiated stem cells in tumors. These parameters will be determined using lineage specific markers as described in Example III 2. For in vitro assays will be carried out similar analyses of the ability of nhpES and nhpEG cells to differentiate into different cell lineages. Again lineage specific markers will be used, described in Example III 2. To test the ability of nhpES and nhpEG cells to differentiate, these cells will be transfected with the Oct4-GFPNeo construct, which can serve as an excellent marker of undifferentiated pluripotent stem cells.

Expected results and future studies: Based on Preliminary Studies it is expected that one will be able to find informative polymorphisms at imprinted loci in monkeys. The likelihood of finding instructive polymorphisms can also be increased by looking at different colonies of animals and by using different species. In the future one anticipates carrying out more in depth analysis of imprinted loci by examining methylation status at imprinted loci (Cui et al., Cancer Research 61:4947-4950, 2001). By establishing ES and EG cell lines from the appropriate nhp species one could carry out a very detailed analysis of the role of culture conditions on gene expression from imprinted loci. This type of analysis has important implications for understanding the usefulness and safety of pluripotent stem cells and of cultured embryos in vitro fertilization. Further, one expects to be able to correlate the imprinting data with the ability of nhp EG and nhp ES cells to differentiate, data which will have been derived in Example III 2. These data will be very informative in determining whether the behavior of pluripotent stem cell cells or their derivatives can be influenced by the conditions in which they have been cultured. In the long run it may be possible to manipulate the culture conditions to maintain correct imprint status and in doing so influence the correct behavior of cells in transplants. It is also expected that one will be able to determine the imprinting status of genes in PGCs derived from ART embryos and fetuses and to compare that data with data generated from normal PGCs, which will have been collected in Example III 1. These data will serve as an important comparison with the data generated from mice and other mammalian species.

Potential problems and alternative strategies: Because the human polymorphisms are unlikely to be conserved in non-human primates, the characterization of polymorphisms in the latter will require additional work. However, this task is a straightforward effort, and it is made easier by the availability of primer sequences for key imprinted genes (from prior publications or from Part II). Moreover, the generation of SNPs for imprinted genes is a key part of the development of this system for analysis of epigenetic gene regulation, and thus is to be carried out in this project to benefit the whole program. A bigger cause for concern would be that the genes known to be imprinted in the human and/or mouse might not be imprinted in the non-human primate. This could necessitate a search for polymorphisms in additional genes than those currently regarded as the ‘core set’ marked for analysis in all three projects. This would necessitate finding new informative polymorphisms in non-human primate imprinted genes. Based on human genome data one can predict gene structure in non-human primates and develop sequence information for coding regions of imprinted genes. Once one is able to analyze the exons of imprinted genes, one can search for animals carrying informative polymorphisms within exons that can then be analyzed at the RNA (transcript) level. To date however, genomic sequences have been amplified for several imprinted genes based on human PCR primers (see Preliminary Studies) suggesting that analysis of genomic imprinting in non-human primates is feasible. Another potential concern is the risk of not finding informative polymorphisms in the imprinted genes selected for analysis. However, it seems highly likely that there will be polymorphisms to identify each of the candidate imprinted genes, because there are over 200 animals within the PDC primate colony that can be screened in this way. Another approach will be to develop stem cell lines from inter-specific hybrids as has been carried out in the mouse. For example, Feinberg and colleagues developed EG cell lines from an interspecific cross of 129/SvEv and CAST/Ei mice (Onyango et al., 2002). Since these mouse strains are evolutionarily diverged they carry many polymorphic loci that can be used for the purposes of analyzing both genetic mapping and genomic imprinting. This same approach could be used in non-human primates. Thus one could generate embryos from matings of Rhesus (Macaca mulatta; 164 days gestation) and Cynomolgus (Macaca fascicularis; 167 days gestation) monkeys. Genomic sequence analysis would be used to search for polymorphisms within the coding regions of imprinted genes. Once those polymorphisms had been identified, matings from informative animals would be established. Rhesus monkeys and other macaques can be successfully mated and produce offspring that are viable and fertile. Such crosses could be used to generate embryos from which stem cell lines could be derived. Analysis of genomic imprinting in F1 hybrids derived from different species would follow the same principle as described above.

### Conclusions:

The Examples of Part III are designed to address fundamental questions concerning the safety and utility of pluripotent stem cells such as ES and EG cells. The development of pluripotent stem cell populations from primate species closely related to humans should accelerate efforts to develop cell-based therapy for the treatment of human disease. In addition, data will be generated concerning the development of the germline in primates. This data could significantly improve the understanding of gametogenesis in primates, including humans, and how that process can go wrong.

### Part IV

### Imaging Techniques

### EXAMPLE IV

### 1. MRI Related Techniques

Parts I, II and III above will all use the 4.7 T, and 11.7 T micro-imaging system and 4.7 T MRI system, and monkey and mouse model systems. The tissues of interest will be teratomas, kidneys, testes, subcutaneous regions, fetus and placenta. The MRI methods used will be 3D anatomical, Gd-contrast enhancement, MR microscopy and SPIO-labeled stem cells.

Animal Preparation for MRI

Mouse—Preparations will begin after the mice have been transferred to the NMR Center at least 24 hours prior to imaging. Anesthesia is needed to immobilize the animal during the MRI scan and to alleviate stress to the animal. The mouse will be anesthetized with isoflurane in air, intubated, and then connected to a mechanical ventilator (150 strokes/minute, 250 μl/stroke) delivering 1.25 % isoflurane in an oxygen/nitrous oxide mixture (70%: 30%). The mouse will be positioned in the bore of the 11.7 T micro-imaging system. Mouse physiology, including EKG, and pulse oximetry will be monitored in situ throughout the experiments. The animal's temperature will be maintained at 37° C. using a temperature-regulated collet surrounding the mouse. An imaging session will last a maximum of 3 hours. From previous experience, anesthetized animals can be routinely maintained in the vertical bore 11.7 T instrument for up to 8 hours and then safely recover the animal.

Monkey—Briefly, on the day of imaging the monkey will be sedated with ketamine (10 mg/kg) and transported in an environmentally controlled van from the Pittsburgh Development Center to the Mellon Institute. At the NMR Center, the monkey will be intubated, placed on a ventilator, and maintained on a mixture of isoflurane (0.5-2.0%) and 40% oxygen mixed with room air for the duration of the imaging procedure. Once anesthetized, a 23-gauge angiocath will be placed in a radial vein for administration of maintenance fluids and emergency drugs and an 8 French Foley catheter will be placed to maintain the bladder in an empty state. The monkey will then be placed on a heated pad in a supine position in the horizontal magnet. Heart rate, body temperature, and blood oxygenation will be monitored continuously to insure an adequate level of anesthesia; these measurements will be performed in situ in the magnet bore during imaging. A typical imaging session to last a maximum of two hours. During the primate imaging sessions, the laboratories containing the monkeys will be quarantined and restricted area signs will be posted. The only personnel allowed in the magnet area during the study will be the outside personnel from PDC accompanying the primate, the NMR Center's licensed veterinarian technician (LVT) and the MRI operator. All personnel will be required to wear gowns, mask, face shield, gloves, and shoe covers. After imaging, the monkey will be recovered from the anesthesia and then transported back to the Development Center while under sedation. After the completion of each imaging session, the LVT cleans the magnet bore and other surfaces that have been in contact with the primate with Quatricide/TB. A bite and scratch kit will accompany the primate for each study. Many additional details concerning the preparation, handling, and care of the primates during these procedures are outlined in the Part V.

3D Anatomical Imaging

Mouse—Mouse data will be acquired in the 11.7 T micro-imaging system. Either a 25 mm birdcage RF coil or a laboratory-built surface coil will be used for imaging localized regions of the mouse at high sensitivity. Motional image artifacts will be suppressed by triggering all data acquisitions at the same place in the respiratory cycle. Multiple, contiguous, two-dimensional (2D) slices (12) will be obtained along all three axes (axial, coronal, and sagittal) through the abdomen or ES cell implantation site. For the 2D imaging, a T1 or T2-weighted spin-echo pulse sequence with 256×256 or 256×128 image points will be used, and a resolution of approximately 80 micrometers and a 1-0.3 mm slice thickness. The acquisition time for a package of slices will be approximately 20 minutes. Alternatively, a rapid three-dimensional (3D) T2-weighted RARE (Rapid-Acquired-Relaxation-Enhanced) imaging sequence will be used. The RARE sequence works well at 11.7 T and will be used to acquire a 3D datasets in about an hour with 256×128×128 image points.

In a subset of experiments, an IV injection a Gd-based contrast agent (Omniscan, Berlex Laboratories, Montville, N.J.) will be used in conjunction with T1-weighted imaging. It will be investigated whether Gd-enhancement is beneficial for delineating teratomas. In these experiments, a dosage of 0.2-0.4 ml/kg will be delivered via the tail vein prior to imaging.

A total of approximately 50 mice/year will be imaged for using MRI. It will take approximately 1.5 hours/mouse to acquire the data, and approximately 2-4 mice will be imaged per day. The number of mice and scan times will be adjusted as needed.

Monkey—The monkey data will be acquired in the 4.7 T MRI system. This magnet has a bore size of just over 20 cm with the gradient and a volume RF coil in place. From past experience, in this system primates that are approximately <8.5 kg can be imaged. Several different RF coils will be used for these studies, including a birdcage volume coil, a laboratory-built hemi-cylindrical birdcage RF coil, and assorted surface coils. Throughout the course of these studies the RF coil technology will be refined to optimally-image the tissues of interest. Multiple, contiguous, two-dimensional (2D) slices (12-24) will be obtained along all three axes (axial, coronal, and sagittal) through the abdomen or ES cell implantation site. For the 2D imaging, a T1, T2, or T2*-weighted or diffusion-weighted spin-echo, gradient-echo, RARE, or EPI (Echo-Planar Imaging) pulse sequences will be used with a resolution of approximately 100-300 micrometers in plane, and a 2 mm slice thickness. Motional image artifacts from the mother will be suppressed by triggering all data acquisitions at the same place in the respiratory cycle via a breathing sensor/triggering circuit. If needed, fetal motion will be dealt with by acquiring navigator-echoes, and post-processing of the k-space data will be applied before Fourier transformation. As described above, an IV injection a Gd-based contrast agent will be used in a subset of experiments to determine whether Gd-enhancement is beneficial for delineating teratomas. In the monkeys, the agent will be delivered via the femoral vein at a dosage of 0.2-0.4 mL/kg prior to imaging. The 4.7 T scanner will be allotted 3-4 days per month for monkey imaging, and two animals will be imaged on that day. A total of 22 monkeys will be scanned per year. These numbers will be adjusted as needed.

MR Microscopy

From selected mice and monkeys with large teratomas (<1 cm), high-resolution 3D volumetric images of these cell masses will be acquired after they have been excised and fixed. This will allow the digitally ‘sectioning’ the data and performing ‘virtual histology’ (Ahrens and et at., 2002, Progress in Nuclear Magnetic Resonance Spectroscopy, 40:275-306; Johnson et al., 1993, Magn Reson Q 9:1-30) on the cell mass to examine the 3D structure. After the teratoma has been imaged in vivo, they will be excised and immersed in 4% paraformaldehyde in PBS for 24 hours at 4° C. The teratoma will then be embedded in agarose in a quartz tube and placed in a MR microscopy probe. Image data will be acquired using a T2-weighted spin-echo sequence, with 512×256×256 image points and an isotropic image resolution of approximately 50 μm. After imaging in 3D, they will be removed from the agarose and embedded in paraffin and sectioned using conventional histology. Side-by-side comparison of the histology with the corresponding in vivo and fixed MR image sections will be used to determine what the apparent MR features correspond to at the cellular level. It will require about 18 hours of imaging time to acquire 3D data for a single fixed teratoma. Approximately six teratomas per year will be imaged in this fashion.

MR Image Analysis

Data from all MR imaging sessions will be loaded into the software program Amira (TGS Inc., San Diego, Calif.). Using this software, the volumes will be edited to display the regions of interest. Other parameters will be adjusted as needed such as image intensity threshold, contrast, and image opacity in the case of volume rendered data. For images of teratomas (both in vivo and excised), the outer boundaries of the teratomas will be digitally segmented to examine the 3D structure. Teratoma boundaries will be traced using a combination of coronal, horizontal, and sagittal orientations. Starting from a user-defined seed point, boundaries are delineated using an AMIRA algorithm that selects contiguous voxels falling below a user-defined threshold intensity. Alternatively, the software will detect contiguous voxels falling within a user-specified range of intensities and seed point. The automated segmentation is then checked by eye slice-by-slice, and minor corrections can be applied using a stylus on a digital tablet (Wacom Technology Inc., Vancouver, Wash.). Using AMIRA, the pixel-to-pixel borders will then be smoothed in 3D to obtain the most accurate representation of each segmented structure. A 3D surface reconstruction of the teratoma will be created, and the volume will be automatically calculated. If possible, internal features of the teratoma will be segmented if there is sufficient intrinsic contrast differences in the tissue mass.

Cellular MRI Technology Development

A component of the imaging experiments will be to refine methods for labeling stem cells using MRI contrast agents for in vivo MRI. The undifferentiated cell types that will be used are mES cell 129/Sev (mouse), mES cell w95 (mouse), nhpES cell r366 (nonhuman primates), nhpES cell PDC-1 (nonhuman primate), hES cell H-1 (human) and hES cell HSF-6 (human).

Intracellular Delivery. In the proposed in vivo imaging experiments, stem cells that have been labeled in vitro will be injected. In order to obtain adequate sensitivity to follow the cells, a relatively large population of cells (>106) must be efficiently labeled with superparamagnetic iron oxide (SPIO) agents before implantation. An important preliminary step is to determine the optimum labeling protocol. Long-lasting cell labeling for in vivo MRI requires internalization of the SPIO particles. This involves an incubation period at physiological temperatures for several hours with excess SPIO particles added to the culture media. However, because stem cells have poor phagocytic properties, boosting the rate of intracellular delivery of SPIO will be investigated using a two-prong approach:

Addition of cationic lipids (e.g. Lipofectamine™) to the media

Utilization of mAb-SPIO complexes to promote receptor-mediated endocytosis (RME)

These approaches will be systematically investigated to determine which provides the greatest amount of labeling in the shortest incubation time without affecting pluripotency markers.

The SPIO agents will be obtained from commercial sources (Miltenyi Biotec Inc., Auburn, Calif.). These agents consist of an iron-oxide crystal (˜10 nm diameter) coated with polysaccharide resulting in a ˜50 nm diameter particle. Cationic lipids have been shown to be effective in the intracellular delivery of ES cells (Hoehn et al., 2002, Proc Natl Acad Sci USA 99:16267-72). The transfection agent Lipofectamine will be premixed with the SPIO agents for ˜20 minutes before adding to the culture media. 6 μl/ml of Lipofectamine provides effective coverage of the SPIO particles.

For the RME experiments, different mAbs will be screened to see which provides the greatest labeling efficiency. Table IV displays a tentative list of the cell surface carbohydrates expressed on stem cells that will be targeted with mAb. To rapidly screen the mAb among the list, SPIO agents that are conjugated to streptavidin (Miltenyi Biotec Inc., Auburn, Calif.). Biotinylated primary mAbs against the molecular targets molecules (Table 4) will be obtained commercially (Santa Cruz Biotechnology Inc., Santa Cruz, Calif.). Pre-saturation of biotin on cells will minimize non-specific labeling.

TABLE 4
Cell surface carbohydrates that will be targeted with mAb-SPIO
complexes for receptor-mediated endocytosis studies.

| mAb-SPIO | | Nonhuman | |
|---|---|---|---|
| Target | Mouse | Primates | Human |
| SSEA1 | X | | |
| SSEA3 | | X | X |
| SSEA4 | | X | X |
| TRA-160 | X | X | X |
| TRA-181 | X | X | X |

Immediately before labeling, cells will be harvested and labeled while in suspension. The incubation time and the SPIO particle concentration in the culture media will be systematically varied. A population of cells (˜10⁷ cells) will be subdivided into multiple sample populations (6-10). In one set of experiments, each sample population will be incubated with various concentrations of SPIO agent (in equally-spaced concentration increments) at a fixed incubation time (˜3 hours). In a second set of experiments, each sample population will be incubated for a different time period, ranging from 0.5 to 4 hours, at a fixed agent concentration. All incubations will be performed at 37° C. and in a humidified 5% CO2 atmosphere.

In either experiment, at the end of the incubation period, each sample population will be washed 2-times in culture medium to remove excess agents. The total number of cells will be re-counted using a hemocytometer in each sample population. In addition, the total number of dead cells will be estimated using the Trypan Blue exclusion assay. Next, the effective intra-cellular concentration of SPIO particles will be estimated in the remaining living cells in each sample population. To do this, the SPIO particles will be uniformly suspended in a known volume (˜50 μl) of agarose at the bottom of a small glass capillary tube. The longitudinal relaxation time (T2) of the capillary tube contents will be measured using a 20 MHz relaxometer (Bruker Instruments Inc., Billerica, Mass.). A control measurement will be made of cells in agarose that have not been incubated

From the measured T₂-values one can estimate the effective SPIO concentration per cell. The T₂ relaxation time is related to agent concentration according to the relation

1/T₂=1/T₂′+R₂[M] (1)

where T₂′ is the measured relaxation time in the non-incubated cell sample, R₂ is the relaxivity of the SPIO particles, and [M] is the net SPIO concentration of the cells in the agarose. The constant R₂ is a measure of the agent's effectiveness at relaxing spins; R₂ will be measured for our SPIO particles in solution in a separate experiment. From Eq. 1, [M] will be calculated, which is the only (unknown) quantity that will vary with incubation conditions. Dividing [M] by the total number of cells in each of the agarose gel samples will yield the effective SPIO concentration per cell or the cellular uptake of SPIO. Using this information several plots will be generated summarizing the results:

Effective cellular uptake of SPIO versus added SPIO concentration and incubation time

Labeling efficiency versus added SPIO concentration and incubation time

The quantity “labeling efficiency” is defined as the effective concentration per cell divided by the survival fraction (SF) of the cells. The SF is defined as

SF=(N₀−N_{dead})/N₀ (2)

where N₀ is the total number of cells prior to incubation, and N is the number of dead cells after incubation (determined by the Trypan Blue exclusion assay). Presumably, the plot of labeling efficiency versus incubation time will exhibit a maximum. The incubation time at this maximum, or the “optimum” incubation time, will be used for all subsequent labeling experiments for that specific cell type. Similarly, if the comparable plot for the added SPIO concentration exhibits a maximum in the labeling efficiency, the appropriate optimum concentration will be used for subsequent experiments. The other plots describing the effective cellular uptake will be used for our modeling studies.

Biological Effects of Labeling. It is important to confirm that SPIO labeling does not alter pluripotency of the stem cells. The results of Experiment 1 will determine a range of conditions providing efficient SPIO labeling using either the cationic lipids or RME.

As immunocytochemistry probes the heterogeneity of colonies, it will be used to determine the undifferentiated state of the murine, human, and nonhuman primate ES cells. ES cells will be probed for the positive ES cell markers: Oct-4, SSEA-3 and 4, Tra 1-80 and Tra 1-61, as well as the negative ES cell marker: SSEA-1. Immunocytochemistry will be performed in one or more of the following manners. 1.) Culture dishes containing undifferentiated colonies will be fixed by addition of either 100% methanol −20° C. for 15 min followed by a 15 min wash in PBS+1% Triton X-100 (PBS−Tx, Sigma, St. Louis Mo.) for 15 min, or 2% paraformaldehyde in PBS for 40 minutes followed by quenching in PBS+150 rnM glycine for 30 minutes subsequently washed out with PBS−Tx. After fixation, non-specific binding of the primary and secondary antibodies will be blocked when necessary by 20 minutes incubation in PBS with 3% non-fat dry milk. Primary antibodies are diluted in PBS+Tx and incubated on the coverslips for 40 min at 37° C. in a humidified chamber. After a 15 min. wash in PBS+Tx, an species appropriate fluorescently labeled secondary antibody is added to the coverslips for an additional 40 min. After another 15 min wash in PBS+Tx, 5 μM TOTO-3 (Molecular Probes, Eugene Oreg.) will be added for 20 min to label the nuclear DNA. Coverslips are inverted onto slides and mounted in Vectashield anti-fade medium (Vector Labs, Burlinghame, N.H.) to prevent photobleaching (Navara et al., 2001, Anticancer Drugs, 12:369-76). Slides will be examined using both conventional immunofluorescence and laser scanning confocal microscopy. Microscopes at the Pittsburgh Development Center will be used for these characterizations.

To confirm the intracellular distribution of the SPIO agents and that the cell morphology is unaffected by labeling, EM will be performed on labeled cells. Cells will be washed, pelleted, and fixed in PBS containing 2% glutaraldehyde at room temperature for 30 minutes and held overnight at 4° C. The cells will then be treated with 1% OsO₄ in PBS for 10 minutes. All of the samples will be washed three times in H₂0 and dehydrated in an ascending series of ethanol. Propylene oxide (PO) will be used as a transitional solvent. The cells will be infiltrated overnight in a solution containing a 1:1 mixture of PO and Epon-Araldite (EA). The next day the mixture will be replaced with 100% EA, and the sample was placed in a desiccator for 8 hours. The sample will be placed in plastic capsules containing EA and polymerized at 60° C. for 48 hours. Thin (0.1 μm) sections will be cut using a microtome and were placed on 200 mesh Cu grids. The samples were stained with 1% aqueous uranyl acetate and Reynolds lead citrate. Sections will be imaged using a transmission electron microscope. SPIO particles typically appear as a large number of punctuate dark spots within the cell.

MRI Sensitivity of Labeled Cells. A crucial issue when devising in vivo cellular MRI experiments is sensitivity limitations. Although MRI is a powerful tool for non-invasively mapping biological structures, it has a ubiquitous limitation in the available signal-to-noise ratio. A consequence of this limitation is that the ability to discriminate between tissues on the basis of differences in T₂ can be limited. Thus, when exogenous contrast agents are used to enhance T₂ differences between labeled and unlabeled cells, it is important to have an understanding of the minimum intracellular concentration and cell number per voxel that is required requirement to produce contrast. Although empirical results have demonstrated the feasibility of these labeling approaches for in vivo studies, the ultimate sensitivity of these methods is still not known.

Toward this goal, a simple mathematical model will be constructed for evaluating the minimal intra-cellular agent concentration and cell concentration per voxel required to produce “satisfactory” image contrast. The parameters describing MRI intensity and contrast are well characterized analytically. Starting with the basic equations used to describe the signal intensity in an MR experiment, the case of intracellular T₂ agents will be analyzed. The results of this T₂ contrast model will describe the minimal intracellular agent concentration and cell density required for satisfactory contrast enhancement (e.g. contrast-to-noise ratio>5). The model will take the form of a general set of equations that express this minimal concentration in terms of a small number of parameters that can realistically be estimated for a given experimental subject. These parameters will include intrinsic properties of the subject and agent, such as the “background” T₂ of the tissues of interest, the agent relaxivity, and the image signal-to-noise ratio of the targeted tissues. This model will be directly applicable to our proposed experiments. Using the results of the experiments which will determine the cellular uptake of SPIO, the density of labeled stem cells required for detectable image contrast in the targeted tissue will be predicted. This knowledge will be invaluable for predicting the success of future experiments. An analogous model for TI agents has previously been constructed which is described in the paper by Ahrens et al. (Ahrens et al., 1998, Proc Natl Acad Sci USA 95:8443-8). Before this model, strategies for addressing this question were based largely on empirical results for specific systems. The results of this model have proven to be extremely valuable in designing new types of ‘smart’ T₁ contrast agents that are sensitive to patterns of gene expression (Louie et al., 2000, Nat Biotechnol 18:321-5). MR images of the gel phantoms will be used to determine the minimum cell concentration that is needed to produce discernable image contrast.

### EXAMPLE IV

### 2. Light and Electron Microscopy Techniques

Immunocytochemistry will be preformed in one or more of the following manners: i.) Culture dishes containing undifferentiated colonies will be fixed by addition of either 100% methanol −20° C. for 15 min followed by a 15 min wash in PBS+1% Triton X-100 (PBS−Tx, Sigma, St. Louis Mo.) for 15 min, or ii) 2% paraformaldehyde in PBS for 40 minutes followed by quenching in PBS+150 mM glycine for 30 minutes subsequently washed out with PBS−Tx. After fixation, non-specific binding of the primary and secondary antibodies will be blocked when necessary by 20 minutes incubation in PBS with 3% non-fat dry milk. Primary antibodies are diluted in PBS+Tx and incubated on the coverslips for 40 min at 37° C. in a humidified chamber. After a 15 min wash in PBS+Tx, a species appropriate fluorescently labeled secondary antibody is added to the coverslips for an additional 40 min. After another 15 min wash in PBS+Tx, 5 μM TOTO-3 (Molecular Probes, Eugene Oreg.) will be added for 20 min to label the nuclear DNA. Coverlips will be inverted onto slides and mounted in Vectashield anti-fade medium (Vector Labs, Burlinghame, N.H.) to prevent photobleaching. Some of the antibodies tested for characterizing hESC nhpESC or mESC or for developmental staging of neuronal stem cell differentiation include the following:

TABLE 5
ANTIBODIES TESTED FOR CHARACTERIZING HESC
NHPESC OR MESC OR FOR DEVELOPMENTAL
STAGING OF NEURONAL STEM CELL DIFFERENTIATION

| Antigen | Location | Antibody | Source | Number | Dilution |
|---|---|---|---|---|---|
| β III | Early neuronal | Mouse | Covance | PRB- | 1:100 |
| tubulin | | monoclonal | (Princeton NJ) | 435P | |
| α- | Early endoderm | Mouse | Sigma (St. A8452 | 1:100 | |
| feroprotein | | monclonal | Louis MO) | | |
| Brachyury | Early mesoderm | Goat | Santa Cruz | SC17743 | 1:100 |
| | | polyclonal | (Santa Cruz | | |
| | | | CA) | | |
| CREST | Centromeres | Human | Gift- Dr. Cal | | N/A |
| | | Polyclonal | Simerly | | |
| Engrail-1 | Neurectoderm | Mouse | Univ. Iowa Dev. | 4G11 | 1:100 |
| | | Monoclonal | Bank (Ames IA) | | |
| H3 acetyl | Euchromatin | Rabbit | Millipore | 06-599 | 1:200 |
| lys9 | | polyclonal | (Charlottesville, | | |
| | | | VA) | | |
| H3 Di-Me | Euchromatin | Rabbit | Millipore | 07-030 | 1:100 |
| lys 43 | | polyclonal | (Charlottesville, | | |
| | | | VA) | | |
| H3 TriMe | Heterochromatin | Rabbit | Millipore | 07-030 | 1:100 |
| lys9 | | Polyclonal | (Charlottesville, | | |
| | | | VA) | | |
| Lamin | Nuclear laminae | Rabbit | Santa Cruz | sc-20681 | 1:100 |
| A/C | | polyclonal | | | |
| H2B N | Pan chromatin | Rabbit | Cell Signaling | 2575S | 1:50 |
| terminus | | polyclonal | (Danvers MA) | | |
| Lamin B2 | Nuclear laminae | Mouse | Invitrogen, | 33-2100 | 1:50 |
| | | monoclonal | (Carlsbad CA) | | |
| Lamin B1 | Nuclear laminae | Mouse | Invitrogen, | 33-2000 | 1:50 |
| | | monoclonal | (Carlsbad CA) | | |
| mushashi-1 | Neuronal | Rabbit | Chemicon | AB5977 | 1:200 |
| | progenitor | polyclonal | (Temecula, CA) | | |
| Myosin-1 | RNA pol \|\| sites | Rabbit | Gift - Dr. R. | | 1:100 |
| | | polyclonal | deLanerolle | | |
| Nanog | Pluripotent | Goat | R&D systems | AF1997 | N/A |
| | nuclei | polyclonal | (Minneapolis | | |
| | | | MN) | | |
| NCAM | Neural | Rabbit | Chemicon | CB5032 | 1:50 |
| | progenitor | polyclonal | | | |
| Nestin | Neural | Mouse | Covance | MMS570P | 1:100 |
| | progenitor | monoclonal | | | |
| Nestin | Neural | Rabbit | Abcam | Ab 7659 | 1:100 |
| | progenitor | polyclonal | (Cambridge | | |
| | | | MA) | | |
| Nuclear | Nuclear laminae | Mouse | Covance/Babco | MAb414 | 1:250 |
| porins | | monoclonal | | | |
| Oct-4 | Pluripotent | Mouse | R&D systems | MAB1759 | 1:100 |
| | nuclei | monoclonal | | | |
| Oct-4 | Pluripotent | Mouse | Santa Cruz | SC-5279 | 1:100 |
| | nuclei | monoclonal | | | |
| Oct-4 | Pluripotent | Rabbit | Active Motive | 39040 | 1:25 |
| | nuclei | poilyclonal | | | |
| Pax-6 | Neurectoderm | Rabbit | Covance | PRB 278P | 1:100 |
| | | polyclonal | Research | | |
| PE-CAM | Endothelial | MOuse | Chemicon | MAB2148 | 1:100 |
| | progenitor | monoclonal | | | |
| sox-1 | Neural | Rabbit | Chemicon | AB4768 | 1:200 |
| | progenitor | polyclonal | | | |
| SSEA-3 | Pluripotent stem | Mouse | Iowa | MC-631 | 1:25 |
| | cells | monoclonal | Hybridoma | | |
| | | | Bank | | |
| TRF-1 | Telomeres | Mouse | Abcam | AB1423 | N/A |
| | | monoclonal | | | |
| Tryosine | Dopaminergic | Rabbit | Pel-Freeze | P40101-0 | 1:200 |
| hydrolase | neurons | polyclonal | (Brown Deer, | | |
| | | | WI) | | |

Slides will be examined using both conventional immunofluorescence and laser scanning confocal microscopy. The Pittsburgh Development Center houses a Nikon E1000 upright microscope equipped with high numerical aperture objectives and ORCA Cooled CCD camera (Hamamatsu, N.J.). Laser scanning confocal microscopy will be used to enhance the imaging using a Leica TCS-SP2 laser scanning confocal microscope equipped with appropriate lasers for simultaneous imaging of up to four fluorophores. Digital data will be archived to compact disk or DVD and prepared for publication using Adobe Photoshop software (Adobe Systems Inc.; MountainView, Calif.).

Live Cell Brightfield Video Microscopy: Cells are plated on a glass coverslip coated with either 0.1% gelatin or 1 ng/ml laminin and polyornithine. Coverslips are mounted in a closed perfusion chamber (Warner Instruments, Holliston, Mass.) and attached to a syringe containing CO₂ equilibrated media. Media was perfused daily for up to 4 days. A Nikon inverted microscope (TE300) equipped with Hoffman Modulation Contrast and fluorescence, is temperature controlled with a Nikon environmental chamber. Cells are imaged with a low light camera (Hamamatsu, USA) and time lapse recording controlled by Metamorph (Universal Imaging), which permits interleaved acquisition of Brightfield and multiple fluorescence wavelengths.

Time Lapse Confocal Microscopy: Live cells were imaged by real-time-spinning disk confocal microscopy (Perkin Elmer Ultraview LCI equipped with a Krypton-Argon ion laser) to minimize photobleaching and to discriminate nuclei movement in multilayered ES cell colonies. GFP-H2B transfected cell colonies in Matek glass bottomed 35 mm dishes were mounted in a chamber (Warner Instruments) perfused with humidified CO₂. The Nikon TE2000E inverted microscope was enclosed in a LIS Systems temperature control chamber to eliminate thermal fluctuations and maintain focus for long-term experiments. Cells were imaged with 20× NA 0.45 plan fluor objectives, or 40× and 60× planapo 1.4 NA objectives. Under these conditions, colonies of GFP-H2B transfected ES cells were maintained for up to 7 days of continuous observations. Light levels were reduced until time-lapse sequences of colonies (50 slices, 24 hr duration, 10,000-70,000 images) could be acquired without abeyance of mitosis or increase in cell death. DNA was labeled with 1 μM Syto-16 (Molecular Probes, Carlsbad Calif.) diluted in culture media. Cells were observed in the presence of Syto-16 for up to 3 hrs without photodamage. Image stacks are further processed with Volocity (Improvision) to produce 3-D reconstructions, multispectral movies of XY, XZ, and YZ cross-sections: Live GFP-H2B labeled stem cells have been observed for >11 days continuously. More than 20,000 images (60 GB) in 4 dimensions can be acquired with no harmful effect on cell movement or mitotic progression.

FRAP and FLIP Photobleaching. GFP-H2B transfected cells were pattern photobleached with 488 argon ion laser light using a Leica TCS SP2 scanning laser confocal microscope using the 63× 1.4NA planapo lens with the aperture set to 3 um (2× airy disk). Cells were imaged with low intensity light (4-8% of bleaching intensity) for 10 images, photobleached during 30-60 frames, for 40-90 s and then imaged for and additional 20-100 frames at low intensity. Intensity profiles were calculated from submaximal rectangular areas selected to avoid edge effects and movement artifacts. Fluorescence photobleaching will be used to provide fiducial marks for studies of chromatin dynamics and subunit exchange. Photobleaching artifacts are possible due to stray light and photodamage. Photobleaching artifacts were evaluated by measuring photobleaching outside of the bleached zone in live cells and will extend these controls to cells fixed in 1.6% paraformaldehyde (60 min) to provide “immobile” proteins. Fixed cells will also be used to determine background and autofluorescence levels and to measure bleaching in adjacent zones due to scattered light. Additional controls for the unlikely possibility of reversible GFP photobleaching during FRAP include varying the size and intensity of the bleached zone. For example bleaching the entire nucleus should result in no short-term recovery of fluorescence. Nuclear rotation in the XY plane is common in stem cell colonies. These cells will be excluded from recovery analysis. Bleached zones will be analyzed by two methods. First beached zones provide a histone marker to aid in studies of speed and direction of motion by conventional methods (using Metamorph kymographs and related analysis, Molecular Devices Corp., Sunnyvale, Calif.). Within bleached zones, FRAP also occurs by exchange of GFP-histone as well as large scale mixing. GFP-histone association/dissociation kinetics will be analyzed assuming stochastic movement or exchange. If sampling time is fast enough, exchange between two populations of histone-GFP recovery should occur exponentially and be governed by R=C+P(1−exp^{kt}), where R=relative intensity, C=constant value at time zero, P=plateau value, k=association constant, and t=time. The half time for recovery can be calculated from k: t_{1/2}=ln(½)/k. In addition to the software provided by Leica, curves will be analyzed with GraphPad Prism (GraphPad Software, Inc., San Diego, Calif.) and differences plotted with Instat (GraphPad). If there are two exchanging populations (“rapid” and “slow”), recovery is governed by R=C+P(1−exp^{kt})+P2(1−exp^{k2t}) where P1 and kl refer to population 1 and P2 and k2 to population 2.

FLIP: The GFP-H2B FLIP and FRAP experiments are expected to agree on the presence or absence of a diffusible histone component. Quantitative evaluation of these experiments will include the dependence on Oct-4 expression, detected by immunocytochemistry after completion of the experiment. FLIP may be an artifact of overexpression and so the dependence on total GFP expression in the cells will be evaluated. GFP-H2B and GFP-H3 will be extracted with 0.5% Tx-100 and analyzed for % FLIP and the dependence of this loss on initial intensity and on Oct-4 expression. Further, quantitative analysis of diffusion coefficients is not anticipated to be in the scope of these experiments. However, if required, the diffusion coefficients could be calculated by classical means from a bleached spot with Gaussian profile, rather than a bleached pattern, assuming that recovery over a time span of a few seconds is due to diffusion, not movement. Analysis, if required would be carried out as in Phair and Misteli (Nature. 2000 404(6778):604-9).

Dynamic imaging of cell tracer-labeled chimeric embryos_by a spinning microlens array confocal microscopy (UltraView; Perkin-Elmer, Boston, Mass.) will be performed as follows: Successful reaggregations will be transferred to glass bottom 35 mm dishes (MatTek, Ashland, Mass.), mounted on a warming plate (Warner Inst., Holliston, Mass.), and the CO₂ regulated with a home built acrylic chamber and humidifier. Oil immersion objectives are warmed with a temperature controller (Bioptechs, Butler, Pa.): An automated Nikon 2000E inverted microscope is integrated with the Perkins Elmer Ultraview LCI spinning disk confocal microscope to record time-lapse image stacks at 488, 514 and 647 nm excitation. Image stacks are further processed with Volocity (Improvision) to produce 3-D reconstructions and multispectral movies of XY, XZ, and YZ cross-sections. Chimera constructs will be recorded beginning 24 hr after reaggregation and continue each 24 hr until development ceases or attains expanded blastocyst. At the end of development, chimeric embryos will be fixed in 2% formaldehyde overnight, which preserves the GFP staining profile, and then counterstained with 1 μM Toto-3 (Molecular Probes, Eugene, Oreg.) to label the DNA. A mouse GFP antibody (1:50; Molecular Probes) may also be employed to immunostain fixed embryos to confirm GFP protein expression in selected embryos. Alexa-568 antimouse IgG secondary antibody is applied to detect GFP blastomeres in these fixed samples. Trophectoderm and inner cell mass cells will be stained according to published protocols. All static samples will be optically sectioned using a Leica TCS-SP2 laser scanning confocal microscope.

Quantitative image analysis: Confocal image stacks were acquired with the Leica TCS SP2 or the Perkin Elmer Ultraview LCI confocal microscope under standardized conditions. Pseudocolor representation of time sequences can be prepared in Photoshop. Quantitative image analysis will be performed with a combination of Volocity, a 3D software for voxel quantification (size, intensity and distribution) and 3-D display. More sophisticated image quantitation of shape parameters is provided by Metamorph. Image slices were selected, background subtracted and further processed with Metamorph (Universal Imaging). Nuclear masks were prepared from DNA images derived from Toto-3 staining. The nuclear image processed by flat fielding, applying a median filter and morphological opening to remove small specks followed by morphological closing to eliminate small holes. A 1-bit mask was prepared by thresholding the nuclear image, which was then used to select nuclei from the oct-4 and lamin images by image multiplication and normalization. Each nuclear object in the field was identified and analyzed by the Image Morphology Tool to tabulate the size, circularity, and intensity of nuclei. Individual nuclei and their associated data were sorted manually for quality and degree of differentiation. Statistical differences will be determined with Student T tests. In addition, novel texture analysis tools have been developed using MatLab (Jeffreys et al., submitted) for quantitatively evaluating stem cell colony morphology texture and nuclear dynamics of fluorescent markers of pluripotency (Sammak et. al, in review). The same texture analysis applied to nuclei will provide very powerful, quantitative analysis for distinguishing the increased granularity of histone distributions. This analysis will augment the current proposal without additional expense.

Negative stain electron microscopy: Pellets of pluripotent HSF-6 cells are fixed in 2.5% Glutaraldehyde overnight. Cells are then given three 15 minute washes in PBS, and then incubated in 1% OsO₄ (Osmium) with Potassium Ferricyanide for an hour at 4° C. After that the sample is once again given three 15 minute washes in PBS, and then dehydrated in 30% EtOH (15 min), then 50% EtOH (15 min), then 70% EtOH (15 min), then 90% EtOH (15 min), then three times in 100% EtOH (15 min each). The sample is then given two 10 minute washes in Propylene Oxide (10 min each), followed by an hour incubation in 1:1 Epon/P.Oxide mixture. It is then incubated overnight at 4° C. in 100% Epon, followed by three separate, hour-long incubations in 100% Epon. The pellet is then embedded and cured at 37° C. for 24 hours, followed by a 48-hour incubation at 60° C. The sample is sectioned at 65 nm and mounted on 200 mesh copper grids and then heavy metal counter stained with Uranyl acetate and lead citrate.

Immunoelectron microscopy: Pellets are fixed with a 2% paraformaldehyde and 0.01% glutaraldehyde solution for one hour. Gelatin at 3% is then added to the pellets to harden them. The pellet with the gelatin is fixed once more for 15 min. The samples are placed in PVP and refrigerated for 24 hours. After this, they are frozen and sectioned (65 nm sections). Once sectioned, they are labeled by immunocytochemistry as necessary.

### Part V

### Primate Techniques

### EXAMPLE V

### 1. Providing Nonhuman Primate Gametes and Embryos

Primate acquisition and viral/pathogen status: All nonhuman primates utilized in these experiments will be purchased from USDA licensed nonhuman primate dealers or directly from nonhuman primate research facilities (e.g., National Primate Research Centers, pharmaceutical companies, universities, etc.). Cercopithecine herpes virus 1 antibody negative animals will be purchased whenever possible, although healthy antibody positive animals may also be purchased. To avoid accidental cross-contamination, antibody negative and antibody positive animals will be housed separately. Simian Retrovirus Type D (SRV) animals will not be purchased due to the potential for this viral infection to invalidate studies involving macaques (Schroder, 2000, Contemporary Topics in Laboratory Animal Science 39:16-23). Furthermore, no SIV or STLV positive animals will be purchased for this colony.

Determining mtDNA polymorphisms in rhesus colony: is accomplished by analyzing the D-loop of the rhesus monkey mtDNA genome through nested PCR and automated sequencing. Briefly, the hypervariable regions of the D-loop of the mitochondrial genome will be analyzed from blood and/or tissue samples. These products will then be sequenced using direct sequencing methods for mtDNA according to Hopgood et al. (Hopgood, 1992, Biotechniques 13:82-92). Unique polymorphisms allow specific primers for PCR amplification to be designed, a technique known as allele specific-PCR (AS-PCR). Alleles specific to certain animals will be identified by AS-PCR and resolved on 4% agarose gels. mtDNA products from AS-PCR will be confirmed by restriction fragment length polymorphic (RFLP)-PCR analysis as in St. John et al. (St John et al., 2000, J Androl 21:189-199) and Southern Blotting (Larsson et al., 1998, Nat Genet 18:231-236). Chimeras made in Part I will use embryos from animals in Part V with distinct polymorphisms.

Follicle stimulation regimen: Ovarian stimulation of female rhesus monkeys exhibiting regular menstrual cycles is induced with exogenous gonadotrophins (VandeVoort, 1989, J In Vitro Fertil Embryo Transfer 6:85-91; Zelinski-Wooten, 1995, Hum Reprod 51:433-440). Beginning at menses, females are down-regulated by daily subcutaneous injections of a GnRH antagonist (Antide; Ares Serono, Aubonne, Switzerland; 0.5 mg/kg body weight) for 6 days during which recombinant human FSH (r-hFSH; Ares Serono or Organon Inc., West Orange, N.J.; 30 IU, i.m.) is administered twice daily. This is followed by 1, 2 or 3 days of r-hFSH+r-hLH (r-hLH; Ares Serono; 30 IU each, i.m., twice daily). Ultrasonography is performed on day 7 of the stimulation to confirm adequate follicular response. When there are several follicles 3-4 mm in diameter, an i.m. Injection of 1000 IU r-hCG (Serono, Randolph, Mass.) is administered for the induction of ovulation.

Follicular aspiration by laparoscopy: Follicular aspiration is performed approximately 27 h post-hCG via laparoscopy. Stimulated females are anesthetized with an intramuscular dose of ketamine (10 mg/kg), intubated with a cuffed endotracheal tube, fitted with a 22 gauge angiocath in the radial or saphenous vein, sterilely prepped for surgery, and maintained on isoflurane anesthesia to prepare for them for laparoscopy. Oocytes are aspirated from follicles using a needle suction device lined with Teflon tubing. Briefly, a 10 mm trocar is placed through the abdominal wall and a laparoscope is introduced. The ovaries are visualized via a camera attached to the inserted laparoscope. Two small skin incisions facilitate the insertion of 5 mm trocars bilaterally. Grasping forceps are introduced through each trocar to fixate the ovary at two points. Once stabilized, a 20-gauge stainless steel hypodermic needle with teflon tubing is attached to a vacuum regulator. The tubing is first flushed with sterile TALP-Hepes, supplemented with 5 IU/ml heparin and then inserted through the abdominal wall and into each ovary. Multiple individual follicles are aspirated with continuous vacuum at approximately 40-60 mm Hg pressure into blood collection tubes containing 1 ml of TALP-Hepes/heparin medium maintained at 37° C. Collection tubes are immediately transported to a dedicated primate oocyte/zygote laboratory for oocyte recovery and evaluation of the maturation stage.

Collection and evaluation of rhesus oocytes: The contents of each collection tube are diluted in TALP-Hepes supplemented with 2 mg/ml hyaluronidase to facilitate removal of the cumulus cells (Hewitson et al., 1998, Hum Repro 13:3449-3455). Oocytes are rinsed and then transferred to pm-equilibrated TALP medium containing 3 mg/ml BSA (TALP). Metaphase II-arrested oocytes, exhibiting expanded cumulus cells, a distinct perivitelline space, and first polar body, are maintained in TALP for up to 8 h before fertilization. Immature oocytes are matured in TALP plus hormones for up to 24 h (Bavister, 1983, Biol Reprod. 28:983-999; Boatman, 1987, “In Vitro Growth of Non-Human Primate Pre- and Peri-implantation Embryos,” Plenum Press; Morgan, 1991, Biol Reprod 45:89-93).

Collection and preparation of rhesus sperm: Rhesus males of proven fertility have been trained to routinely produce semen samples by penile band electroejaculation using previously published techniques(Bavister, 1983, id.; Boatman, 1987, id.). These animals will be used to provide sperm for Part V, although no males will maintained. After liquefaction of the coagulated ejaculate, the liquid semen is removed and washed three times in 10 ml of TALP-Hepes by centrifugation at 400× G for 5 min. After resuspension of the pellet in 1 ml TALP-Hepes, a small sample is removed for morphological analysis. The remainder is counted, diluted to a concentration of 20×10⁶ sperm/ml in TALP in a 5-10 ml snap-cap tubes and incubated until use. For in vitro fertilization (IVF), sperm suspensions are incubated at 37° C. under 5% CO₂ in air for 6 h at which point 1 mM caffeine and 1 mM dibutyryl cyclic adenosine monophosphate (dbcAMP), are added for the final hour of incubation to stimulate hyperactivation (Bavister, 1983, id.). For intracytoplasmic sperm injection (ICSI), a 1 μl aliquot of sperm is removed and transferred to the manipulation dish.

Fertilization of rhesus oocytes by in vitro fertilization (IVF) and intracytoplasmic sperm injection (ICSI): For IVF, mature rhesus oocytes are fertilized in vitro using caffeine- and dbcAMP-stimulated, ejaculated rhesus sperm at a final concentration of 1.8×10⁵ sperm per ml in 100 μl TALP droplets under mineral oil. For ICSI, holding pipettes (O.D. 100 μm; I.D. 20 μm) and microinjection needles (O.D. 6-7 μm and LD. 4-5 μm), with a 50° bevel and a short, sharp, point (Humagen, Inc., Charlottesville, Va.) are mounted on a Nikon TE300 inverted fluorescent microscope equipped with Hoffman modulation contrast (HMC) optics. The manipulation pipettes are controlled by hydraulic Narishigi manipulators attached to a Hamilton syringes. Injections are carried out at 35° C. in 100 μl drops of TALP-Hepes placed in the lid of 100 mm tissue culture dish, covered with light mineral oil (Hewitson et al., 1998, id.). Capacitated, hyperactivated sperm are diluted 1:10 in 10% polyvinylpyrrolidone (PVP; Sigma). A single sperm is aspirated tail-first from the sperm-PVP drop into the microinjection needle and transferred to the oocyte-containing drop. Oocytes are immobilized with the polar body at 11 o'clock and the injection needle is inserted through the zona into the cytoplasm. The oolemma is breached by gentle cytoplasmic aspiration, which is then released with the sperm back into the oocyte. Microinjected oocytes are examined with a x40 HMC objective to verify the presence of a single sperm within the cytoplasm. ICSI can also be used as a method to produce androgenotes (see below). Fertilization is scored between 5-12 hrs post-insemination by confirming extrusion of the second polar body and by the presence of two pronuclei in the cytoplasm. Zygotes are cultured in fresh CO₂-equilibrated TALP medium until the 2-cell stage. After completion of the first cleavage division (24-28 h post injection), 2-cell embryos are co-cultured in CMRL+10% FCS (Hyclone Laboratories, Inc., Logan, Utah) on Buffalo rat liver cell monolayers (BRL 1442; ATCC, Rockville, Md.) seeded in 100 μl drops overlaid with oil. Two-cell embryos will be used in Part I and Part III to produce tetraploid embryos; and 8-cell embryos will be provided for chimera formation. Blastocysts will be used to isolate inner cell mass cells for ES cell derivation.

Embryo Sexing. Simultaneous FISH will be performed for several known chromosome sequences in order to determine embryo sex. Blastomere biopsy from 8-cell embryos (˜60 hours post ICSI) are based on the methods of Handyside et al. (Handyside et al., 1990, Nature. 344(6268):768-70). Each embryo is immobilized by suction with a flame-polished holding pipette held in one micromanipulator. The second micromanipulator with a double holder controls a drilling pipette (internal diameter 10 μm) containing acid Tyrode's solution (pH 2.4) and a sampling pipette (internal diameter 30 pm) containing buffered medium. The drilling pipette is placed in close contact with the zona pellucida and a hole made with a controlled stream of acid Tyrode's solution. Immediately, the zona is penetrated and this pipette removed, then the sampling pipette is pushed through the hole. One blastomere is then removed by gentle suction. In all cases, an interphase nucleus is observed in the isolated blastomere. For FISH, the single blastomere is transferred to 0.1% sodium citrate at 30° C. for 20 min. After a brief immersion in cold methanol/acetic acid (3:1 ratio), the fixed cells are transferred to a clean slide and dried on a warming plate (Clyde, 2001; Vollmer, 2000). Fixed blastomeres are then sexed using a premixed cocktail of centromeric probes for chromosomes 18, X and Y (MultiVysions; Vysis) according to the techniques of Clyde et al. (Clyde et al., 2001). Slides are treated for 30 min at 37° C. in RNAase and placed into 0.01 M HCL containing 0.005% Pepsin Slides for 5 min. After rinsing in phosphate-buffered saline (PBS; pH 7.4), the slides are treated with 1% formaldehyde in PBS for 2 min at 20° C. and then blocked in 0.5% nonfat milk powder in PBS for 1 hr at 37° C. Slides are probed with a mixture of 52 different chromosome paints (SpectraVysion; Vysis, Downers Grove, Ill.) after denaturing at 74° C. for 5 min. Specimens are dehydrated and denatured further in 70% formamide/2×SSC (30 mM of C₆H₅Na₃O₇.2H₂O with 300 mM of NaCl, pH 7.0) for 5 min at 76° C. The M-FISH probe is applied overnight at 37° C. Posthybridization washes are performed in three washes of 50% formamide/2×SSC for 10 min each at 46° C. and a final rinse in 2×SSC/0.1% Triton X-100 detergent for 5 min at 46° C. Slides are air-dried in the dark and then mounted in antifade solution (Vectashield; Vector Laboratory, Inc.) containing diamidino-2-phenylindole (DAPI) at 40 ng/ml. The chromosome spreads are viewed by conventional epifluoresence microscopy using Smartcapture software (Vysis). Sexed 8-cell embryos will be used in Parts I and III for chimera formation.

### EXAMPLE V

### 2. Techniques Related to Pregnancies

Selection of recipients for embryo transfer. Rhesus females with normal menstrual cycles synchronous with the oocyte donor are screened as potential embryo recipients. Screening is performed by collecting daily blood samples beginning on day 8 of the menstrual cycle (day 1 is the first day of menses) and continuing until ovulation (typically between days 13-15). Blood samples are assayed serum progesterone and estrogen levels using automated ELISA kits from Biomeriux (Durham, N.C.), on a Biomeriux Vidas machine. When serum estrogen increases 2-4 times that of base levels, the estrogen surge has occurred and ovulation usually follows within 12 to 24 h. Timing of ovulation can be detected by a significant decrease in serum estrogen and an increase in serum progesterone to above 1 ng/ml.

Embryo transfer for cleavage stage embryos: Surgical embryo transfers are performed on day 2, 3 or 4 following ovulation by transferring two 8-cell to morula stage embryos into the oviduct of the recipient. All transfers are performed via laparoscopy whereby the oviduct is cannulated using a Cook embryo transfer catheter, preloaded with the embryos in TALP-Hepes medium. Embryos are expelled from the catheter in about 0.05 ml of medium while the catheter is withdrawn. The catheter is flushed with medium into a Petri dish following removal from the female to ensure that the embryos are successfully transferred. Control and manipulated cleavage stage embryos will be obtained from the various questions addressed in Part 1 for embryo transfer.

Natural Mating: is necessary for the establishment of control pregnancies, staged pregnancies for fetal gonad collections and the collection of in vivo derived blastocysts for imprinting studies. For pregnancy establishment, female rhesus monkeys are stimulated as described above and then bred with males in our natural breeding program. Briefly, on the first day of menses, the average cycle length is calculated for each female using the individual menses report from the PDC Vivo database. The female is then placed with a male two days prior to the calculated ovulation date and stays with that male for 5 days (two days before and two days after ovulation). The breeding pair is observed during that period and any mating behaviors recorded, as well as documenting evidence of mating (i.e. sperm plug in cage pan). Females are then returned to their home cage until ultrasound on Day 18 and Day 30. Success rates are typically at 33% per mating. In vivo derived morulae and early blastocysts are flushed from the uterus on days 5 and 6 of pregnancy, respectively, as previously described (Seshagiri and Hearn, 1993, Hum Reprod 8:279-287; Thomson et al., 1994, J Med Primatol 23:333-336; Wolfgang et al., 2001, J Med Primatol 30:148-155). Timed staged pregnancies are required for Parts I, II and IV. In vivo derived blastocysts are provided to Part I for DNA methylations studies.

Pregnancy confirmation: Pregnancy is visually confirmed by transabdominal ultrasound on day 16-30 post-transfer. During ultrasound, mean gestational sac size, yolk sac diameter, greatest length, and embryonic heart rate measurements are collected to approximate the gestational age of the conceptus. These are compared to similar measurements made from IVF and natural pregnancies (Tarantal, 1988, Am J Primat 15:309-323). Ultrasound is performed monthly, to determine developmental normalcy. A blood sample is also collected from all ET recipients between days 16-20 post ET and is sent to UC Davis for analysis of monkey chorionic gonadotropin levels (mCG); (Shimizu et al., 2001, Am J Primatol 54:57-62) to determine if an early pregnancy was possibly established, despite not being sustained.

Fetectomies for Epiblast and PG Cell Isolations: Fetuses will be harvested at between days 14-18 for epiblast and hypoblast isolations and days 22 and 30-day-old (prior to and after germ cell migration) for PG cell isolation using the following procedure. After a 12-hour fast, monkeys are sedated with ketamine (10 mg/kg IM) and taken to the surgical prep area. The animal's hair is clipped from the ventral abdomen, the abdomen is cleaned with betadyne solution, a cuffed endotracheal tube is placed in the trachea, and an angiocath is placed in a radial or saphenous vein to provide fluid therapy throughout the surgical procedure. Maintenance anesthesia is then provided by isoflurane gas (0.5-1.5%) vaporized in 100% oxygen delivered via the endotracheal tube, a sterile prep of the abdomen is performed using betadyne scrub, and the surgical field is draped using sterile drapes. Once an adequate plane of anesthesia has been reached, a midline abdominal skin incision is made from the umbilicus to the pubis, the linea alba is located and elevated, and the abdomen is entered using sharp dissection along the linea. The uterus is then exteriorized and surrounded with sterile laparotomy pads soaked in warm saline. A longitudinal incision is then made in the serosa along the body of the uterus.

This incision is gently deepened until the amniotic sac is barely visible. Mild blunt dissection is then utilized to tease the amniotic sac away from its uterine connections and the fetus is harvested within an intact amniotic sac. The uterus is then closed using absorbable suture in a Cushing over a Lembert pattern. The linea alba is then closed using 2-0 vicryl in a simple interrupted pattern. The SQ tissue is closed (if necessary) using 3-0 vicryl in a simple continuous pattern, and the skin is closed using 4-0 vicryl in a subcuticular pattern. Excised tissues will be used in Part VI for the derivation of epiblast and PG cells for use in Part II and Part III, respectively.

High risk pregnancy management: Ultrasound (U/S) imaging of macaque fetuses will be performed using a Sonoline Antares high definition system with 2D/grayscale, M-mode, color M-mode, and color Doppler capabilities. The average gestational age of a rhesus-macaque is 165 days. The gestational sac (GS) and crown-rump length (CRL) are evaluated within the first trimester, beginning at gestational day (GD) ˜14-18 days. The yolk sac, embryo proper, and developing heart rate can be visualized as early as -GD 21-25 which confirms pregnancy. These parameters, along with the assessment of the placenta and amniotic fluid amounts, will also predict early pregnancy loss (28%), abortions, and/or blighted ova that would otherwise go undetected. Starting ˜GD 31-33 the embryo displays isolated movements of the cranium, and begins to show flexion and extensions of the extremities. At the end of organogenesis ˜GD 46-47, whole body activity is observed. At ˜GD 50-60, the amnion and chorion fuse. Other body measurements recorded by U/S from GD 60 until birth include, i. biparietal diameter, ii. occipitofrontal diameter; iii. head area, iv. head circumference, v. abdominal area, vi. abdominal circumference, and vii. femur length (Tarantal, 1988, Am J Primat 15:309-323; Tarantal, 1990, J Med Primatol 19:47-58). Fetal sexing is accomplished during the second trimester, around GD 70-100, when there is minimal amniotic fluid and infant size to cause image obstructions. Pregnant females approaching delivery will be monitored using an Animal Video Server (AVS). The AVS is a digital multiplex recorder and broadcast server currently configured for approximately 7 days of digital video to be recorded. It currently supports 16 video camera signals and 5 remote vet staff can simultaneously connect to the video server. The high-resolution video cameras generate quality video pictures during daylight hours and overnight. This allows animals to remain undisturbed, but still be monitored, overnight when most pregnant females go into labor. When an animal appears to be going into labor, the appropriate staff is notified so that they can assist the delivery and/or survival of the neonate. The AVS can also be used to broadcast, record or archive any surgery or procedure, which can be used a vet-teaching tool.

Amniocentesis: Amniocentesis will be performed on pregnant females between days 55-70 of gestation. To prepare an animal for amniocentesis, ketamine is administered intramuscularly at a dose of 10 mg/kg. Once sedate, the animal is delivered to the veterinary procedure room and placed in a supine position on the examination table. The skin of the abdomen is then shaved with an electric trimmer and prepped thoroughly with betadyne scrub followed by an alcohol rinse. Once prepped, the abdomen is covered with sterile gel and imaged with an ultrasound probe covered with a sterile sleeve. Once the uterus is located within the abdomen it is scanned closely to determine the position of the fetus, umbilical cord and placenta. When a pocket of amniotic fluid is located, a sterile 20-22 gauge spinal needle is passed through the pregnant monkey's skin and uterus into the amniotic sac carefully avoiding contact with the fetus, umbilical cord and placenta. The stylet is removed from the spinal needle and a 10 cc syringe is then attached to the needle. Five to seven milliliters of amniotic fluid is aspirated from the sac and the needle is then removed slowly. Once the needle is removed, the amniotic sac is checked for any sign of hemorrhage. The aspirated amniotic fluid is placed in a sterile conical tube and transferred immediately to the laboratory for analysis. The pregnant female is monitored until fully recovered from anesthesia and the viability of the fetus is rechecked via ultrasound in one week. Fluid retrieved during amniocentesis is used in Part VI for cell isolation and culture. Cells are also used in Part 1 for fetal NT.

Caesarian section: Nonhuman primate dams who carry their fetuses to 10 days past their due date or who exhibit signs of placenta previa, placental abruption, or fetal distress will have their offspring delivered via caesarian section. After a 12-hour fast, monkeys are sedated with ketamine (10 mg/kg IM) and taken to the surgical prep area. The animal's hair is clipped from the ventral abdomen, the abdomen is cleaned with betadyne solution, a cuffed endotracheal tube is placed in the trachea, and an angiocath is placed in a radial or saphenous vein to provide fluid therapy throughout the surgical procedure. Maintenance anesthesia is then provided by isoflurane gas (0.5-1.5%) vaporized in 100% oxygen delivered via the endotracheal tube, a sterile prep of the abdomen is performed using betadyne scrub, and the surgical field is draped using sterile drapes. Once an adequate plane of anesthesia has been reached, a midline abdominal skin incision is made from the umbilicus to the pubis, the linea alba is located and elevated, and the abdomen is entered using sharp dissection along the linea. The uterus is then exteriorized and surrounded with sterile laparotomy pads soaked in warm saline. A longitudinal incision is then made along the serosa of the body of the uterus avoiding the placental discs. The tissue of the amniotic sac is identified and elevated using forceps and then fine surgical scissors are used to incise the sac. The fetus is grasped through the incision in the amniotic sac and the fetus is delivered. Blunt dissection is used to remove the placental discs and the uterus is then closed using absorbable suture in a Cushing over a Lembert pattern. The linea alba is then closed using 2-0 vicryl in a simple interrupted pattern. The SQ tissue is closed (if necessary) using 3-0 vicryl in a simple continuous pattern, and the skin is closed using 4-0 vicryl in a subcuticular pattern.

Any high-risk pregnancies will also be taken by C-section rather than risk losing the infants during labor and/or delivery. Once the fetus is delivered, the umbilical cord is clamped in two places approximately ¾ of an inch from the umbilicus and scissors are used to sever the cord between the two clamps. The infant is then transferred to a waiting team of assistants and resuscitation is initiated. Cord blood will also be collected at this time. While there has been some success in re-introducing the infant back to the dam after surgery, the dam often rejects the infant. All of the infants are therefore removed from their mothers at birth and raised in the PDC nursery. This overcomes confounding problems like differences in dam-rearing on the development of the infants. In addition, developmental and behavioral testing can not be performed on infants that are raised with their mothers as this causes undue stress on the animals and can also skew the results. Therefore all controls and experimental animals will be nursery-raised. Based on previous experiences, a sample size of 5 or more infants per group provides enough statistical power for analyses. In the event that fewer animals are produced per group, only descriptive data can be obtained.

Collection of cord blood and placentae: The umbilical cord is clamped immediately after caesarian section and cord blood and placental tissue collected for either mitochondrial DNA analysis or transgene expression as described in Part I. To facilitate cord blood collection after delivery of an infant, the umbilical vein is cannulated with a 22 gauge butterfly catheter prior to placental detachment as described by (Pafumi et al., 2002, Gynecol Obstet Invest 54:73-77). The catheter is fitted with an empty 10 ml syringe and blood is gently aspirated and then immediately transferred to blood tubes containing heparin. Multiple 5 mm×5 mm×5 mm cubes of placenta will also collected and analyzed immediately or snap frozen in liquid nitrogen and stored at −80° C. for future analysis. If pregnancies spontaneously abort, fetal and placental tissue will be obtained whenever possible for similar analyses.

Transplantation of nhpES cells into adult rhesus macaques: MHC haplotyping of primates used for transplantation studies will be performed. Based on rodent studies, nhpES cells will be transplanted subcutaneously, into the parenchyma of the testes and intramuscularly. If cells are rapidly rejected, cells may also be transfered under the capsule of the kidney, which is considered a more immunologically-privileged site and is easy to image by ultrasound and MRI. Prior to administration of subcutaneous, intramuscular, and intratesticular ES cells, the recipient animal will be anesthetized with an intramuscular dose of ketamine (10 mg/kg) and the hair over the administration site will be clipped and the skin will be prepared with betadyne scrub followed by an alcohol rinse to maintain sterility. Subcutaneous administration will be performed under the skin between the shoulder blades to reduce the chance of the animal traumatizing the administration area. Intramuscular administration will be performed in the large muscle groups such as the quadriceps or biceps by incising the skin located over the target muscle and injecting the ES cells superficially into the belly of the muscle. Post-intramuscular injection, the skin over the injection site is closed with 4-0 vicryl in a simple interrupted pattern. Testicular administration is performed by grasping one testicle firmly, moving it caudally and laterally away from the other testicle, puffing the skin of the scrotum tightly over the isolated testicle, and injecting the ES cells into the parenchyma of the organ. Renal subcapsular transplant of ES cells will be done via laparoscopy. Briefly, once the animal is insulated with CO₂ via a gas port inserted just cranial to the umbilicus, two small skin incisions facilitate the insertion of 5 mm trocars bilaterally. One kidney is stabilized using a fine curved forceps inserted through one trocar and a small incision is made in the renal capsule using scissors inserted through the other trocar. The capsulotomy is then closed using 4-0 vicryl in a simple interrupted pattern. The renal capsule is then gently lifted with the fine curved forceps and the ES cells are deposited beneath the capsule via polyethylene tubing inserted through one of the trocars. Each site listed above will be injected with 0.5 to 5×10⁶ ES cells. nhpES cells are derived by Parts VI and IV.

Monitoring teratoma progression and excision: The ES cell administration sites of the subcutaneous, intramuscular, and intratesticular inoculated animals will be monitored daily for evidence of inflammation, infection, or teratoma growth. Once per week, these animals will be anesthetized with an intramuscular dose of ketamine (10 mg/kg) and the ES cell administration site will be palpated for evidence of teratoma growth. Any growth will be measured with Vernier calipers and recorded in the animal's clinical record. Teratoma growth will also be evaluated monthly using MRI imaging as described in Part IV. Subcutaneous, intramuscular, and intratesticular teratomas will be excised when they grow to 1.0 cm in diameter. The administration sites will be monitored closely for evidence of inflammation and necrosis that is often associated with rejection. Prior to the excision of a teratoma, the animal will be sedated with an intramuscular dose of ketamine (10 mg/kg) and the affected area will be shaved of hair and prepped with betadyne scrub. The animal will then be intubated with a cuffed endotracheal tub, a radial or saphenous vein catheter will be placed, and general anesthesia will be induced and then maintained with 0.5-2.0% isoflurane gas. The skin over the subcutaneous, intramuscular, and testicular teratoma will be then be infiltrated with 1.0% lidocaine to alleviate post-surgical incisional pain. Subcutaneous teratomas will be excised by making an elliptical incision through the skin and subcutaneous tissue surrounding the abnormal growth and removing the affected area plus an additional ½-centimeter border of unaffected skin. The remaining subcutaneous tissue will be closed using 3-0 vicryl in a simple continuous pattern and the overlying skin will be closed using 4-0 vicryl in a subcuticular pattern. Muscle teratomas will be removed by incising the skin over the affected muscle, locating the teratoma within the affected muscle belly, removing the affected muscle tissue using sharp dissection, and closing the muscle belly using 2-0 prolene with interrupted horizontal mattress sutures. The subcutaneous tissue and skin over the muscle belly will be closed as described above. Testicular teratomas will be removed by performing a traditional, unilateral closed castration of the affected testicle. Post-teratoma removal, all animals will be treated with intramuscular oxymorphone (0.15 mg/kg) three times a day for three days to alleviate discomfort. Abdominal ultrasound and MRI imaging will be performed on a monthly basis to assess the presence and the progress of subcapsular renal teratomas. A mid-ventral laparotomy will be performed to remove renal teratomas that grow to greater than 1.0 cm in diameter.

### EXAMPLE V

### 3. Techniques to Study and Insure Health of Nonhuman Primate

Primate NICU and nursery: Infants born under all projects enter an intensive care nursery immediately after either caesarian section or vaginal delivery and are reared under established guidelines (Ruppenthal and Sackett, 1992, Research Protocol and Technician's Manual: A Guide to the Care, Feeding, and Evaluation of Infant Monkeys, Seattle: Infant Primate Reasearch Laboratory). The nursery is staffed 24 hours a day by a well-trained group of animal care technicians, veterinary technicians, and behaviorists. Infants are intensively monitored for various health (temperature, hydration, respiratory and nutritional status) and developmental parameters to reduce morbidity and mortality and to document progress for later analysis and comparison to other infants undergoing similar rearing experience. To ensure the best possible infant care, the PDC has outfitted the NICU and nursery with state-of-the-art medical equipment. The major pieces of equipment include an Ohmeda 7800 pediatric ventilator, a Phillips V24E modular patient monitor with ECG, respiratory, SPO2, expired CO2, and noninvasive blood pressure readouts and, and three infant incubators.

Infants remain in intensive care until they can maintain physiological homeostasis, are capable of self-feeding, and are otherwise healthy. As they mature, they are moved to a traditional animal holding room containing other nursery graduates but are continually monitored to assess developmental parameters. During this time, all infants are housed singly. This is done for several reasons: (i) infant development (social, emotional, cognitive, growth, etc.) is assessed on a scheduled basis under closely controlled conditions so that all receive identical stimulation and experience. This is important for both experimentally manipulated animals and control animals, and provides a comparison group for statistical analysis. Mother-rearing influences behavioral trajectories and behavior. Some mothers are overly protective and restrictive, others more relaxed and permissive. Some are more abusive than others. All influence behavioral development of offspring. Mothers may also reject their infants, necessitating nursery care of a subset of infants, confounding their histories; (ii) if mother-reared but then the infants are separated for assessments, the mothers react violently to separation, endangering infants. Also, repeated separations from their mothers negatively impacts emotional development of infants. These separations have been shown to have lasting impact on infant development; and (iii) it has been demonstrated that the separation from mothers early in life can be ameliorated through proper rearing experience provided the infants (daily, but not full-time peer contact, intensive stimulation provided by human handling, and providing contingent and non-contingent stimulation from learning, enriched housing, and cognitive assessments) for macaques (Sackett et al., 2002, Am J Primatol 56:165-183), and baboons (Crouthamel & Ruppenthal, 2001, Am J Primatol 54:86). These infants develop normal species-typical repertoires including being able to live in large social groups and developing normal dominance hierarchies, development of appropriate sexual behavior, parenting well. They have no differences in trauma, gastrointestinal illness, fetal loss, etc. (Sackett et al., 2002, id.). Furthermore, starting at 2 weeks of age, each infant receives 30 minutes a day of social play with one or more peers in large play cage. Their social development is also tested 3 times a week with other infants. This continues for up to a year, at which time, compatible animals are pair-caged.

Assessments of premature infants: Initial assessment of the preterm infant will begin immediately after a controlled delivery by evaluating key vital signs including respirations, pulse, and temperature. Particular attention will focus on respiratory status to evaluate for signs of respiratory distress (tachypnea, retractions, need for supplemental oxygen). Based on the severity of symptoms, infants may need a spectrum of care to include careful observation to supplemental oxygen to the need for mechanical ventilation and exogenous surfactant therapy. As part of the stabilization process, infants will require a regulated neutral thermal environment first provided by a mobile radiant warmer where ready access to the infant is available for stabilization of respiratory and circulatory systems. During this period, the insertion of peripheral intravenous lines and umbilical catheters for monitoring purposes can be accomplished, if necessary. Umbilical arterial lines may be necessary to constantly monitor arterial blood pressure, assess oxygenation and ventilation status through arterial blood gas measurements, and phlebotomy for laboratory assessment of blood glucose and electrolyte status. Umbilical venous catheters can be used to supply necessary fluids (dextrose water solution initially followed by dextrose and electrolytes), antibiotics and eventually parenteral nutrition.

After the infant is stable, transfer to an enclosed neutral thermal environment (isolette) will be feasible. Subsequent monitoring of the preterm infant will include the use of cranial ultrasonography to assess the neonatal brain for the presence and follow-up of intraventricular hemorrhage (IVH) and periventricular leukomalacia (PVL). NH is strictly a lesion of the neonatal brain with inverse proportional risk and severity with decreasing gestational age. The pathogenesis is complex and multifactorial, but generally results from the interaction of circulatory disturbances resulting in fluctuations in cerebral perfusion pressure and the preterm infants' ability to regulate cerebral blood flow. Severity is graded by the amount of blood filling the ventricular system of the brain. Very severe hemorrhages (Grade IV) include a component of periventricular white matter damage secondary to venous infarction. PVL is white matter injury in the brain that can be focal, diffuse, and both diffuse and focal leading to permanent injury of the developing white matter tracts and eventually cerebral palsy, cognitive and behavioral disability. PVL can occur prior to birth, although the majority is related to postnatal aberrations in cerebral circulation that result from being premature and the need for mechanical ventilation. Both lesions, NH and PVL can also coexist in the premature brain. Cranial ultrasound can determine the extent and severity of NH as well as follow its course and assess for the development of post-hemorrhagic hydrocephalus. Similarly, PVL and the probable ultrasonographic precursor periventricular echogenicity (PVE), can be detected and followed over time. In the preterm human neonate, serial cranial ultrasound assessments are generally performed at day 7 of life and sometimes earlier based on the clinical scenario and then at days 14 and 30 to assess for the presence of hydrocephalus and degree of white matter injury. Magnetic resonance imaging is also adjuncts that can be employed to delineate the true extent of brain injury in cooperation with the Core A. This data is critical to correlate the future motor, cognitive, and behavioral assessments done on these infants. After the initial stabilization period, respiratory support will be weaned and parenteral nutrition started if appropriate all under the supervision of veterinary and neonatal support team.

Evaluation of behavior in infants: Stabilized infants are assessed by a series of protocols (Ruppenthal and Sackett, 1992, id.) to determine developmental normalcy including physical, emotional, cognitive, immunological, and behavioral assessments that have been employed for several years and have proven to be of worth in support of research utilizing the species used by the PDC. These protocols include measures such as: modified APGAR ratings, physical exams, weight gain/loss, reflex development, sleep/wakefulness and activity cycles, vital signs, food intake, development of object permanence, recognition memory, social behavior development, learning, anthropometrical growth and development, emotion, immune system development, and reactivity to mild challenges on a scheduled basis with some age-based and others daily, weekly, or monthly. With these measures it is possible to document normal development and identify deviations from norms through statistical analysis that may be due to factors such as experimental manipulations or may be naturally occurring. Intakes are recorded each feeding and daily weights are taken to assess weight gain per calorie consumed. Vital signs (heart rate, respiration rate and temperature) are recorded every & hours. Activity cycles (sleep, degrees of wakefulness, temperament) are recorded on a scheduled basis 24/7. In addition, infants are assessed for reactivity, clasping/grasping, righting, sucking, orientation and tracking of visual and auditory stimuli, and development of facial expressions three times per week to study development of reflexes. Progress through nursery is recorded for all animals (training to self-feed, ability to maintain homeostasis, ability to assimilate formula, health characteristics). At that time they emerge and are housed in a room containing other nursery graduates.

Socialization/development of infants: After emergence, infants are socialized daily in peer groups but housed in single cages. Continuous pair or group housing has been found to negatively impact development of normal social development (Chamove, 1973, Anim Behav 21:316-325; Ruppenthal, 1991, Am J Primatol 25:103-113). Their behavior is recorded utilizing a laptop computer and an exhaustive and exclusive scoring system (Sackett, 1973, Behav Res Methods Instrum Comput 5:344-348). In addition a check sheet is also employed to record the use of various apparatus, body postures, and communicative expressions. A series of protocols to assess the development of emotion, maturation, and cognitive development originally used in child development research but adapted for nonhuman primates are utilized. These include: Visual Recognition Memory, Cross Modal Recognition, Object Concept Assessment, Tests of Learning and Memory, and Black-White Discrimination and Reversal, as well as tests of temperament and immune function.

Evaluations of juveniles: Beginning at 12 months of age, animals will be group housed 24/7 in mixed sex groups of 4-6 for the ability to exist in a complex living situation and to allow for intensive interactions in social situations to be evaluated. Behavioral observations will continue to be recorded as well as: (i) Assessment of weight gain/loss; (ii) Onset of menarche in females and descent of testicles in males to document possible precocious puberty; and (iii) Learning. Learning tasks that have been shown not to be possible or extremely difficult in animals younger than one in most cases will be performed 5 days per week. Animals will be removed from their living groups daily to perform the assessments. These tasks include matching to sample: Animals view a sample object until they touch it, showing they are attending to the sample, followed by the sample and novel object are presented, with the familiar sample rewarded until the animal reaches criterion of 80% correct choices over at least 100 pair presentations. Following this the relationship is reversed with the unfamiliar sample being rewarded. This measures perseverance and emotionality’ as measured by performance and balking. Following the match-non match problem the animal will receive three-choice Oddity tasks, which consists of presenting three objects in a linear array, position randomized, with two objects the same and one different (which hides the food reward). This is a difficult problem for monkeys, and the error patterns and maximum performance might be indicative of performance by derivation.

Care of High Risk Infants: Infants born with physical handicaps will be reared in the nonhuman primate nursery under 24-hour care. If the physical abnormalities are manageable, the infant will be maintained in the vivarium and will be provided with appropriate clinical care fashioned to the animal's specific needs. If an infant's physical abnormalities are so severe that permanent intensive care is necessary, euthanasia will be elected. Euthanasia will be performed in accordance with the most recent recommendations of the AVMA Panel on Euthanasia.

As part of rearing protocols (Ruppenthal and Sackett, 1992, Id.) infants are socialized daily in peer groups to insure behavioral normalcy. Should individuals with physical or emotional handicaps affect normal social development steps have been developed and utilized to overcome such deficits. Infants reared in social isolation for the first several months of life exhibit a majority of avoidance behavior and extreme negative reaction when placed in a social situation. By grouping them with younger, less threatening animals who do not overwhelm these subjects, the isolate-reared subjects behaviors are transformed into positive, exploratory, playful and affiliative behavioral interactors. The same type of therapy has proven very useful when socializing infants with the chromosomal abnormalities trisomy 16 and 18 (Ruppenthal et al, 1983, Am J Mental Deficiency 87:81-92; Ruppenthal et al, 2004, Am J Mental Retardation 109(1):9-20). Nonsocial and non-interactive infants with these deficits have eventually exhibited positive, playful, explorative, affiliative behavioral profiles. Even infants born with visual deficits can be socialized as long as the play environment remains stable and unchanging. One study demonstrated that infants reared in total darkness were virtually impossible to distinguish from animals interacting in normal daylight, including developing normal “play-faces” as part of initiations to interact. These measures will be employed whenever needed.

### EXAMPLE V

### 4. Prepare, Support, and Transport Primates

Transport: On the day of imaging the monkey will be transported from her home cage via a standard nonhuman primate transfer box to a climate-controlled van equipped with a squeeze cage. For MRI, the animal will then be moved from the Pittsburgh Development Center (PDC) to the Mellon Institute that is approximately seven minutes away.

Anesthesia for MRI: Upon arrival at the Institute, the animal will be sedated with an intramuscular dose of ketamine (10 mg/kg) and atropine (0.04 mg/kg), removed from the squeeze cage, and taken to the NMR Center using the transfer box. Prior to the initiation of the imaging procedure, the animal will be intubated with a cuffed endotracheal tube and instrumented with a saphenous or radial vein catheter. As described previously by Benveniste et. al. (Benveniste et al., 2003, J Nucl Med 44:1522-1530), anesthesia will be maintained using intravenous propofol (Diprivan; AstraZeneca) at a dose of 120-300 μg/kg/min. During the imaging procedure, respiratory parameters will be maintained within acceptable limits by providing continuous mechanical ventilation. A pulse oximeter will be utilized to monitor oxygen saturation and pulse rate, body temperature will be maintained with a water circulating heating pad and a warm air source blowing directly into the bore of the MRI system, and a mixture of 0.9% NaCl and 5% dextrose will be administered intravenously at maintenance rates throughout the imaging procedure.

Post-imaging care: Post-imaging, the animal will be de-instrumented, recovered from anesthesia, placed back in the squeeze-cage of the van and transported back to its home cage at the PDC where it will be monitored closely for evidence of adverse reactions.

### Part VI

### Embryonic Stem Cell and Embryonic Germ Cell Techniques

### EXAMPLE VI

### 1. Isolation of Mouse Embrvonic Fibroblast Feeder Cells

Each supplier of human embryonic stem cells prefers a different strain of mice for derivation of feeder cells. For those cells obtained from WiCell, CF-1 mice will be used (Charles River, Wilmington Mass.). For cells from Bresagen, ICR mice will be used (Charles River) and for cells from ES Cell International, 129Sev mice will be used (Taconic, Germantown, N.Y.). Female mice at eight weeks of age are injected I.P. with 5 I.U. Pregnant Mare's Serum Gonadotrophin (PMSG). This injection is followed 48 hr later with 5 I.U. human chorionic gonadotrophin (hCG) followed by mating overnight. The next morning females are checked for vaginal plugs to confm coitus and separated from the males. Pregnant mice are sacrificed on day 12.5 of gestation by C02 asphyxiation. The uterine horns are dissected and the fetuses removed and placed into Dulbecco's phosphate buffered saline (PBS, Invitrogen, Carlsbad, Calif.): To help ensure a more pure population of feeders the heads and internal viscera are removed from the fetuses and the remaining tissues are rinsed in several washes of PBS. The tissue is transferred to a 100 mrn petri dish containing 2 ml of 0.05% TrypsinEDTA (Invitrogen, Carlsbad, Calif.). The

tissue is minced, an additional 5 ml of trypsin/EDTA added &d all 7 ml transferred to a 37” C incubator for 15 minutes. Two volumes of mouse embryonic feeder cell isolation media (CF-1 media, 90% DMEM, 10% fetal calf serum, 0. ImM MEM nonessential amino acids, 1.0% Penicillin/Streptomycin) are added and placed in a 50 ml centrifuge tube to settle after fust breaking up cells with gentle pipetting. After 15 minutes entire contents of tube are split evenly into tissue culture flasks.

When mouse embryonic feeder cells reach confluency they may be frozen in the following manner. Cells will be passaged using 0.05% Trypsin/EDTA and transferred to a 50 ml conical tube, counted and centrifuged at 800× g for eight min. After centrifugation cells are resuspended in freezing media. (30% CF-1 Media, 60% defined fetal bovine serum (HyClone, Logan Utah) and 10% DMSO (Sigma, St. Louis, Mo.)) and transferred to 1.5 ml cryovials (Nalgene, Rochester N.Y.). Cryovials are placed in a cell freezer (Fisher Scientific, Pittsburgh, Pa.) in a −80 C freezer overnight before being placed in liquid nitrogen for long-term storage.

Plating and Inactivation of Mouse Embryonic Fibroblast Feeder Cells: Mouse embryonic fibroblast cells (MEFs) are grown to confluence in tissue culture flasks and then mitotically inactivated by two-hour treatment with CF-1 media containing 10 μg/ml mitomycin C (Sigma, St. Louis, Mo.). Mitomycin C containing media is removed after two hours and the cells are washed three times with PBS before being harvested using 0.05% Trypsin/EDTA. Cells are transferred to a 50 ml conical tube, counted and centrifuged at 800× g for eight min. After centrifugation cells are resuspended in CF-1 media at a density of 7.5×104 cells/ml. These are transferred to culture dishes previously pretreated with 0.1% gelatin. Cells are plated at a density of −200K cells per well of a 6 well dish. Irradiation may be used as an alternative mechanism of inactivation. Cells will be irradiated with 3000 rad X-ray or gamma irradiation using a 2100 Cesium-source irradiator for seven minutes. Feeder plates prepared in either manner are used within two weeks of preparation.

Embryonic Stem Cell Culture and Passaging: All cell lines are obtained under executed and approved material transfer agreements (MTA's) negotiated between the National Institutes of Health approved distributors and the Principal Investigators. Each cell line will be cultured according to the distributors provided methodology as described below. The culture media for each line is:

H1, H7, and H9 (WiCell): Cells are cultured in 80% DMEM/F12 (Invitrogen, Carlsbad, Calif.), 20% Knockout Serum Replacement (Invitrogen), 1 mM L-glutamine (Invitrogen), 0.1 mM beta-mercaptoethanol (Sigma, St. Louis Mo.) 0.1 mM MEM non-essential amino acids (Invitrogen), and 4 ng/ml basic human recombinant FGF (Invitrogen).

HSF-6 and HSF-1 (UCSF): These cell lines are cultured and propagated in 80% DMEM high glucose (Invitrogen), 20% Knockout serum replacement, 1 mM L-glutamine, 0.1 mM MEM non-essential amino acids, 0.1 mM (3-mercaptoethanol and 4 ng/ml basic human recombinant FGF.

HES-3 and HES-4 (ES Cell International): These cells are cultured in 80% DMEM high glucose, 20% defined FBS (HyClone, Logan, Utah) 0.1 mM MEM non-essential Amino Acids, 0.5% Penicillin/Streptomycin, 2 mM L-Glutamine, 1% Insulin-Transferrin-Selenium supplement (Invitrogen), and 0.1 mM beta-mercaptoethanol.

Non-human Primate ES Cells (WiCell and PDC): These cells are cultured in 80% DMEM high glucose, 20% defined PBS, 0.1 mM MEM non-essential Amino Acids, 2 mM L-Glutamine, and 0.1 mM beta-mercaptoethanol.

The most consistent passaging technique for maintaining normal karyotype reportedly is manual passage as follows (Pera, 2004) A very fine glass needle is pulled over a flame or using a micropipette puller (Model P-87, Sutter Instruments, Novato Calif.). Individual colonies are sliced into small sections using the glass needle. One of two techniques will then be employed; either the sections displaying good compact morphology are detached using the glass needle followed by rinsing and pelleting as above or the differentiated sections of the colony can be scraped and rinsed away preceeding isolation of the good portions of the colony. This technique allows for the repeated passaging of embryonic stem cells with good morphology and is the recommended passaging technique of ES Cell International hESC lines (M. Pera, personal comm.).

Freezing and Thawing: In order to preserve early passages and to bank extra cells that are produced, samples of all embryonic stem cells will be periodically frozen in the following manner proven successful in our lab. Embryonic stem cells displaying an undifferentiated morphology (see above) are scraped as in passaging and transferred to a 15 ml conical tube. Each scraped well is then rinsed with 1 ml of the cell line specific media. This rinse is added to the 15 ml conical tube. The cells are pipetted gently to break up large clumps and pelleted at 200× g for 5 min. The supernatant is removed and 2 ml of the cell line specific media is added. The cells are gently pipetted and pelleted again as above. Supernatant is removed and 2 ml of freezing media (20% cell specific media, 60% defined fetal bovine serum, 20% DMSO) added. Cells are transferred to cryovials and placed in a cell freezer (Fisher, Pittsburgh, Pa.) in the −80° C. freezer overnight. The next day cryovials are transferred to liquid nitrogen for long-term storage.

Quality Control: To prevent loss of the undifferentiated phenotype strict quality control utilizing several assays (Immunocytochemistry 1.5.1, RT-PCR 1.5.2, karyotype analysis 1.5.3 and teratoma formation in NOD-SCID mice 1.5.4) is performed as described on a regular schedule as defined below.

Immunocytochemistry: As immunocytochemistry probes the heterogeneity of colonies it will be used whenever possible to determine the undifferentiated state of the human ESC's (M. Pera, personal communication). The hESC's will be probed for the positive ESC markers: Oct-4, SSEA-4, Tra 1-80 and Tra 1-61, as well as the negative ESC marker: SSEA-1. Immunocytochemistry will be preformed in one or more of the following manners. 1.) Culture dishes containing undifferentiated colonies will be fixed by addition of either 100% methanol 20° C. for 15 min followed by a 15 min wash in PBS+1% Triton X-100 (PBS−Tx, Sigma, St. Louis Mo.) for 15 min, or 2% paraformaldehyde in PBS for 40 minutes followed by quenching in PBS+150 mM glycine for 30 minutes subsequently washed out with PBS−Tx. After fixation, non-specific binding of the primary and secondary antibodies will be blocked when necessary by 20 minutes incubation in PBS with 3% non-fat dry milk. Primary antibodies are diluted in PBS+Tx and incubated on the coverslips for 40 min at 37° C. in a humidified chamber. After a 15 min wash in PBS+Tx a species appropriate fluorescently labeled secondary antibody is added to the coverslips for an additional 40 min. After another 15 min wash in PBS+Tx, 50M TOTO-3 (Molecular Probes, Eugene Oreg.) will be added for 20 min to label the nuclear DNA. Coverslips will be inverted onto slides and mounted in Vectashield anti-fade medium (Vector Labs, Burlinghame, N.H.) to prevent photobleaching (Navara et al., 2001).

Slides will be examined using both conventional immunofluorescence and laser scanning confocal microscopy. All imaging techniques are described in greater detail in Core A.

RT-PCR: Human and non-human primate embryonic stem cells are collected by scraping with pulled pipettes or microneedles as described above and pelleted by centrifugation at 200× g for 5 min. The cells are washed once with PBS and centrifuged again. RNA is isolated using an RNAqueous™-4PCR kit (Ambion, Austin Tex.) following manufacturer's instructions. Briefly, 100 μl of lysis/binding solution is added per 100-1000 cells and vortexed to lyse cells to homogeneity. An equal volume of 64% EtOH is added and gently mixed by inversion. This solution is added to the RNAqueous filter cartridge and centrifuged for one minute at 15,000× g. The filter is washed sequentially through buffers one and ⅔. After washing the filter is centrifuged for 30 sec to remove final traces of wash solution. The RNA is eluted from the filter by first placing in a fresh collection tube, adding 40 μl of preheated Elution solution (95° C.) and centrifuging at maximum speed for 30 seconds. DNase I buffer and 1 μl DNase I is added to the eluate and the mixture incubated for 30 min at 37° C. The DNase is inactivated and the RNA removed to a new tube.

To produce cDNA for PCR, 1 μg of total RNA is incubated for 10 minutes at 70° C. quick spun and placed on ice. The following reaction is prepared containing: 5 mM MgCl2, 1× RT buffer, 1 mM each dNTP, 1 unit/μl ribonuclease inhibitor, 15 unit/μg AMV RT, 0.5 μg random hexamers, and nuclease free water to a 20 μl final volume. This reaction is added to the RNA and incubated at room temperature for 10 min and then 42° C. for 15 min. Finally the sample is heated to 95° C. for 5 minutes, and immediately place at 4° C. for PCR or long-term storage at −20° C. Primers used for Oct-4 are forward cgaccatctgccgctttgag (SEQ ID NO: 33) and reverse ccccctgtcccccattccta (SEQ ID NO: 34). Primers for Nanog are forward accttccaatgtggagcaac (SEQ ID NO: 35) and reverse gaatttggctggaactgcat (SEQ ID NO: 36).

Cytogenetic Analysis to Assay Normal Karyotype: As a normal karyotype can be lost even when all pluripotency markers remain positive, a karyotype analysis will be performed on each resource line every 6 months.

A recent report suggests that chromosome arm 17q is frequently amplified in human embryonic stem cells (Draper et al., 2004, Nat Biotechnol 22:53-53). This interesting and important finding is preliminary in the small number of cell lines examined and that stem cell lines from only two labs were investigated. Thus as the stem cell core performs routine karyotyping, it be contributing to the body of knowledge regarding aneuploidy in embryonic stem cells. Current results have identified aneuploidy in cultured lines but to date there has been observed no overlap between cell lines or overlap with chromosome 17. It may be that individual lines are prone to different karyotypic anomalies or that laboratories select certain aneuploidies based on their laboratory practices (Pera, 2004, Nat Biotechnol 22:42-43). In either case a systematic documentation of aneuploidy will add to the existing knowledge of this field.

Teratoma Formation in SCID Mice: To assess the potential of the stem cells to contribute to all three-germ lineages, all stem cells will be assessed twice yearly for teratoma formation in SCID mice in the following manner. Human and non-human primate ESC's will be isolated by first scraping colonies with good morphology followed by a brief 5 min treatment with 0.25% Trypsin/EDTA to break up large fragments. Cells are pelleted at 800× G for 10 min and washed twice in PBS. Approximately 5×105−5×106 human embryonic stem cells (hES) or non-human primate embryonic stem cells (nhpES), are injected using a sterile 31 G needle into the testis of 8-12 week old NOD-SCID mice (Jackson Labs) using Institutional Animal Care and Use Committee approved protocols. Tumors will be grown until palpable or seven to eight weeks after injection. At the appropriate time mice will be euthanized by CO2 asphyxiation and tumors dissected, fixed in 4% formaldehyde, paraffin embedded, and examined histologically after hematoxylin and eosin staining. Formation of teratomas with cell lineages derived from ectoderm, mesoderm, and endoderm will be proof of the ES cell lines' pluripotency.

Data Collection and Record Keeping: Immunocytochemical and RT-PCR screening for the undifferentiated state will be performed bi-monthly, or in the case of cryopreserved lines, one passage before freezing and three passages after thawing. Karyotyping and teratoma formation will be performed every six months. All results will be recorded in a web-based database accessible to everyone in the whole Program Project for the purpose of long-term tracking. Results suggesting the loss of pluripotency will be repeated immediately and failure of two consecutive tests will result in the currently growing cell line being replaced from frozen stocks of known karyotype and pluripotency. Immediately after successful completion of pluripotency tests, cells will be frozen as described above to maintain a store of undifferentiated high quality cells.

Potential Difficulties and Limitations: Maintaining human and non-human primate embryonic stem cells in the undifferentiated state requires strict attention to the cultures and frequent assessment of pluripotency. Seemingly minor changes in culture conditions can result in the drastic and sudden loss of pluripotency. This will be guarded against by use of any advances in the stem cell field relating to the maintenance of pluripotency in cultured stem cells (e.g., Vallier et al., 2004, id.) and regarding the relationship between the stem cell environment and karyotypic stability (Draper et al., 2004, id.; Pera, 2004, id.).

### EXAMPLE VI

### 2. Establishing and Maintaining a Resource of Genetically and Epigenetically Characterized hESC, nhpESC, NTnhpESC and nhpEGC

hES, nhpES, NTnhpES and nhpEG cells will be maintained, their imprinting status monitored at a group of imprinted loci, and these cells will be available to each project.

Growth and maintenance of nhpES, NTnhpES and nhpEG cell lines. The nhpES, NTnhpES and nhpEG cells will be derived from specific matings as described in Parts I and III. Briefly, parents will be chosen based on analysis of their genomic DNA with primers specific for informative polymorphisms within imprinted genes. Matings will be established (see Part V for details) and embryos or fetuses harvested for derivation of nhpES, NTnhpES or nhpEG cell lines. Once established cell lines will be maintained using well-established protocols.

Analysis of imprint status in hES cell lines. Human and non-human primate embryonic stem cells are collected by scraping with pulled pipettes or microneedles as described above and pelleted by centrifugation at 200× g for 5 min. The cells are washed once with PBS and centrifuged again. RNA is isolated using an RNAqueous™-4PCR kit (Ambion, Austin Tex.) following manufacturer's instructions. Briefly, 100 μl of lysis/binding solution is added per 100-1000 cells and vortexed to lyse cells to homogeneity. An equal volume of 64% EtOH is added and gently mixed by inversion. This solution is added to the RNAqueous filter cartridge and centrifuged for one minute at 15,000× g. The filter is washed sequentially through buffers one and ⅔. After washing the filter is centrifuged for 30 sec to remove final traces of wash solution. The RNA is eluted from the filter by first placing in a fresh collection tube, adding 40μ1 of preheated Elution solution (95° C.) and centrifuging at maximum speed for 30 seconds. DNase I buffer and 1 μl DNase I is added to the eluate and the mixture incubated for 30 min at 37° C. The DNase is inactivated and the RNA removed to a new tube. To produce cDNA for PCR, 1 μg of total RNA is incubated for 10 minutes at 70° C. quick spun and placed on ice. The following reaction is prepared containing: 5 mM MgCl₂, 1× RT buffer, 1 mM each dNTP, 1 unit/μl ribonuclease inhibitor, 15 unit/μg AMV RT, 0.5 μg random hexamers, and nuclease free water to a 20 μl final volume. This reaction is added to the RNA and incubated at room temperature for 10. min and then 42° C. for 15 min. Finally the sample is heated to 95° C. for 5 minutes, and immediately place at 4° C. for PCR or long-term storage at −20° C. Informative polymorphisms were identified in four hES cell lines (H9, H7, hSF-6 and HES-3) for allele-specific expression of six imprinted genes [IGF2, IPW, KCNQ1OT1 (paternally expressed set); and H19, SLC22A18 and NESP55 (maternally expressed set)].

Analysis of imprint status in nhpES, NTnhpES and nhpEG cell lines. In Part III sets of primers to study imprinting status of maternally- or paternally-imprinted genes in nhps will be determined. Most of these primers will successfully amplify primate DNA (see Part III). RNA will be isolated from nhpES, NTnhpES and nhpEG cells essentially as described above. RNA will be converted into cDNA and amplified using primers spanning informative polymorphisms. The primers to be utilized are:

H19—Silenced on Paternal Chromosome

Zhang Y and Tycko B. Monoallelic expression of the human H19 gene Nature Genetics 1992 1:40-4

| | |
|---|---|
| Forward primer: | |
| cggacacaaaaccctctagcttggaaa | (SEQ ID NO: 37) |
| Backward primer: | |
| gcgtaatggaatgcttgaaggctgctc | (SEQ ID NO: 38) |

With human genomic DNA this creates a band of about 700 bp, and human cDNA about 620 bp. After the DNA band is digested overnight with RsaI, an informative pairing would be indicated by one uncut band (700 bp) plus one cut band (about 550 bp). Allele specific expression is detected by repeating the RsaI digest using the cDNA band from an informative source. Bi-allelic expression would be revealed by two bands—620 bp and 450 bp, whereas mono-allelic expression would show just one of the two bands.

IGF2—Silenced on Maternal Chromosome

Tadokoro K, Fujii H, Inoue T, Yamada M. Polymerase chain reaction (PCR) for detection of Apal polymorphism at the insulin-like growth factor II gene (IGF2). Nucleic Acids Research. 1991 19(24):6967

| | |
|---|---|
| Forward primer: | |
| cttggactttgagtcaaattgg | (SEQ ID NO: 39) |
| Backward primer: | |
| cctcctttggtcttactggg | (SEQ ID NO: 40) |

Same as above: DNA and cDNA create bands of about 300 bp. Digestion with ApaI cuts to 230 bp.

PEG1—Silenced on Maternal Chromosome

Pedersen I S, Dervan P A, Broderick D, Harrison M, Miller N, Delany E, O'Shea D, Costello P, McGoldrick A, Keating G, Tobin B, Gorey T, McCann A. Frequent loss of imprinting of PEG1/MEST in invasive breast cancer. Cancer Research 1999 59(21):5449-51

| | |
|---|---|
| Forward primer: | |
| tactaaaccagcatacccttac | (SEQ ID NO: 41) |
| Backward primer. | |
| gcagtcatcataaaggaatcag | (SEQ ID NO: 42) |

DNA and cDNA create bands of about 310 bp. Digestion with AflIII cuts to 260 bp.

PEG3—Silenced on Maternal Chromosome

Hiby S E, Lough M, Keverne E B, Surani M A, Loke Y W, King A. Paternal monoallelic expression of PEG3 in the human placenta. Hum Mol Genetics 2001 10(10):1093-100

| | |
|---|---|
| Forward primer. | |
| atgaatgcacagaaaccttcacttccag | (SEQ ID NO: 43) |
| Backward primer: | |
| ggtaagggtcaagtcctaggtgaaggtt | (SEQ ID NO: 44) |

Polymorphism 41 bp from end of ORF, in codon 1452. The frequent codon is cgc, which can change to cac if polymorphic. No restriction site therefore must sequence and observe the peak at the polymorphic site. The surrounding sequence is:

| | |
|---|---|
| cagctcttcaatgaccGcctgtccctcgcca | (SEQ ID NO: 45) |

SNRPN—silenced on maternal chromosome

Giacalone J, Francke U. Single nucleotide dimorphism in the transcribed region of the SNRPN gene at 15q12. Hum. Mol. Genetics 1994 3:379

| | |
|---|---|
| Forward primer: | |
| aaccaggctccatctactctttg | (SEQ ID NO: 46) |
| Backward primer: | |
| tcttgcaggatacatctcattcta | (SEQ ID NO: 47) |

DNA—about 1100 bp band, reduced to about 1000 bp when cut. cDNA—about 220 bp band, reduced to about 150 bp when cut. Use BstUI restriction enzyme.

IPW—Silenced on Maternal Chromosome

Wevrick, R., Kerns, J. A. & Francke, U. Hum. Mol. Genet. 3, 1877-1882 (1994).

| | |
|---|---|
| Forward primer: | |
| GGGAACTCTTCTGGGAGTGAATGTTATCA | (SEQ ID NO: 48) |
| Backward primer: | |
| GGGAGGTTCATTGCACAGAAATTTGG | (SEQ ID NO: 49) |

DNA- is a 1550 bp band. cDNA- is a 868 bp band. Polymorphism is C to T

KCNQ1OT1—Silenced on Maternal Chromosome

Lee, M. P. et al. Proc. Natl. Acad. Sci. U.S.A. 96, 5203-5208 (1999).

SNP1 in H9

| | |
|---|---|
| Forward primer: | |
| CAGCCACCTCTGTGGCGTGAATGTTCT | (SEQ ID NO: 50) |
| Backward primer: | |
| GCTCAAACCCGTCTCTGAAATGCACGG | (SEQ ID NO: 51) |

DNA and cDNA create bands of about 466 bp. Polymorphism is C to T

SNP2 in H7

| | |
|---|---|
| Forward primer: | |
| GATCCTCTCCAGGCAGCTTCTTCCACA | (SEQ ID NO: 52) |
| Backward primer: | |
| CATAAGGTAGGTAAGTTTGTGTCCCTG | (SEQ ID NO: 53) |

### ID NO: 53)

DNA and cDNA create bands of about 268 bp. Polymorphism is G to A

SLC22A18 DNA—Silenced on Paternal Chromosome

Onyango, P. et al. Proc. Natl. Acad. Sci. U.S.A. 99, 10599-10604 (2002).

| | |
|---|---|
| Forward primer: | |
| CTCTCACTGGGCAAGGCCACCT | (SEQ ID NO: 54) |
| Backward primer: | |
| GAGGAGGCTGCTCCACTCGCTG | (SEQ ID NO: 55) |

DNA- is a 315 bp band. Polymorphism is G to A

SLC22A18 cDNA

| | |
|---|---|
| Forward primer: | |
| GCCACTTCTCGGAGGAGGTGCT | (SEQ ID NO: 56) |
| Backward primer: | |
| GGAGCAGTGGTTGTACAGAGGCG | (SEQ ID NO: 57) |

cDNA- is a 231 bp band. Polymorphism is G to A

NESP55 DNA—Silenced on Paternal Chromosome

Hayward, B. E. et al. J. Clin. Invest. 107, 31-36 (2001).

| | |
|---|---|
| Forward primer: | |
| GGCTCCTTGTGCTGTCTGTCTTGTAG | (SEQ ID NO: 58) |
| Backward primer: | |
| CCACACAAGTCGGGGTGTAGCTTA | (SEQ ID NO: 59) |

DNA- is a 233 bp band. Polymorphism is T to C

NESP55 cDNA

| | |
|---|---|
| Forward primer: | |
| TCGGAATCTGACCACGAGCA | (SEQ ID NO: 60) |
| Backward primer: | |
| CACGAAGATGATGGCAGTCAC | (SEQ ID NO: 61) |

DNA- is a 1141 bp band. Polymorphism is T to C

PCR products will be sequenced. This analysis will be carried out on newly derived EG cell lines and on existing non-human primate ES cell lines for which parental information is known (see Part III). As data accumulates on improved hES or nhpES, NT-nhpES and nhpEG cell cultures, the effects of culture conditions on the stability of imprints will be examined. The sequence of the amplified fragments will be compared with that of the genomic sequences derived from the DNA of the parental samples to determine whether there is mono- or bi-allelic expression from that locus. This process will be repeated for as many genes as informative polymorphisms are found. As new informative polymorphisms are identified by the research carried out in Part III, the will be added to the reagents utilized.

Data Collection and Record Keeping: Immunocytochemical and RT-PCR screening for the undifferentiated state will be performed bi-monthly, or in the case of cryopreserved lines, one passage before freezing and three passages after thawing. Karyotyping and teratoma formation will be performed every six months. All results will be recorded in a web-based database for the purpose of long-term tracking. Results suggesting the loss of pluripotency will be repeated immediately and failure of two consecutive tests will result in the currently growing cell line being replaced from frozen stocks of known karyotype and pluripotency. Immediately after successful completion of pluripotency tests, cells will be frozen as described above to maintain a store of undifferentiated high quality cells.

Potential Difficulties and Limitations: Maintaining human and non-human primate embryonic stem cells and non-human primate embryonic germ cells in the undifferentiated state will likely require strict attention to the cultures and frequent assessment of pluripotency. Seemingly minor changes in culture conditions can result in the drastic and sudden loss of pluripotency. This will be guarded against by use of any advances in the stem cell field relating to the maintenance of pluripotency in cultured stem cells (e.g., Vallier et al., 2004, Stem Cells 22:2-11) and regarding the relationship between the stem cell environment and karyotypic stability (Draper et al., 2004, Nat Biotechnol 22:53-54; Pera, 2004, Nat Biotechnol 22:42-43). Currently there are over 200 animals within the PDC primate colony that can be screened in this way. Another approach will be to develop stem cell lines from inter-specific hybrids as has been carried out in the mouse. For example, Feinberg and colleagues developed EG cell lines from an interspecific cross of 129/SvEv and CAST/Ei mice (Onyango et al., 2002, Proc Natl Acad Sci USA 99:10599-10604). Since these mouse strains are evolutionarily diverged they carry many polymorphic loci that can be used for the purposes of analyzing both genetic mapping and genomic imprinting. This same approach could be used in non-human primates. Thus embryos can be generated from matings of Rhesus (Macaca mulatta) (164 days gestation) and Cynomolgus (Macaca fascicularis) (167 days gestation) monkeys. Genomic sequence analysis would be used to search for polymorphisms within the coding regions of imprinted genes. Once those polymorphisms had been identified, matings from informative animals would be established. Rhesus monkeys and other macaques can be successfully mated and produce offspring that are viable and fertile. Such crosses could be used to generate embryos from which stem cell lines could be derived. Analysis of genomic imprinting in F1 hybrids derived from different species would follow the same principal as described above.

The terms “a” and “an” and “the” and similar referents used in the context of describing the invention (especially in the context of the following claims) are to be construed to cover both the singular and the plural, unless otherwise indicated herein or clearly contradicted by context. Recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g. “such as”) provided herein is intended merely to better illuminate the invention and does not pose a limitation on the scope of the invention otherwise claimed. No language in the specification should be construed as indicating any non-claimed element essential to the practice of the invention.

Groupings of alternative elements or embodiments of the invention disclosed herein are not to be construed as limitations. Each group member may be referred to and claimed individually or in any combination with other members of the group or other elements found herein. It is anticipated that one or more members of a group may be included in, or deleted from, a group for reasons of convenience and/or patentability. When any such inclusion or deletion occurs, the specification is herein deemed to contain the group as modified thus fulfilling the written description of all Markush groups used in the appended claims.

Preferred embodiments of this invention are described herein, including the best mode known to the inventors for carrying out the invention. Of course, variations on those preferred embodiments will become apparent to those of ordinary skill in the art upon reading the foregoing description. The inventor expects skilled artisans to employ such variations as appropriate, and the inventors intend for the invention to be practiced otherwise than specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

Furthermore, numerous references have been made to patents and printed publications throughout this specification. Each of the above cited references and printed publications are herein individually incorporated by reference in their entirety.

In closing, it is to be understood that the embodiments of the invention disclosed herein are illustrative of the principles of the present invention. Other modifications that may be employed are within the scope of the invention. Thus, by way of example, but not of limitation, alternative configurations of the present invention may be utilized in accordance with the teachings herein. Accordingly, the present invention is not limited to that precisely as shown and described.

What is claimed is:

1. A composition comprising a chimeric primate embryo derived from non-human primate ES cells reaggregated with a fertilized primate embryo.

2. A method of generating a chimeric primate embryo comprising reaggregating nhp ES cells with biopsied fertilized primate embryos.

3. A method of determining the differentiation status of an embryonic cell comprising the steps of:
determining the cell transcriptional pattern of said embyronic cell;
comparing said embryonic cell transcriptional pattern to prototype transcriptional patterns, wherein each of said prototype transcriptional patterns are derived from an embryonic cell of a specific embryonic cell differentiation status;
determining which of said prototype transcriptional pattern most closely resembles said embryonic cell transcriptional pattern; and
assigning the specific embryonic cell differentiation status corresponding to said prototype transcriptional pattern most closely matching said embryonic cell transcriptional pattern, to said embryonic cell.

4. The method of claim 3, wherein the embryonic cell transcriptional pattern is determined by hybridizing labeled RNA isolated from single colonies of cells to microarray chips.

5. The method of claim 4, wherein the microarray chips display genomic DNA fragments originating from a species selected from the group consisting of mouse, human and Rhesus monkey.

6. The method of claim 5, wherein the microarray chips are selected from the group consisting of the Affymetrix® hg-u133+2 chip, Affymetrix® GeneChip® Rhesus Macaque Genome Array and Affymetrix® mg-u74Av2 chip.

7. The method of claim 3, wherein said specific embryonic cell differentiation status is selected from the group consisting of inner cell mass, epiblast, mesoderm, endoderm, ectoderm, lateral plate mesoderm, gut and neuroectoderm, extraembryonic mesoderm, amniotic ectoderm and visceral endoderm.

8. An isolated nhp PGC-derived pluripotential EG cell.

9. A method comprising the steps of:
mating non-human primates to establish a pregnancy;
terminating said pregnancy at between 28 and 45 days post coitus;
obtaining an nhp fetus;
isolating gonads from said fetus;
placing said gonads onto plates of feeder cells;
culturing said plates;
fixing and staining said plates for TNAP, a marker for PGCs;
detecting PGCs from said TNAP staining of said plates;
placing said PGCs into culture;
supplementing said culture with LIF and bFGF required for formation of EG cells, and the PGC mitogen forskolin;
feeding said culture daily and examining for said EG cells;
isolating said EG cells by picking;
plating said EG cells singly; and
testing said EG cells of resultant colonies for markers expressed on EG cells.

10. A differentiated cell derived from the EG cell of claim 8.

11. An embryoid body derived from the EG cell of claim 8.

12. A method comprising administering a composition comprising the differentiated cell of claim 10.

13. The method of claim 12, wherein said composition is administered to said patient to prevent, treat and/or alleviate the occurrence or negative effects of one or more diseases selected from the group consisting of: Alzheimer's, Parkinson's, muscular dystrophy, diabetes, stroke, and cardiovascular disease.

14. A method comprising the step of forming nhp EG cells by culturing nhp PGCs.

15. The method of claim 14 wherein isolation of said EG cells from said PGC culture is by picking.

16. The method of claim 15 further comprising the steps of plating said EG cells singly; and growing said plated single EG cells into colonies.

17. The method of claim 16 further comprising the step of testing said colonies for markers that are expressed on EG cells.

18. The method of claim 14 wherein said PGC culture is supplemented with LIF and bFGF and forskolin.

19. The method of claim 14 wherein said PGCs are derived from primate fetal gonads.

20. The method of claim 19 further comprising the steps of culturing said gonads on plates of feeder cells; and detecting said PGCs by fixing said plates, then staining said plates for the TNAP marker for PGCs.

21. The method of claim 19, wherein said gonads are derived from a fetus obtained at between 28 and 45 days post coitus.


SEQUENCE LISTING (61 sequences)

SEQ ID NO: 1 - DNA, 6 bp, Macaca mulatta
ggaccc

SEQ ID NO: 2 - DNA, 6 bp, Macaca mulatta
gggccc

SEQ ID NO: 3 - DNA, 6 bp, Macaca mulatta
gggccc

SEQ ID NO: 4 - DNA, 77 bp, Macaca mulatta
aagggccccagaaatcacaggtgggcacgtcgcgtctaccgccatctcccttctcacgggaattttcagggtaaact

SEQ ID NO: 5 - DNA, 261 bp, Macaca mulatta
  misc_feature (3)..(3): n is a, c, g, or t
ccnggacttgagtccctgaaccagcaaagagaaaagaagggccccagaaatcacaggtgggcatgtcgctgctaccgtcatctcccttctcatgggaattttcagggtaaactggccatcccaaaatagcaacaacccagactggctcctcactccttttccatcactaaaaatcacagagcagtcagagggacccagtactccanncaatatatnancatnncccttccttntnnanntaccttccaanntnantnaacc

SEQ ID NO: 6 - DNA, 300 bp, Macaca fascicularis
  misc_feature (219)..(219): n is a, c, g, or t
ccggacttgagttcctgaacagcaaagagaaaagaagggccccagaaatcacaggtgggcatgtcgctgctaccgtcatctcccttctcatgggaattttcagggtaaactggccatcccaaaatagcaacaacccagactggctcctcactcccttttccatcactaaaaatcacagagcagtcagagggacccagtaagaccaaaggaggacccctncntttcccttcnnnnnncttntntctctncactctnnnncnanttnatatntctccactccccttcctctannaannnnnn

SEQ ID NO: 7 - DNA, 300 bp, Macaca mulatta
  misc_feature (218)..(218): n is a, c, g, or t
ccggggacttgagtccctgaaccagcaaagagaaaagaagggccccagaaatcacaggtgggcatgtcgctgctaccgtcatctcccttctcatgggaattttcagggtaaactggccatcccaaaatagcaacaacccagactggctcctcactcccttttccatcactaaaaatcacagagcagtcagagggacccagtaagaccaaaggaggaanatnnnncnnnnncntntnnntntnncnttatntnnnnnanntnnnctttntnnnctnnnnnanncnanccnnnnnnannant

SEQ ID NO: 8 - DNA, 60 bp, Artificial sequence
  primer
attggatccaggccgctctggacaaaatatgaattctttttttttttttttttttttttt

SEQ ID NO: 9 - DNA, 27 bp, Artificial sequence
  primer
cggacacaaaaccctctagcttggaaa

SEQ ID NO: 10 - DNA, 27 bp, artificial sequence
  primer
gcgtaatggaatgcttgaaggctgctc

SEQ ID NO: 11 - DNA, 22 bp, artificial sequence
  primer
cttggactttgagtcaaattgg

SEQ ID NO: 12 - DNA, 20 bp, artificial sequence
  primer
cctcctttggtcttactggg

SEQ ID NO: 13 - DNA, 22 bp, artificial sequence
  primer
tactaaaccagcatacccttac

SEQ ID NO: 14 - DNA, 22 bp, artificial sequence
  primer
gcagtcatcataaaggaatcag

SEQ ID NO: 15 - DNA, 28 bp, artificial sequence
  primer
atgaatgcacagaaaccttcacttccag

SEQ ID NO: 16 - DNA, 28 bp, artificial sequence
  primer
ggtaagggtcaagtcctaggtgaaggtt

SEQ ID NO: 17 - DNA, 23 bp, artificial sequence
  primer
aaccaggctccatctactctttg

SEQ ID NO: 18 - DNA, 24 bp, artificial sequence
  primer
tcttgcaggatacatctcattcta

SEQ ID NO: 19 - DNA, 29 bp, artificial sequence
  primer
gggaactcttctgggagtgaatgttatca

SEQ ID NO: 20 - DNA, 26 bp, artificial sequence
  primer
gggaggttcattgcacagaaatttgg

SEQ ID NO: 21 - DNA, 27 bp, artificial sequence
  primer
cagccacctctgtggcgtgaatgttct

SEQ ID NO: 22 - DNA, 27 bp, artificial sequence
  primer
gctcaaacccgtctctgaaatgcacgg

SEQ ID NO: 23 - DNA, 27 bp, artificial sequence
  primer
gatcctctccaggcagcttcttccaca

SEQ ID NO: 24 - DNA, 27 bp, artificial sequence
  primer
cataaggtaggtaagtttgtgtccctg

SEQ ID NO: 25 - DNA, 22 bp, artificial sequence
  primer
ctctcactgggcaaggccacct

SEQ ID NO: 26 - DNA, 22 bp, artificial sequence
  primer
gaggaggctgctccactcgctg

SEQ ID NO: 27 - DNA, 22 bp, artificial sequence
  primer
gccacttctcggaggaggtgct

SEQ ID NO: 28 - DNA, 23 bp, artificial sequence
  primer
ggagcagtggttgtacagaggcg

SEQ ID NO: 29 - DNA, 26 bp, artificial sequence
  primer
ggctccttgtgctgtctgtcttgtag

SEQ ID NO: 30 - DNA, 24 bp, artificial sequence
  primer
ccacacaagtcggggtgtagctta

SEQ ID NO: 31 - DNA, 20 bp, artificial sequence
  primer
tcggaatctgaccacgagca

SEQ ID NO: 32 - DNA, 21 bp, artificial sequence
  primer
cacgaagatgatggcagtcac

SEQ ID NO: 33 - DNA, 20 bp, artificial sequence
  primer
cgaccatctgccgctttgag

SEQ ID NO: 34 - DNA, 20 bp, artificial sequence
  primer
ccccctgtcccccattccta

SEQ ID NO: 35 - DNA, 20 bp, artificial sequence
  primer
tcggaatctgaccacgagca

SEQ ID NO: 36 - DNA, 20 bp, artificial sequence
  primer
gaatttggctggaactgcat

SEQ ID NO: 37 - DNA, 27 bp, artificial sequence
  primer
cggacacaaaaccctctagcttggaaa

SEQ ID NO: 38 - DNA, 27 bp, artificial sequence
  primer
gcgtaatggaatgcttgaaggctgctc

SEQ ID NO: 39 - DNA, 22 bp, artificial sequence
  primer
cttggactttgagtcaaattgg

SEQ ID NO: 40 - DNA, 20 bp, artificial sequence
  primer
cctcctttggtcttactggg

SEQ ID NO: 41 - DNA, 22 bp, artificial sequence
  primer
tactaaaccagcatacccttac

SEQ ID NO: 42 - DNA, 22 bp, artificial sequence
  primer
gcagtcatcataaaggaatcag

SEQ ID NO: 43 - DNA, 28 bp, artificial sequence
  primer
atgaatgcacagaaaccttcacttccag

SEQ ID NO: 44 - DNA, 28 bp, artificial sequence
  primer
ggtaagggtcaagtcctaggtgaaggtt

SEQ ID NO: 45 - DNA, 31 bp, homo sapiens
cagctcttcaatgaccgcctgtccctcgcca

SEQ ID NO: 46 - DNA, 23 bp, artificial sequence
  primer
aaccaggctccatctactctttg

SEQ ID NO: 47 - DNA, 24 bp, artificial sequence
  primer
tcttgcaggatacatctcattcta

SEQ ID NO: 48 - DNA, 29 bp, artificial sequence
  primer
gggaactcttctgggagtgaatgttatca

SEQ ID NO: 49 - DNA, 26 bp, artificial sequence
  primer
gggaggttcattgcacagaaatttgg

SEQ ID NO: 50 - DNA, 27 bp, artificial sequence
  primer
cagccacctctgtggcgtgaatgttct

SEQ ID NO: 51 - DNA, 27 bp, artificial sequence
  primer
gctcaaacccgtctctgaaatgcacgg

SEQ ID NO: 52 - DNA, 27 bp, artificial sequence
  primer
gatcctctccaggcagcttcttccaca

SEQ ID NO: 53 - DNA, 27 bp, artificial sequence
  primer
cataaggtaggtaagtttgtgtccctg

SEQ ID NO: 54 - DNA, 22 bp, artificial sequence
  primer
ctctcactgggcaaggccacct

SEQ ID NO: 55 - DNA, 22 bp, artificial sequence
  primer
gaggaggctgctccactcgctg

SEQ ID NO: 56 - DNA, 22 bp, artificial sequence
  primer
gccacttctcggaggaggtgct

SEQ ID NO: 57 - DNA, 23 bp, artificial sequence
  primer
ggagcagtggttgtacagaggcg

SEQ ID NO: 58 - DNA, 26 bp, artificial sequence
  primer
ggctccttgtgctgtctgtcttgtag

SEQ ID NO: 59 - DNA, 24 bp, artificial sequence
  primer
ccacacaagtcggggtgtagctta

SEQ ID NO: 60 - DNA, 20 bp, artificial sequence
  primer
tcggaatctgaccacgagca

SEQ ID NO: 61 - DNA, 21 bp, artificial sequence
  primer
cacgaagatgatggcagtcac